(19) United States
(12) Patent Application Publication
(10) Pub. No.: US 2025/0186596 A1
(43) Pub. Date: Jun. 12, 2025

(54) A CONJUGATE CONSISTING OF OR COMPRISING AT LEAST A ß-GLUCAN OR A MANNAN

(71) Applicant: TRIDEM BIOSCIENCE GMBH & CO KG, Vienna (AT)

(72) Inventors: Markus MANDLER, Vienna (AT); Sabine SCHMIDHUBER, Vienna (AT); Achim SCHNEEBERGER, Vienna (AT); Carola WOLBER, Vienna (AT)

(21) Appl. No.: 18/842,272

(22) PCT Filed: Feb. 28, 2023
(86) PCT No.: PCT/EP2023/055022

§ 371 Date: Aug. 28, 2024

(30) Foreign Application Priority Data
Feb. 28, 2022 (EP) 22159194.4
Aug. 19, 2022 (EP) 22191217.3

(51) Int. Cl. A61K 47/61; A61K 47/64

(52) U.S. Cl. CPC A61K 47/61; A61K 47/64

(57) ABSTRACT

The invention relates to the use of β-glucans or mannans as C-type lectin (CLEC) polysaccharide adjuvants for B-cell or T-cell epitope polypeptides of alpha synuclein.

The present invention relates to polysaccharide adjuvants belonging to the class of C-type lectins (CLECs).

Vaccination is considered one of the most powerful means to save lives and to alleviate disease burden. By means of active immunization the vaccine is administered so that the immune system of the host develops a non-specific innate immune response as well as specific antibodies, B- and T memory cells that can act against the immunogen applied.

Mannan, a polysaccharide derived from the yeast cell wall, consists of a backbone of mostly β-(1,4)-linked mannose with a small number of α-(1,6)-linked glucose and galactose side chain residues. In addition, a protein content of approximately 5% has been detected in conventional mannan preparations. As an important component of fungal cell walls, mannan has been widely used as component in carbohydrate-based vaccines for Candidiasis (Han and Rhew, Arch Pharm Res 2012, Vol 35, No 11, 2021-2027; Cassone, Nat Rev Microbiol. 2013 December; 11(12):884-91; Johnson and Bundle, Chem. Soc. Rev., 2013, 42, 4327). In addition, different examples for vaccines based on mannan carrier-antigen complex/conjugations have been developed, including mannan-mucin 1 (MUC1) fusion protein conjugation for tumor therapy or conjugates of mannan and model allergens like ovalbumin (OVA), Papain or Betv1.

Mucins are heavily glycosylated proteins expressed on cell surfaces. MUC1 is a prototypical mucin, which has been found to be over-expressed on a wide range of tumor cells. Along these lines, a MUC1 Fusion Protein containing 5 tandem repeats of human MUC1 (containing the immune-dominant epitope: APDTRPAPGSTAPPAHGVTS) and peptide (Cpl3-32) were produced and conjugated to mannan under either oxidative or reductive conditions leading to drastically different immunological responses: oxidized mannan-MUC1 stimulated Th1 type responses mediated by CD8+ T-cells with IFN-γ secretion and mainly IgG2a antibody response, whereas reduced mannan-MUC1 stimulated Th2 type responses with IL-4 production and a high IgG1 antibody response. The employed fusion protein represented a single protein displaying T- and B-cell epitopes.

Recently also Protein-carbohydrate/mannan complexes of papain and OVA were generated to analyse their allergenic potential. It was found that coupling mannan to the protein surface could decrease binding and crosslinking of IgE antibodies directed against Papain. Interestingly, coupling of either mannan, dextran, or maltodextrin only reduced the allergenic potential of papain, but not OVA in these experiments indicating the importance of carbohydrate selection for vaccine design (Weinberger et al. J. Control. Release 2013; 165:101-109). These experiments also showed that mannan conjugation leads to the development of elevated IgG titers against OVA following intradermal immunization.

Similar to the neoglycoconjugate vaccine used for MUC1, Ghochikyan et al. (DNA AND CELL BIOLOGY, Volume 25, Number 10, 2006, Pp. 571-580) and Petrushina et al. (Journal of Neuroinflammation 2008, 5:42) were applying Amyloid beta (Aβ)28, a 28 aa residue peptide carrying combined B- and T-cell epitopes of the human Aβ42 peptide, coupled to mannan and could induce low levels of anti-Aβ responses in mice. These responses also proved to attenuate amyloid deposition in the cortical and hippocampal regions of APP transgenic mice following sub cutaneous immunization. The immunization also led to the induction of increased anti-mannan titers in Aβ28-mannan and BSA-mannan treated animals. The treatment however was not further developed most probably due to the occurrence of increased micro-haemorrhages in the brains of treated animals which were attributed to potential harmful effects of mannan as the trigger of adverse vascular events underlining the importance of carbohydrate selection for design of efficient and safe vaccines.

No conjugates using individual B-cell or T-cell epitope peptides coupled to mannan or other related CLECs are known so far.

β-Glucans comprise a group of β-D-glucose polysaccharides. These polysaccharides are major cell wall structural components in fungi and are also found in bacteria, yeasts, algae, lichens, and plants, such as oats and barley. Depending on the source, B-glucans vary in the type of linkage, the degree of branching, molecular weight and tertiary structure.

β-glucans are a source of soluble, fermentable fiber—also called prebiotic fiber—which provides a substrate for microbiota within the large intestine, increasing fecal bulk and producing short-chain fatty acids as by-products with wide-ranging physiological activities. For example, dietary intake of Cereal β-glucans from oat at daily amounts of at least 3 grams lowers total and low-density lipoprotein cholesterol levels by 5 to 10% in people with normal or elevated blood cholesterol levels.

Typically, β-glucans form a linear backbone with 1-3 β-glycosidic bonds but vary with respect to molecular mass, solubility, viscosity, branching structure, and gelation properties. Yeast and fungal β-glucans are usually built on a β-(1,3) backbone and contain β-(1,6) side branches, while cereal β-glucans contain both β-(1,3) and β-(1,4) backbone bonds with or without side branching.

β-Glucans are recognized by the innate immune system as pathogen-associated molecular patterns (PAMPs). The PRR dectin-1 has emerged as the primary receptor for these carbohydrates and β-glucan binding to dectin-1 induces a variety of cellular responses via the Syk/CARD9 signalling pathway, including phagocytosis, respiratory burst and secretion of cytokines. In addition, also complement receptor 3 (CR3, CD11b/CD18) has been implicated as receptor for β-glucans. It has been reported that the stimulation via dectin-1 primes Th1, Th17, and cytotoxic T lymphocyte responses.

Members of the β-glucan family include:

Beta-glucan peptide (BGP) is a high molecular weight (˜100 kDa), branched polysaccharide extracted from the fungus Trametes versicolor. BGP consists of a highly ramified glucan portion, comprising a β-(1,4) main chain and β-(1,3) side chain, with β-(1,6) side chains covalently linked to a polypeptide portion rich in aspartic, glutamic and other amino acids.

Curdlan is a high molecular weight linear polymer consisting of β-(1,3)-linked glucose residues from Agrobacterium spp.

Laminarin from the brown seaweed Laminaria digitata is a linear β-(1,3)-glucan with β-(1,6)-linkages. Laminarin is a low molecular weight (5-7 kDa), water-soluble β-glucan that can act either as a dectin-1 antagonist or agonist. It can bind to dectin-1 without stimulating downstream signalling and is able to block dectin-1 binding of particulate β-(1,3)-glucans, such as zymosan.

Pustulan is a median molecular weight (20 kDa), linear β-(1,6) linked β-D-glucan from lichen Lasallia pustulata which is also able to bind to dectin-1 as major receptor and activate signalling via dectin-1.

Lichenan is a high molecular weight (ca 22-245 kDa) linear, β-(1,3) β-(1,4)-β-D glucan from Cetraria islandica with a structure similar to that of barley and oat β-glucans. Lichenan has a much higher proportion of 1,3- to 1,4-β-D linkages than do the other two glucans. The ratio of β-(1,4)-to β-(1,3)-β-D linkages is approximately 2:1.

β-Glucan from oat and barley are linear, β-(1,3) β-(1,4)-β-D glucans and are commercially available with different molecular weights (medium molecular weight fractions of 35.6 kDa to high molecular weight fractions of up to 650 kDa).

Schizophyllan (SPG) is a gel-forming β-glucan from the fungus Schizophyllum commune. SPG is a high molecular weight (450 kDa) β-(1,3)-D-glucan that has a β-(1,6) monoglucosyl branch in every three β-(1,3)-glucosyl residues on the main chain.

Scleroglucan is a high molecular weight (>1000 kDa) polysaccharide produced by fermentation of the filamentous fungus Sclerotium rolfsii. Scleroglucan consists of a linear β-(1,3) D-glucose backbone with one β-(1,6) D-glucose side chain every three main residues.

Whole glucan particles (WGP) are beta-glucans notable for their ability to modulate the immune response. WGP Dispersible (WGP® Dispersible from Biothera) is a particulate Saccharomyces cerevisiae β-glucan preparation. It consists of hollow yeast cell wall “ghosts” composed primarily of long polymers of β-(1,3) glucose obtained after a series of alkaline and acid extractions from S. cerevisiae cell wall. In contrast to other dectin-1 ligands such as Zymosan, WGP Dispersible lacks TLR-stimulating activity. In contrast, soluble WGP binds dectin-1 without activating this receptor. And it can significantly block the binding of WGP Dispersible to macrophages and its immunostimulatory effect.

Zymosan, an insoluble preparation of yeast cell and activates macrophages via TLR2. TLR2 cooperates with TLR6 and CD14 in response to zymosan. Zymosan is also recognized by dectin-1, a phagocytic receptor expressed on macrophages and dendritic cells, which collaborates with TLR2 and TLR6 enhancing the immune responses triggered by the recognition of zymosan by each receptor.

As a major component of fungal cell walls, different β-glucans have been used as antigens for generating anti-glucan antibodies against fungal infections (e.g.: Torosantucci et al. J Exp Med. 2005 Sep. 5; 202 (5):597-606., Bromuro et al., Vaccine 28 (2010) 2615-2623, Liao et al., Bioconjug Chem. 2015 Mar. 18; 26(3):466-76).

Torosantucci et al. (2005) and Bromuro, et al. (2010) disclose conjugates of the branched β-glucan laminarin, and the linear β-glucan Curdlan coupled to the diphtheria toxoid CRM197. These conjugate vaccines induced high IgG titers against the β-glucan and conferred protection against fungal infections in mice. In addition, also high titers against CRM197 can be detected using such conjugates (Donadei et al., Mol Pharm. 2015 May 4; 12(5):1662-72). The authors have also generated β-glucan-CRM197 vaccines, with synthetic linear β-(1,3)-oligosaccharides or β-(1,6)-branched β-(1,3)-oligosaccharides, formulated with the human-acceptable adjuvant MF59. All conjugates induced high titers of anti-β-(1,3)glucan IgG and/or also anti-β-(1,6)-glucan antibodies in addition to the anti-β-(1,3)-glucan IgG demonstrating the immunogenicity of different glucans in combination with classical carrier proteins. Interestingly, Torosantucci et al. failed to demonstrate superior anti-CRM titers following immunization using CRM-glucan conjugates as compared to non-conjugated CRM alone.

Donadei et al. (2015) also analysed conjugates of the diphtheria toxoid CRM197 coupled to linear β-(1,3) glucan Curdlan or to synthetic β-(1,3) oligosaccharides. The conjugates were immunogenic, mounting comparable antibody responses against CRM197. Interestingly, the authors showed that CRM Curdlan conjugates when delivered intradermally resulted in higher antibody titers in comparison to intramuscular (i.m.) immunization. However, intradermal application of CRM-Curdlan did not show different immunogenicity as compared to sub cutaneous application. In addition, in vivo effects were comparable between CRM-Curdlan and non-Curdlan coupled CRM adjuvanted with Alum. Thus, no added benefit of the CLEC coupling on overall immune responses could be detectable in this system.

Liao et al. (2015) disclosed a series of linear β-(1,3)-β-glucan oligosaccharides (hexa-, octa-, deca-, and dodeca-β-glucans) which have been coupled to KLH to generate glycoconjugates. These conjugates were shown to elicit robust T-cell responses and were highly immunogenic inducing high anti-glucan antibody levels. Mice immunized with such vaccines were also eliciting protective immune responses against the deadly pathogen, C. albicans. No comparison of anti-KLH titers with non-conjugated KLH has been performed, hence no information on a potential benefit of the β-glucan is available in this experimental setting.

These findings are highly important for the applicability of glucan-based neoglycoconjugates as novel vaccines: potential anti-glucan antibodies induced upon an initial glucan-conjugate immunization could lead to quick elimination of either the same β-glucan vaccines in subsequent booster immunisations or could attenuate immune responses against novel neoglycoconjugate vaccines directed against other indications, an effect well known from vector vaccines. The presence or even (re)stimulation of high-level anti-glucan antibodies, as demonstrated above for mannan and β-glucans (Petrushina et al. 2008, Torosantucci et al. 2005, Bromuro et al., 2010, Liao et al., 2015), could thereby reduce or eliminate potential immune reactions elicited by conjugate vaccines. Thus, it would be crucial for a novel and sustainable platform using CLECs, especially β-glucans, as backbone for immunization to guarantee for a very low or absent glucan antibody inducing capacity of the poly/oligosaccharide used.

Glucan particles (GPs) are highly purified 2-4 μm hollow porous cell wall microspheres composed primarily of β-(1,3)-D-glucans, with low amounts of β-(1,6)-D-glucans and chitin, typically isolated from Saccharomyces cerevisiae, using a series of hot alkaline, acid and organic extractions. They interact with their receptors dectin-1 and CR3 (there is also evidence implying interaction with toll-like receptors and CD5 as additional factors for GP function) and upregulate cell surface presentation of MHC molecules, lead to altered expression of co-stimulation molecules as well as induce the production of inflammatory cytokines. Due to their immunomodulatory properties, GPs have been explored for vaccine delivery.

There are three general approaches for applying GPs in vaccines: (i) as a co-administered adjuvant with antigen(s) to enhance T- and B-cell-mediated immune responses, (ii) chemically crosslinked with antigens and most frequently used (iii) as a physical delivery vehicle of antigens trapped inside the hollow GP cavity, to provide targeted antigen delivery to APCs.

Ad (i): Antigen-specific adaptive immune responses can be enhanced by co-administering GPs together with antigens. In this conventional adjuvant strategy, both innate as well as adaptive immune responses are activated to exert protective responses against pathogens. Williams et al. (Int J Immunopharmacol. 1989; 11(4):403-10) for example adjuvanted a killed Trypanosoma cruzi vaccine by co-administering GPs. The immune response elicited using this formulation resulted in 85% survival of mice challenged with T. cruzi. In contrast, controls that received dextrose, glucan or vaccine alone had 100% mortality.

Ad (ii): The carbohydrate surface of GPs can also be covalently modified using NaIO₄ oxidation, carbodiimide cross-linking or 1-cyano-4-dimethylaminopyridinium tetrafluoroborate-mediated conjugation of antigens to the GP shell. Using this approach, coupling efficacies are very low (approx. 20%, e.g. as described in Pan et al. Sci Rep 5, 10687 (2015)), which limits applicability and the number of vaccine candidates significantly compared to i.e. antigen encapsulation in GPs or the proposed platform technology provided in this application. Such covalently linked antigen-GP conjugates were used in studies for cancer immunotherapy and infectious diseases. For example, Pan et al. (2015) used OVA cross-linked to periodate-oxidized GPs and subcutaneously immunized mice with this vaccine. When mice were challenged with OVA-expressing E.G7 lymphoma cells, a significant reduction in tumor size was observed. GP-OVA was detectable in DCs (CD11c⁺MHC-II⁺) in lymph nodes 12 and 36 h post-subcutaneous injection. Tumor protection was associated with an increase in total anti-Ova immunoglobulin (Ig)G titer, enhanced MHC-II and co-stimulatory molecule (CD80, CD86) expression and heightened cytotoxic lymphocyte responses.

Ad (iii): the most effective approach for applying GPs in vaccines is to employ them for encapsulation of vaccines/antigens into the hollow core. GPs can encapsulate one or more antigens/DNA/RNA/adjuvants/drugs/combinations thereof with high loading efficiency, which is dictated by the type of payload and the mode of delivery intended.

Antigens can be encapsulated in the hollow cavity of the GPs using polymer nano-complexation methods like loading and complexation of the payload using bovine or murine serum albumin and yeast RNA/tRNA or the addition of alginate-calcium or alginate-calcium-chitosan mixtures. Using these strategies, for example Huang et al. (Clin. Vaccine Immunol. 2013; 20:1585-91) reported that mice vaccinated with GP-OVA showed strong CD4+ T-cell lymphoproliferation, a Th1 and Th17 skewed T-cell-mediated immune response together with high IgG1- and IgG2c-specific antibody responses against ovalbumin. The non-covalent encapsulation strategy elicited stronger immune responses compared to GPs co-administered with antigen.

Examples for GP-encapsulated subunit vaccines are GPs encasing soluble alkaline extracts of Cryptococcus neoformans acapsular strain (cap59) which protected mice challenged with lethal doses of highly virulent C. neoformans (60% survival) by inducing an antigen-specific CD4+ T-cell response (positive for IFN-γ, IL-17A) that reduced the fungal colony-forming units (CFU) more than 100-fold from the initial challenge dose (Specht C A et al. Mbio 2015; 6: e01905-e1915. and Specht C A et al., mBio 2017; 8: e01872-e1917.). Additionally, vaccinating mice with GP encapsulating antigens proved efficacious against Histoplasma capsulatum (Deepe G S et al., Vaccine 2018; 36: 3359-67), F. tularensis (Whelan A O et al., PLOS ONE 2018; 13: e0200213), Blastomyces dermatitidis (Wuthrich M et al., Cell Host Microbe 2015; 17: 452-65) and C. posadasii (Hurtgen B J et al., Infect. Immun. 2012; 80: 3960-74).

Beside cancer and infectious disease applications, also a limited number of studies using self-antigens has been performed using GPs as encapsulation agent for vaccine delivery. Along these lines, Rockenstein et al. (J. Neurosci., Jan. 24, 2018 •38(4):1000-1014) describe the application of GPs loaded with recombinant human aSynuclein protein (containing both, B- and T-cell epitopes suitable for induction of anti-aSyn immune responses) and Rapamycin which is known to induce antigen-specific regulatory T-cells (Tregs) in a murine model for Synucleinopathies. As expected from previous studies using full length aSynuclein as immunogen, application of the GPs containing aSyn leads to induction of robust anti-aSynuclein antibody titers and alleviates aSynuclein triggered pathologic alteration in the animals to a similar extent as previously published. Addition of Rapamycin efficiently induced the formation of iTregs (CD25 and FOXP3+) cells as the number of such Treg cells was significantly increased following Rapamycin exposure. GPs loaded with antigen aSynuclein and Rapamycin were thus triggering both neuroprotective humoral and iTreg responses in mouse models of synucleinopathy with the combination vaccine (aSyn+Rapamycin) being more effective than either humoral (GP aSyn) or cellular immunization (GP rapamycin) alone. No information on comparability of the effects to conventional, non-GP containing aSynuclein immunization have been reported.

β-glucan neoglycoconjugates efficiently target dendritic cells via the C-type lectin receptor dectin-1, boosting their immunogenicity. Specifically, certain β-glucans have also been used as potential carriers for vaccination using model antigens like OVA (Xie et al., Biochemical and Biophysical Research Communications 391 (2010) 958-962; Korotchenko et al., Allergy. 2021; 76:210-222.) or fusion proteins based on MUC1 (Wang et al., Chem. Commun., 2019, 55, 253).

Xie et al. and Korotchenko et al. were using the branched B-glucan laminarin as backbone for OVA conjugation. These gluconeoconjugates were then applied to mice either epicutaneously or via the subcutaneous route. Xie et al. showed that laminarin/OVA conjugates but not non-conjugated mixing of the compounds was inducing increased anti-OVA CD4+ T-cell responses as compared to ovalbumin alone. Importantly, co-injection of unconjugated laminarin blocked this enhancement supporting the function of laminarin mediated APC targeting. As expected, native OVA and the mixture of OVA and laminarin stimulated low level of anti-OVA antibody production. On the contrary, OVA/laminarin conjugate significantly enhanced antibody responses. Similarly, Korotchenko et al. demonstrated that laminarin conjugation to OVA significantly increased uptake and induced activation of BMDCs and secretion of pro-inflammatory cytokines. These properties of LamOVA conjugate also resulted in enhanced stimulation of OVA-specific naive T-cells co-cultured with BMDCs. In a prophylactic immunization experiment the authors could confirm that immunization with LamOVA reduced its allergenicity and induced ˜threefold higher IgG1 antibody titers compared with OVA after two immunizations. However, this effect was lost in all groups treated after the third immunization when all groups displayed similar antibody titers. Lam/OVA conjugates and OVA/alum conjugates showed comparable therapeutic efficacy in a murine model of allergic asthma. Thus, these experiments could not provide a clearly superior effect of glucan-based conjugates compared to conventional vaccines.

Wang et al. (2019) analysed the effects of a β-glucan based MUC1 cancer vaccine candidate. Again, the MUC1 tandem repeat sequence GVTSAPDTRPAPGSTPPAH, a well-studied cancer biomarker, was chosen as the peptide antigen providing T- and B-cell epitopes within the repeat sequence. An ethylene glycol (i.e. PEG) spacer was used to link β-glucan and the MUC1 peptide with yeast β-(1,3)-β-glucan polysaccharide applying 1,1′-carbonyl-diimidazole (CDI)-mediated conditions. Size of the β-glucan-MUC1 nanoparticles have been in the range of 150 nm (actual average 162 nm) while unmodified β-glucan was forming particles of approx. 540 nm. The β-glucan-MUC1 conjugate elicited high titers of anti-MUC1 IgG antibodies, significantly higher compared to the control groups. Further analysis of the isotypes and subtypes of the antibodies generated showed that IgG2b is the major subtype, indicating the activation of Th1-type response as a ratio of IgG2b/IgG1 is >1. The observed substantial amount of IgM antibodies indicates the involvement of the C3 component of the complement system, which often induces cytotoxicity and could be problematic for use of such backbones for vaccines which should avoid the development of cytotoxicity, e.g. for chronic or degenerative diseases.

US 2013/171187 A1 discloses an immunogenic composition comprising a glucan and a pharmaceutically acceptable carrier to elicit protective anti-glucan antibodies. Metwali et al. (Am. J. Respir. Crit. Care Med. 185 (2012), A4152; poster session C31 Regulation of Lung Inflammation) investigate into the immunomodulatory effect of a glucan derivative in lung inflammation. WO 2021/236809 A2 discloses a multi-epitope vaccine comprising amyloid-beta and tau peptides for the treatment of Alzheimer's disease (AD). US 2017/369570 A1 discloses β-(1,6)-glucan linked to an antibody directed to a cell present in a tumor microenvironment. US 2002/077288 A1 discloses synthetic immunogenic but non-amyloidogenic peptides homologous to amyloid-beta alone or conjugated for the treatment of AD. US 2013/171187 A1 discloses anti-glucan antibodies used as protective agents against fungal infections with C. albicans. WO 2004/012657 A2 discloses a microparticulate β-glucan as a vaccine adjuvant. CN 113616799 A discloses a vaccine vector consisting of oxidized mannan and a cationic polymer. CN 111514286 A discloses a Zika virus E protein conjugate vaccine with a glucan. U.S. Pat. No. 4,590,181 A discloses a viral antigen mixed in solution with pustulan or mycodextran. Lang et al. (Front. Chem. 8 (2020): 284) reviews carbohydrate conjugates in vaccine developments. Larsen et al. (Vaccines 8 (2020): 226) report that pustulan activated chicken bone marrow-derived dendritic cells in vitro and promotes ex vivo CD4+ T-cell recall response to infectious bronchitis virus. US 2010/266626 A1 discloses glucans, preferably laminarin and curdlan, as antigens conjugated to an adjuvant for immunising against fungi. Mandler et al. (Alzh. Dement. 15 (2019), 1133-1148) report on the effects of single and combined immunotherapy approach targeting amyloid-beta protein and alpha synuclein in a dementia with Lewy bodies-like model. Mandler et al. (Acta Neuropathol. 127 (2014), 861-879) reports a next-generation active immunization approach for synucleinopathies using short, immunogenic (B-cell response) peptides that are too short for inducing a T-cell response (autoimmunity) and do not carry the native epitope, but rather a sequence that mimics the original epitope (e.g., oligomeric alpha synuclein) and its implications for Parkinson's disease (PD) clinical trials. Mandler et al. (Mol. Neurodegen. 10 (2015), 10) report that active immunization against alpha synuclein ameliorates the degenerative pathology and prevents demyelination in a model of multiple system atrophy (MSA). Jin et al. (Vaccine 36 (2018), 5235-5244) review β-glucans as potential immunoadjuvants, mainly on the adjuvanticity, structure-activity relationship and receptor recognition properties. WO 2022/060487 A1 discloses a vaccine comprising specific alpha synuclein peptides for the treatment of neurodegenerative diseases. WO 2022/060488 A1 discloses a multi-epitope vaccine comprising amyloid-beta and alpha synuclein peptides for the treatment of AD. US 2009/169549 A1 discloses conformational isomers of modified versions of alpha synuclein produced by introducing cysteines into the alpha synuclein polypeptide and scrambling the disulphide bonds to form stable and immunogenic isomers. WO 2009/103105 A2 discloses vaccines with mimotopes of the alpha synuclein epitope extending from amino acid D115 to amino acid N122 in the native alpha synuclein sequence.

So far, no reports have been published demonstrating the construction or use of individual B-cell or T-cell epitope peptides which were coupled to β-glucans, especially linear β-glucans and/or pustulan with high binding specificity/ability to dectin-1, thereby forming novel gluconeoconjugates as those proposed in this application.

It is therefore an object of the present invention to provide improved vaccines as conjugate vaccines made of the vaccination antigen conjugated to carbohydrate-based CLEC adjuvants, especially to provide vaccines which provide an improved immune response in the vaccinated individual compared to current state of the art conjugate vaccines, especially carbohydrate-based CLEC-peptide/protein conjugate vaccines.

It is a further object of the present invention to provide vaccine compositions which confer immunity to short, easily interchangeable, highly specific B/T-cell epitopes using a CLEC backbone with previously unmet efficacy, specificity and affinity by conventional vaccines.

A specific object of the present invention is the provision of vaccines with improved selectivity and/or specificity of a CLEC-based vaccine for the dermal compartment.

Another object of the present invention is to provide vaccines which—as exclusively as possible—induce target-specific immune responses while inducing no or only very limited CLEC- or carrier protein-specific antibody responses.

It is a further object of the present invention to provide vaccine compositions which confer immunity to short, easily interchangeable, highly specific B/T-cell epitopes of alpha synuclein using a CLEC backbone with previously unmet efficacy, specificity and affinity by conventional vaccines for appropriate prevention and treatment of synucleopathies.

A specific object of the present invention is the provision of alpha synuclein vaccines with improved selectivity and/or specificity of a CLEC-based vaccine for the dermal compartment.

Another object of the present invention is to provide vaccines which—as exclusively as possible—induce alpha synuclein—specific immune responses while inducing no or only very limited CLEC- or carrier protein-specific antibody responses.

Another object of the present invention is to provide peptide immunogen constructs of the alpha synuclein protein (aSyn) and formulations thereof for treatment of synucleinopathies.

Therefore, the present invention provides a conjugate consisting of or comprising at least a β-glucan or a mannan and at least a B-cell or T-cell epitope polypeptide, wherein the β-glucan or mannan is covalently conjugated to the B-cell and/or T-cell epitope polypeptide to form a conjugate of the β-glucan or mannan and the B-cell and/or T-cell epitope polypeptide and wherein the B-cell and/or a T-cell epitope polypeptide is an alpha synuclein polypeptide. Alternatively, the conjugate according to the present invention consists of or comprises at least a β-glucan or a mannan and at least an alpha synuclein B-cell epitope polypeptide, wherein the β-glucan or mannan is covalently conjugated to the B-cell epitope polypeptide to form a conjugate of the β-glucan or mannan and the B-cell epitope polypeptide.

Preferably, the β-glucan is pustulan, lichenan, laminarin, curdlan, β-glucan peptide (BGP), schizophyllan, scleroglucan, whole glucan particles (WGP), zymosan, or lentinan, preferably pustulan, laminarin, lichenan, lentinan, schizophyllan, or scleroglucan, especially wherein the β-glucan is pustulan.

According to a preferred embodiment of the present invention, the β-glucan is for use as a C-type lectin (CLEC) polysaccharide adjuvant for B-cell and/or T-cell epitope polypeptides, especially wherein the β-glucan is covalently conjugated to the B-cell and/or T-cell epitope polypeptide to form a conjugate of the β-glucan and the B-cell and/or T-cell epitope polypeptide, wherein the β-glucan is a predominantly linear β-(1,6)-glucan with a ratio of β-(1,6)-coupled monosaccharide moieties to non-β-(1,6)-coupled monosaccharide moieties of at least 1:1, preferably at least 2:1, more preferred, at least 5:1, especially at least 10:1.

With the present invention one or more objects listed above are successfully solved. This was unexpected for a person skilled in the art, because until now, no reports have been published in the present field of technology demonstrating the construction and applicability or efficacy of compounds similar to the novel, small, modular gluconeoconjugates according to the present invention.

Surprisingly, it was shown with the present invention that by conjugation (i.e. by covalently coupling; used synonymously herein) of peptides/proteins to the selected CLEC-carrier according to the present invention, wherein the conjugation may be based on state-of-the-art chemistry, superior pharmaceutical formulations for effecting immune responses were obtained. In the present field of technology, a significant number of different coupling methods is available. In the course of establishing the present invention, hydrazone formation or coupling via heterobifunctional linkers have been identified as specifically preferred methods. In general, activation of the CLEC prior to conjugation (e.g.: formation of reactive aldehydes on vicinal OH groups of the sugar moieties) and presence of reactive groups on the peptide/protein of choice (e.g. N- or C-terminal hydrazide residues, SH groups (e.g.: via N- or C-terminal cysteines)) is required. The reaction can be a single step reaction (e.g. mixing of activated CLECs with Hydrazide-peptides leading to hydrazone formation or a multistep process (e.g.: activated CLEC is reacted with a hydrazide from a heterobifunctional linker and subsequently the peptide/protein is coupled via respective reactive groups).

Accordingly, the components of the conjugates according to the present invention may be directly coupled to each other, e.g. by coupling the B-cell epitope and/or the T-cell epitope to the β-glucan or mannan and/or to a carrier protein or by coupling the β-glucan or mannan to a carrier protein (in all possible orientations). Referring to a “B-cell epitope polypeptide” or a “T-cell epitope polypeptide” herein means by default the B-cell or T-cell epitope of the “B-cell epitope polypeptide” or the “T-cell epitope polypeptide” and not to a B-cell or T-cell epitope of the carrier protein (if present), except if it is explicitly referred to a B-cell or T-cell epitope of the carrier protein. According to a preferred embodiment, the B-cell epitope and/or the T-cell epitope is preferably linked to the β-glucan or mannan and/or to a carrier protein by a linker, more preferred a cysteine residue or a linker comprising a cysteine or glycine residue, a linker resulting from hydrazide-mediated coupling, from coupling via heterobifunctional linkers, such as N-β-maleimidopropionic acid hydrazide (BMPH), 4-[4-N-maleimidophenyl]butyric acid hydrazide (MPBH), N-[ε-Maleimidocaproic acid) hydrazide (EMCH) or N-[κ-maleimidoundecanoic acid]hydrazide (KMUH), from imidazole mediated coupling, from reductive amination, from carbodiimide coupling a —NH—NH₂ linker; an NRRA, NRRA-C or NRRA-NH—NH₂ linker, peptidic linkers, such as bi-, tri-, tetra- (or longer)-meric peptide groups, such as CG or CG, or cleavage sites, such as a cathepsin cleavage site; or combinations thereof, especially by a cysteine or NRRA-NH—NH₂ linker.

It is clear that “a linker resulting from (e.g.) hydrazide-mediated coupling” refers to the resulting chemical structure in the conjugate after conjugations, i.e. as present in the resulting conjugate after conjugation. Amino acid linkers may be present in the conjugated form either with a peptidic bond (e.g. with glycine containing linkers) or via a functional group of the amino acid (such as the disulfide bond for cysteine linkers).

The novel class of conjugates according to the present invention turned out to confer immunity to short, easily interchangeable, highly specific B/T-cell epitopes by using the CLEC backbone of the present invention showing efficacy, specificity and affinity previously unmet by conventional vaccines: In fact, the conjugates according to the present invention are the first examples for use of short B-cell/T-cell epitopes in a CLEC based vaccine avoiding the need for presenting the alpha synuclein epitopes in the form of fusion proteins including formation of tandem repeats of epitopes or fusion of different tandem repeats to form a stable and effective immunogen.

With the present invention, also the necessity to use full length proteins for use in CLEC vaccines (i.e. payload in glucan particles (GPs)) can be avoided. Moreover, the problem of autoimmune-reactions especially induced by (unwanted) T-cell epitopes present in immunogens like self-proteins such as alpha synuclein can also be avoided.

According to the present invention, for the first-time short epitopes (B- and/or T-cell epitopes, mainly peptides, modified peptides) can be united with a functional CLEC-based backbone using covalent coupling based on well-established chemistry wherein the possible methods for conjugation can be adapted to the requirements of the specific epitope based on methods well known in the field.

The presentation of the short peptide(s) according to the present invention can be made as individually conjugated moieties in combination with an individual foreign T-cell epitope (as short peptide or long protein) or as a complex/conjugate with a larger carrier molecule providing the T-cell epitope for inducing a sustainable immune response. The design of the vaccines according to the present invention allows for preparation of multivalent conjugates as a prerequisite for efficient immune response induction by highly efficient B-cell receptor (BCR)-crosslinking.

Moreover, with the present invention a CLEC based vaccine can be provided with an excellent selectivity/specificity for the dermal compartment. In fact, the conjugate design according to the present invention builds on CLECs as carrier for the target specific alpha synuclein epitopes which display high binding specificity for PRRs on dermal APCs/DCs, especially on dectin-1 (or MR and DC-SIGN in the case of mannan) to allow for skin selective/specific- and receptor mediated uptake (=targeted vaccine delivery).

The CLEC polysaccharide used as carrier according to the present invention is used to focus the carrier-peptide conjugate into preferably dermal/cutaneous DCs and to initiate an immune response. This is i.a. due to an epidermal or dermal (not sub-cutaneous) specificity. The CLEC backbone and the efficient dermal immune response initiation according to the present invention also helps to avoid the compulsory use of adjuvants, typical for conventional vaccines and also used in exemplary CLEC based vaccines (e.g.: use of Alum, MF59, CFA, PolyI:C or other adjuvants). According to a preferred embodiment of the present invention, the use of adjuvants may be significantly reduced or omitted, e.g. in circumstances wherein addition of adjuvants is not indicated.

Several CLECs have been used in previous applications however none of the proposed conjugate structures could confer skin selectivity (i.e. high dectin-1 binding ability, highly efficient dermal DC targeting and superior immunogenicity for dermal application as compared to all other routes (i.e. subcutaneous, intramuscular and i.p.).

The selection of the CLEC according to the present invention has been made as to provide a novel solution to target skin specific DCs and skin specific immunization with high efficacy. The conjugates according to the present invention also exert limited activity in other classical tissues for immunization like muscle or sub-cutaneous tissue which is in contrast to previous CLEC-based vaccines/vaccine candidates described which have been applied i.m. or s.c. As a result of the experiments conducted in the course of the present invention, vaccines according to the present invention, especially those which use pustulan as CLEC were identified as being surprisingly selective for skin immunization.

With the present invention, it becomes possible to focus on the induction of alpha synuclein specific immune responses while inducing no or only very limited CLEC- or carrier-protein specific antibody responses. The conjugates according to the present invention thereby solve the problem posed by classical conjugate vaccines, which have to rely on the use of foreign carrier proteins to induce a sustainable immune response. Current state of the art conjugate vaccine development is strongly built on carrier molecules like KLH, CRM197, Tetanus Toxoid or other suitable proteins, which are complexed with target specific short antigens delivering the substrate for immune reactions against alpha Synuclein for synucleinopathies like Parkinson's disease.

Preferred polypeptide immunogen constructs according to the present invention contain a B-cell epitope from alpha synuclein (aSyn, alpha Syn) and a heterologous T helper cell (Th) epitope coupled to a CLEC. The present invention delivers surprisingly superior new conjugates which are surpassing conventional vaccines in immunogenicity, cross reactivity against aSyn, selectivity for aSyn species/aggregates, affinity, affinity maturation and inhibition capacity as compared to conventional vaccines.

The covalent conjugation of the alpha synuclein polypeptide to the β-glucan or mannan according to the present invention enables a surprisingly and unexpectable enhancement of the immune response for such polypeptides. This is specifically impressive in direct comparison with traditional vaccine formulations, such as the ones described by Rockenstein et al. (2018), as also demonstrated in the example section below.

Rockenstein et al. (2018) disclose the application of yeast whole glucan particles (GPs) non-covalently complexed with aSyn and rapamycin as immunotherapeutic for Parkinson's disease. These GPs have been created following a series of hot alkali, organic, and aqueous extraction steps from Saccharomyces cerevisiae leading to the final product consisting of a highly purified 3- to 4-μm-diameter yeast cell wall preparations devoid of cytoplasmic content and bounded by a porous, insoluble shell of β-glucans (mainly β1-3 β-glucans).

Importantly, the vaccine composition disclosed by Rockenstein et al. (J. Neurosci., Jan. 24, 2018 • 38(4):1000-1014) consisted of GPs which were non-covalently complexed with either ovalbumin and mouse serum albumin (MSA), human aSyn and MSA or human aSyn, MSA and rapamycin. This complexation method relies on co-incubation of the different payloads with GPs and the subsequent diffusion into the hollow GP cavity without covalent attachment and is therefore similar to a set of vaccines disclosed in Example 5 provided within this application where only a mixing but no covalent attachment of components was used to formulate a vaccine and which proved inefficient and unsuitable as compared to the vaccines according to the present invention.
1) Rockenstein et al. show that non-covalent mixing of aSyn and GPs leads to a detectable immune response against aSyn hence showing that GPs can act as adjuvants. However, Rockenstein et al. also show that the non-covalent addition/co-complexation of rapamycin is required to induce significantly enhanced functionality of such a vaccine as compared to controls. In this view a mix of various adjuvants (GPs as well as the mTOR inhibitor rapamycin) is required to provide a fully functional vaccine, such as the vaccines disclosed by the present invention.
2) The vaccine disclosed by Rockenstein et al. is active in this aSyn overexpression model as it provides aSyn specific T-cell epitopes (among other T-cell epitopes like MSA-derived epitopes) in order to exert its full functionality namely induction of a neuroprotective, anti-aSyn directed cellular (i.e.: T-cell mediated) and humoral (i.e. antibody/B-cell based) immune response. This is in direct contrast to the teachings of the present invention, where it is already sufficient if only aSyn specific B-cell responses are elicited by the vaccines selected.
3) The use of full length aSyn is also posing the danger of inducing/augmenting autoreactive, aSyn specific T-cells which have the potential to exacerbate the underlying neuropathology in PD and other synucleopathies. Hence the GP-aSyn-rapamycin vaccine proposed by Rockenstein et al. is also with respect to this issue not preferred for human use.
4) As shown in Example 5 non-covalent mixing of aSyn derived peptides (e.g.: SeqID2 i.e. B-cell epitopes) and promiscuous T-cell epitopes (e.g.: SeqID7) with a β-Glucan particle (e.g.: non-oxidised pustulan), similar to Rockenstein et al., is also able to induce a low level antibody response against aSyn. However, vaccines according to the present invention, which build on covalent linkage of such peptides to a suitable glucan exert a significantly different and superior immune response (see also FIG. 5).

In addition, and also disclosed in Example 6 and FIG. 7, such covalently linked vaccines also show a highly beneficial lack of anti-glucan antibody responses as compared to non-covalently mixed vaccines building on glucan particles and peptides as disclosed by the present invention.

Hence, the prior art disclosure by Rockenstein et al. does not suggest the claimed subject matter disclosed by the present invention.

Any alpha-synuclein polypeptide comprising a B-cell and/or a T-cell epitope is useable in the context of the present invention including the polypeptide vaccine candidates proposed in the prior art, e.g. those disclosed in WO 2004/041067 A2, WO 2006/045037 A2, WO 2009/103105 A2, WO 2011/020133 A1, or in Mandler et al. (Mol. Neurodeg. 10 (2015), 10; Acta Neuropath. 127 (2014), 861-879).

Accordingly, alpha-synuclein polypeptides with native amino acid sequence (according to human alpha-synuclein) or aSyn-derived polypeptides comprising a B-cell and/or a T-cell epitope of aSyn, such as mimics or mimotopes thereof can be used as alpha-synuclein polypeptide component in the conjugates according to the present invention.

Specifically preferred aSyn polypeptides to be conjugated in the present invention are selected from native alpha synuclein or a polypeptide comprising or consisting of amino acid residues 1 to 5, 1 to 8, 1 to 10, 60 to 100, 70 to 140, 85 to 99, 91 to 100, 100 to 108, 102 to 108, 102 to 109, 103 to 129, 103 to 135, 107 to 130, 109 to 126, 110 to 130, 111 to 121, 111 to 135, 115 to 121, 115 to 122, 115 to 123, 115 to 124, 115 to 125, 115 to 126, 118 to 126, 121 to 127, 121 to 140, or 126 to 135, of the amino acid sequence of native human alpha synuclein: MDVFMKGLSK AKEGVVAAAE KTKQGVAEAA GKTKEGVLYV GSKTKEGVVH GVATVAEKTK EQVTNVGGAV VTGVTAVAQK TVEGAGSIAA ATGFVKKDQL GKNEEGAPQE GILEDMPVDP DNEAYEMPSE EGYQDYEPEA (human aSyn (1-140 aa): UNIPROT accession number P37840),

preferably a polypeptide comprising or consisting of amino acid residues 1 to 8, 91 to 100, 100 to 108, 103 to 135, 107 to 130, 110 to 130, 115 to 121, 115 to 122, 115 to 123, 115 to 124, 115 to 125, 115 to 126 118 to 126, 121 to 127, or 121 to 140; or mimotopes selected from the group DQPVLPD, DQPVLPDN, DQPVLPDNE, DQPVLPDNEA, DQPVLPDNEAY, DQPVLPDNEAYE, DSPVLPDG, DHPVHPDS, DTPVLPDS, DAPVTPDT, DAPVRPDS, and YDRPVQPDR.

Weihofen et al. (Neurobiology of Disease 124 (2019) 276-288) disclosed N-terminal residues aa1-10 (MDVFMKGLSK) as a suitable aSyn epitope. PRX002 (as disclosed in Masliah et al., (PLoS One 6:e19338); Games et al., (2014, J Neurosci 34:9441-9454); Schenk et al., (Mov Disord. 2017 February; 32(2):211-218.); Jankovic et al., (JAMA Neurol. 2018 Oct. 1; 75(10):1206-1214.)) is directed to the C-terminal region aa118-126 (VDPDNEAYE). Schofield et al. (Neurobiol Dis. 2019 December; 132:104582.) disclosed the aSyn specific monoclonal Ab MEDI-1341, directed against a similar epitope in the C-terminal region of aSyn (aa 103-129, NEEGAPQEGILEDMPVDPDNEAYEMP). MEDI Ab (aa121-127; DNEAYEM) was disclosed by Nordstram et al., (Neurobiology of Disease 161 (2021) 105543). Other suitable epitopes for antibodies/immunotherapeutics in aSyn known to the person skilled in the art include epitopes from autoantibodies (as disclosed in Heinzel et al., (PLoS ONE 9(12): e114566.); Rabenstein et al., (Neurosci Lett. 2019 Jun. 21; 704:181-188); US 2014/0377271 A1 and Li et al., (Acta Neuropathologica 137, 825-836 (2019)) include aa60-100, aa60-95, aa73-82, aa91-100; aa74-79 and aa92-97, aa121-140 and aa111-121. In addition, Games et al. (2014) also disclose suitable epitopes aa 91-99 and aa 118-126. In US 2005/0037013 A1, immunogenic alpha-synuclein fragments are disclosed which are able to induce an immune response against a specific epitope within residues aa70-140. WO 2009/103105 A2 and WO 2011/020133 A1 disclose molecular mimics to aa102-108 and aa115-121, respectively. WO 2016/062720 A1 provides modified VLPs comprising Th cell epitopes representing the middle region (aa102-109), as well as N-terminal (aa1-8) sequences or C-terminal sequences (i.e. aa131-140) which were able to induce a high anti-peptide response. Middle and C-terminal derived epitopes were also able to induce aSyn specific titers recognizing different aSyn species (e.g.: monomeric and oligomeric). Interestingly, the vaccines provided all failed in reducing aSyn burden or behavioural alterations in in vivo PD models. US 2015/0232524 A1 discloses polypeptide immunogens, especially aa10-14 and aa36-69 and epitopes were shown in aa85-99, aa109-126 and aa126-140. Interestingly, all constructs mount an anti-aSyn immune reaction however epitope aa109-126 seems less effective. WO 2018/232369 A1 shows the application of target specific B-cell epitopes (10-25aa, derived from the C-terminus of aSyn; region: aa111-135).

Although in principle, the present invention is able to improve all suggested aSyn vaccination polypeptides, selected epitopes were specifically assessed with respect to their suitability with the present platform. For example, aa1-8 (SeqID12+13) were shown to be superior to a KLH based vaccine.

Whereas the nature of the alpha synuclein polypeptide is not critical for the β-glucan or mannan conjugate generation, there are preferred and less preferred alpha synuclein epitopes, depending on the object the conjugate has to solve (specificity for monomers or aggregates, selectivity, affinity, generation of antibodies, etc.).

For some embodiments of the present invention, B-cell epitopes aa91-100, aa100-108, aa107-114 and aa131-140 are less preferred due to decreased anti-peptide response and decreased reactivity to alpha synuclein (aa91-100); low cross-reactivity (CR) to alpha synuclein (despite high anti-peptide titer), lower selectivity for aggregates (aa100-108); less potency to inhibit aggregation (aa107-114); and change of selectivity (for monomer instead of aggregates), reduced selectivity, less effectivity for lowering alpha synuclein aggregates (aa131-140). Moreover, epitope polypeptides with 5 or less amino acid residues (or with 6 or less amino acid residues) are usually also less preferred due to lower immune responses to be elicited with such short forms.

Specifically preferred alpha synuclein epitopes according to the present invention comprise the above mentioned epitopes comprising or consisting of amino acid residues 1 to 5, 1 to 8, 1 to 10, 60 to 100, 70 to 140, 85 to 99, 91 to 100, 100 to 108, 102 to 108, 102 to 109, 103 to 129, 103 to 135, 107 to 130, 109 to 126, 110 to 130, 111 to 121, 111 to 135, 115 to 121, 115 to 122, 115 to 123, 115 to 124, 115 to 125, 115 to 126, 118 to 126, 121 to 127, 121 to 140, or 126 to 135, of the amino acid sequence of native human alpha synuclein.

For example, aa115-126 and the shorter sequences and mimotopes thereof mentioned above are specifically suitable with respect to immunogenicity, selectivity, aggregate inhibition, etc.; generally, polypeptides with 7 or more amino acid residues show good immune response and cross-reactivity to alpha synuclein; for aa 115-121 (and the polypeptides extended C-terminally (e.g. until aa126) show excellent immune reactivity and cross-selectivity (even in contrast to the monoclonal LB509 which binds to epitope 115-122 (Jakes et al., Neurosci Lett. 1999 Jul. 2; 269(1):13-6)); surprisingly, the conjugates according to the present invention show a significantly improved selectivity for aggregates compared to “classical constructs” using CRM197 or KLH as carrier protein (whereas e.g. CRM-based vaccines frequently lack selectivity for aggregates or are even selective for the monomer, the conjugates according to the present invention show clear and pronounced activity towards aggregates).

This is even more surprising in view of the published experiments where LB509 binds to alpha synuclein aggregates but is not able to inhibit aggregation (Breydo et al. Mol Neurobiol 53, 1949-1958 (2015)) which shows that not every antibody from this epitope is biologically active as in case of the present invention.

It is well accepted in the field that PD patients suffer from different changes in their T-cell compartment as compared to healthy controls (e.g.: Bas et al., J Neuroimmunol 2001; 113:146-52 or Gruden et al., J Neuroimmunol 2011; 233:221-7). Such phenotypic changes of T-cells in PD are for example: reduced absolute lymphocyte counts, decreased absolute and relative counts of total T-cells, decreased absolute and relative counts of CD4+, and sometimes also CD8+ lymphocytes, increased Th1/Th2 and Th17/Treg ratios and increased expression of inflammatory cytokines. However, most of these changes are also found during healthy aging, making it difficult to discern the impact of a disease, such as PD, which presents with a very broad range of onset (˜30-90 years) and variable progression rate. Regarding absolute cell numbers, there appears to be consensus of a net reduction in CD3+CD4+ T-cells in PD. This CD4 reduction is supported by the altered CD4:CD8 ratio described.

Along these lines, for example, Bhatia et al. (J Neuroinflammation (2021) 18:250) show an overall decrease of total CD3+ T-cells in PD associated with disease severity (e.g. measured using H+Y stages). This suggests a progressing generalized T-cell dysfunction with ongoing disease, probably reflecting the combined effect of ongoing inflammation, medication and lifestyle change. Also, Lindestam Arlehamn et al. (2020) show that highest T-cell activity is detectable in PD patients at prodromal or early clinical stages (<10 years duration and H+Y stages 0-2).

Thus, it is a preferred embodiment of the present invention to provide a treatment for augmenting or preserving T-cell numbers, especially T-effector cell numbers, and T-cell function in a PD patient. This preferably includes a combination of checkpoint inhibitors or vaccines using anti-immune check point inhibitor epitopes to induce an anti-immune checkpoint inhibitor immune response in combination with target specific vaccines of the current invention to augment or preserve T-cell numbers, especially T-effector cell numbers and T-cell function in a PD patient.

Patients amenable to/suitable for the treatment are characterized by an overall reduction of CD3+ cells, especially of CD3+CD4+ cells typical for PD patients at all stages of the disease. The preferred stages of disease defining the suitable patient groups for this combination are H+Y stages 1-4, preferred H+Y1-3, most preferred H+Y 2-3, respectively.

Also affinity maturation of target specific responses induced upon repeated immunization using carrier conjugates is compromised due to overrepresentation of carrier specific epitopes in the conjugates. Affinity maturation in immunology, as used and understood herein, is the process by which T_{FH} cell-activated B-cells produce antibodies with increased affinity for antigen during the course of an immune response. With repeated exposures to the same antigen, a host will produce antibodies of successively greater affinities. A secondary response can elicit antibodies with several fold greater affinity than in a primary response. Affinity maturation primarily occurs on surface immunoglobulin of germinal center B-cells and as a direct result of somatic hypermutation (SHM) and selection by T_{FH} cells (see also: https://en.wikipedia.org/wiki/Affinity_maturation). Affinity Maturation according to the Segen's Medical Dictionary (https://medical-dictionary.thefreedictionary.com/affinity+maturation”>affinity maturation</a>) is the increased average affinity of antibodies to an antigen, which follows immunisation. Affinity maturation results from an increase of specific and more homogeneous IgG antibodies, and follows a less specific and more heterogeneous early response by IgM molecules.

The current state of the art CLEC vaccines all induce high titers against the carrier proteins used (e.g.: CRM197 or OVA). However, this high immunogenicity as well as the structural complexity and heterogeneity of the carrier protein component may lead to the induction of high levels of carrier/protein specific antibodies at the expense of target specific responses which therefore might be underrepresented in comparison to the carrier response induced.

Furthermore, high anti-carrier responses also pose the risk of immunological rejection and associated safety issues.

Thus, the identification of effective constructs with high immunogenicity, high target specificity and high tolerability/safety with low or absent carrier reactivity (i.e. against the protein carrier) according to the present invention successfully addresses this challenge by innovative solutions. In addition, it is crucial for the novel vaccines according to the present invention to provide immunotherapeutic agents which are inducing no/very weak immune responses against the sugar backbone. This is especially important as high anti-CLEC antibody levels induced upon immunization could inhibit or lower the efficacy of repeated immunization using the same CLEC-based vaccine due to vaccine neutralization or could also negatively impact the use of this type of vaccines for consecutive immunization against various different targets.

The vaccine platform according to the present invention also fulfils the need to combine various epitopes directed to one or several targets within one formulation without posing the risk to reduce efficacy due to unintended epitope spreading as reported for classical vaccines. The modular design of the platform according to the present invention allows for easy exchange of B- and T-cell epitopes without negative effects of a carrier induced response.

The present invention is based on a CLEC which exerts high specific binding to the cognate receptor. This binding is crucial and only strong binders are efficient as vaccine carriers/backbones.

According to the present invention, CLEC-conjugation enables an efficient immune response with novel characteristics. The conjugation according to the present invention precludes formation of anti-CLEC antibodies, especially for pustulan, such preclusion could be impressively shown in the course of the present invention. This lack of elicitation of anti-CLEC antibodies is very important for reusability and for reboostability of individual vaccines designed with the platform according to the present invention—be it with the same or different antigens.

In contrast to the conjugated embodiment of the present invention, a mere mixing of the CLEC polysaccharide adjuvant and the B-cell or T-cell epitope peptides does not lead to comparable effects in vivo. If conjugated, however, orientation of the peptide does not significantly influence the performance of the compounds according to the present invention; CLEC conjugation is therefore substantially independent from peptide orientation in the construct. In the course of the present invention, it could be shown that CLEC conjugation, especially to pustulan, leads to improvement of novel as well as existing peptide immunogens/antigens of alpha synuclein: This improvement is effected by higher, more target specific and more affine antibody reactions (as can be shown by antibody selectivity and functionality). This effect is most pronounced for pustulan or similar β-glucans which are predominantly linear β-(1,6)-glucans with a ratio of β-(1,6)-coupled monosaccharide moieties to non-β-(1,6)-coupled monosaccharide moieties of at least 1:1, preferably at least 2:1, more preferred, at least 5:1, especially at least 10:1, which performed surprisingly even considerably better than KLH or CRM in direct comparison and even better than mannan or lichenan conjugates or conjugates comprising barley β-glucans.

As used herein, the term “predominantly linear” β-(1,6)-glucans refers to β-(1,6)-D-glucans where no or only few cross-linking sugar monomer entities are present, i.e. wherein less than 1%, preferably less than 0.1%, especially less than 0.01%, of the monosaccharide moieties have more than two covalently attached monosaccharide moieties.

As already stated above, pustulan is the most preferred CLEC according to the present invention. Pustulan is usually free of cross-linking sugar moieties and predominantly β-(1,6)-coupled so that usual pustulan preparations to be used in the preparation of the conjugates according to the present invention contain less than 1%, preferably less than 0.1%, especially less than 0.01%, monosaccharide moieties with more than two covalently attached monosaccharide moieties, and contains maximally 10% impurities with β-(1,3)- or β-(1,4)-coupled monosaccharides.

The fact that pustulan turned out to be the most effective CLEC in the course of the present invention was unexpected, because various references show that pustulan should be less effective in Dectin-1 binding (e.g. Adams et al., J Pharmacol Exp Ther. 2008 April; 325(1):115-23); in the literature, linear 1,3 and branched (1,3 main chain and 1,6 side branch) have been reported to be the most effective Dectin-1 binders. For example, Adams et al., 2008, have reported that murine recombinant Dectin-1 only recognized and interacted with polymers that contained a β-(1,3)-linked glucose backbone. Dectin-1 did not interact with a glucan that was exclusively composed of a β-(1,6)-glucose backbone (pustulan), nor did it interact with non-glucan carbohydrate polymers, such as mannan.

Therefore, according to a preferred embodiment of the present invention, the β-glucan of the present conjugate is a dectin-1 binding β-glucan. The ability of any compound, especially glucans, to bind to dectin-1 can easily be determined with the methods as disclosed herein, especially in the example section. In case of doubt, a “dectin-1 binding β-glucan” is a β-glucan which binds to the soluble murine Fc-dectin-1a receptor with an IC50 value lower than 10 mg/ml, as determined by a competitive ELISA, e.g. as disclosed in the examples.

Dectin-1 binding β-glucans according to the present invention (such as linear β-(1,6)-glucans) are advantageous compared to other glucans, e.g. DC-SIGN P-glucans (such as β-(1,2)-glucans), because with such dectin-1 binding glucans a broader range of DCs may be addressed (immature, mature, myeloid, plasmacytoid; in addition: APCs) which significantly increases the potential to elicit an effective immune response in vivo compared to non-dectin-1 binding glucans (immature DCs, myeloid DCs) which limits applicability.

WO 2022/060487 A1 and WO 2022/060488 A1 disclose conjugates linking peptide immunogens to an immunostimulatory polymer molecule (e.g. β-(1,2) glucans). β-(1,2) glucans including cyclic variants have previously been implied as potential adjuvants (Martirosyan A et al., doi:10.1371/journal.ppat.1002983). They are a class of glucans which are predominantly binding to a specific PRR, DC-SIGN (Zhang H et al., doi:10.1093/glycob/cww041), specifically binding to N-linked high-mannose oligosaccharides and branched fucosylated structures. Importantly, β-1,2 glucans fail to bind to dectin 1 (Zhang H et al., doi:10.1093/glycob/cww041) thereby limiting their activity to DC-SIGN positive cells.

DC-SIGN (CD209) was the first SIGN molecule identified and found to be highly expressed on a restricted subset of DCs only, including immature (CD83-negative) DCs, as well as on specialized macrophages in the placenta and lung (Soilleux E J et al., doi: 10.1189/jlb.71.3.445). In the periphery, e.g. in skin or at mucosal sites, expression and hence the potential to be biologically active as receptor according to this invention is only detectable in subsets of immature DCs. Mature, plasmacytoid DCs and other APCs like epithelial DC-like Langerhans cells do not express DC-SIGN (Engering A, et al., doi:10.4049/jimmunol.168.5.2118) In contrast thereto, the target receptor of the β-glucan based immunogens as provided in the present invention is dectin-1. Dectin-1 is expressed on a variety of different DC types, including not only immature DCs, myeloid DCs but also plasmacytoid DCs, which express dectin-1 in both mRNA and protein levels as well as DC-like Langerhans cells in the skin (Patente et al., doi: 10.3389/fimmu.2018.03176; Joo et al., doi: 10.4049/jimmunol.1402276).

Hence, biological activity of DC-SIGN targeting polymers like β-(1,2) glucans is limited to specific DC target cell populations whereas dectin-1 targeting polymers as applied in this present invention can exert their function in a variety of different additional DC-types. Therefore, these novel conjugates can exert a significantly different and superior immune response as compared to other conjugates. The prior art disclosure therefore does not suggest the claimed subject matter disclosed by the present invention.

According to a specifically preferred embodiment, the conjugates of the present invention comprise a strong dectin-1 binding β-glucan, preferably a β-glucan which binds to the soluble murine Fc-dectin-1a receptor with an IC50 value lower than 10 mg/ml, more preferred with an IC50 value lower than 1 mg/ml, even more preferred with an IC50 value lower than 500 μg/ml, especially with an IC50 value lower than 200 μg/ml, as determined by a competitive ELISA, e.g. as disclosed in the examples. Specifically preferred are conjugates which bind to the soluble murine Fc-dectin-1a receptor with an IC50 value lower than 1 mg/ml, more preferred with an IC50 value lower than 500 μg/ml, even more preferred with an IC50 value lower than 200 μg/ml, especially with an IC50 value lower than 100 μg/ml, as determined by a competitive ELISA; and/or—a β-glucan which binds to the soluble human Fc-dectin-1 receptor with an IC50 value lower than 10 mg/ml, more preferred with an IC50 value lower than 1 mg/ml, even more preferred with an IC50 value lower than 500 μg/ml, especially with an IC50 value lower than 200 μg/ml, as determined by a competitive ELISA; and/or—wherein the conjugates bind to the soluble human Fc-dectin-1a receptor with an IC50 value lower than 1 mg/ml, more preferred with an IC50 value lower than 500 μg/ml, even more preferred with an IC50 value lower than 200 μg/ml, especially with an IC50 value lower than 100 μg/ml, as determined by a competitive ELISA, e.g. as disclosed in the examples.

Moreover, the conjugates according to the present invention also showed a proportionally highly increased ratio of antibodies reacting to target polypeptide than to carrier molecules as in non-CLEC, especially non-pustulan containing vaccines. This significantly increases the specific focus of the antibody immune response to the target rather than the carrier which then results in an increased efficacy and specificity of the response.

The CLEC conjugation according to the present invention, especially to pustulan, also leads to increased affinity maturation (AM) towards target proteins (AM is increased strongly, whereas KLH/CRM conjugates only show limited AM upon repeated immunization).

In the field of vaccines, suitable vaccines have been disclosed with only B-cell epitopes or only T-cell epitopes. There are specific circumstances where it is appropriate and preferred to have vaccines with exclusively T-cell epitopes or exclusively B-cell epitopes. However, most of the vaccines on the market contain both kinds of epitopes, i.e. T-cell epitopes and B-cell epitopes.

For example, vaccines containing only B-cell epitopes are in most cases not very effective, even though they do lead to a detectable antibody immune response. In most cases, however, this immune response is usually much less effective compared to a vaccine containing B- and T-cell epitopes. This is also in line with the examples given in the example section of the present invention wherein a lower level of response was detectable.

On the other hand, vaccines which only contain T-cell epitopes (e.g. in vaccines where a specific T-cell response would be the active component of the response), are specifically interesting for certain applications, especially for cancer, where cancer specific cytotoxic T lymphocyte and T-helper cell epitopes or only CTL epitopes are combined with the vaccine platform according to the present invention. In this case a T-cell epitope with the CLEC polysaccharide adjuvant according to the present invention is provided with the T-cell epitope only. This is specifically preferred e.g. in cases where somatic mutations in cancers affect protein coding genes which can give rise to potentially therapeutic neoepitopes. These neoepitopes can guide adoptive cell therapies and peptide- (and RNA-based) neoepitope vaccines to selectively target tumor cells using autologous patient cytotoxic T-cells. Using a vaccine with only T-cell epitopes may also be preferred with respect to specific autoimmune diseases. The treatment effect of the respective T-cell epitope only conjugate is associated with a reduction of effector T-cells and the development of regulatory T-cell (T_{reg}-cell) populations which leads to the dampening of the respective autoimmune disease (e.g.: multiple sclerosis or similar diseases).

Since most of the usual vaccine set-ups contain both, B-cell and T-cell epitopes, also the CLEC conjugates according to the present invention therefore preferably comprise both, individual B- and T-cell epitopes (at minimum: at least one B-cell epitope of alpha synuclein and at least one T-cell epitope) for a sustained B-cell immune response. However, a weak effect may demonstrate T-cell independent immunity if required.

The conjugates according to the present invention are therefore not limited with respect to possible vaccine antigens. Therefore, the alpha synuclein vaccines according to the present invention may additionally comprise further antigens to provide bitri-, tetra, penta-, hexa- (etc.), or multi-specific vaccines.

It is preferred that the vaccine antigens (i.e. B-cell and/or T-cell epitope polypeptides) have a length of 6 to 50 amino acid residues, preferably of 7 to 40 amino acid residues, especially of 8 to 30 amino acid residues.

A cross-linking of B-cell receptors is also possible using the vaccines according to the present invention. According to a specific embodiment, the conjugates according to the present invention are used for a T-cell independent immunization. T-cell independent responses are well known for polysaccharide vaccines. These vaccines/the polysaccharide produces an immune response by direct stimulation of B-cells, without the assistance of T-cells. The T-cell independent antibody response is short-lived. Antibody concentrations for pneumococcal capsule polysaccharides decline to baseline in typically 3-8 years, depending on serotype. Usually, additional doses cannot be used to enhance the vaccine response, as the polysaccharide vaccine does not constitute immunological memory. In children under two years of age, the polysaccharide vaccine is poorly immunogenic. Here the reason for direct stimulation could be that B-cells express a molecule called CR3 (complement receptor type 3). Macrophage-1 antigen or CR3 is a human cell surface receptor found on B- and T-lymphocytes, polymorphonuclear leukocytes (mostly neutrophils), NK cells, and mononuclear phagocytes like macrophages. CR3 also recognizes iC3b when bound to the surface of foreign cells and β-Glucan which means that direct uptake of the vaccine by B-cells via Pus-CR3 interaction could lead to the stimulation of the cells and the development of a low level TI immune response.

The adjuvants, conjugates and vaccines according to the present invention could fix complement and may be opsonized. Opsonized conjugates according to the present invention could have an increased B-cell activating ability which could lead to higher antibody titers and antibody affinity. This effect is known for C3d conjugates (Green et al., J. Virol. 77 (2003), 2046-2055) and is unexpectedly also useable in the course of the present invention.

Another unexpected advantage of the present invention is that the CLEC architecture of the present invention allows a modular design of the vaccine. For example, epitopes can be combined at will and the platform is independent from conventional carrier molecules. Although the major emphasis of the present invention is on peptide-only vaccines, it also works with independent coupling of proteins and peptides as well as with coupling of peptide-protein conjugates to the CLEC backbones according to the present invention, especially to pustulan. As shown in the example section with pustulan a significant superior immune response as compared to classical vaccines is obtained according to the present invention.

As already outlined above, the conjugates according to the present invention, if provided in a pharmaceutical preparation (e.g. as a vaccine intended to be administered to a (human) subject to elicit an immune response to a specific polypeptide epitope conjugated to the CLEC backbone, to which epitope the immune response should be elicited), can be administered without the need to use (by co-administration) a (further) adjuvant in this preparation. According to a preferred embodiment, the pharmaceutical formulation comprising the conjugate according to the present invention is free of adjuvants.

A specifically preferred class of CLEC polysaccharide adjuvants according to the present invention are β-glucans, especially pustulan. Another preferred CLEC polysaccharide adjuvant is mannan. In contrast to the present invention, pustulan has only been used in the prior art for anti-fungal vaccines (where pustulan was used as antigen and not as carrier as in the present invention). Pustulan is also displaying a different main chain as it only consists of β-(1,6)-linked sugar moieties.

Pustulan is a medium sized linear β-(1,6) glucan. Pustulan as well as synthetic forms of linear β-(1,6) glucan are different from all other glucans used as β-glucans usually consist of branched glucan chains (preferably β-(1,3) main chains with β-(1,6) side chains like yeast extracts, GPs, laminarin, schizophyllan, scleroglucan) or linear glucans only relying on β-(1,3) glucans like synthetic β-Glucan, curdlan, S. cerevisiae β-glucan (150 kDa) or linear β-(1,3:1,4) glucans like barley- and oat β-glucan as well as lichenan.

As shown for the first time with the present invention, the binding of glucan conjugates to the dectin-1 receptor in vitro is a surrogate for subsequent in vivo efficacy: low binding molecules can only exert low immune responses, medium binders are better whereas highly efficient binders induce highly efficient responses (oat/barley BG<lichenan<pustulan).

According to the present invention, the CLECs are coupled (e.g. by standard techniques) to individual alpha synuclein polypeptides to create small nanoparticles with low polydispersity (range of the hydrodynamic radius (HDR): 5-15 nm) which are not crosslinked and do not aggregate to form larger particulates similar to conventional CLEC vaccines such as glucan particles (2-4 μm) or β-glucan particles as disclosed in the literature, usually characterized by a size range of >100 nm (typical range (diameter; 150-500 nm, e.g. Wang et al. (2019) provide particles with a diameter of 160 nm (assessed by DLS) and a size of ca. 150 nm as assessed by TEM; Jin et al. (2018) provide β-glucan particles (nanoparticles of aminated β-glucan-ovalbumin) with 180-215 nm size (as assessed by DLS and SEM, respectively). These conjugates preferably comprise at least one T-cell epitope, especially a promiscuous, a linear or a carrier peptide T-cell epitope (e.g. from CRM197 or KLH).

By definition, the DLS measured hydrodynamic radius is the radius of a hypothetical hard sphere that diffuses with the same speed as the particle under examination. The radius is calculated from the diffusion coefficient assuming globular shape of your molecule/particle and a given viscosity of a buffer. The HDR is also called Stokes radius and is calculated from the diffusion coefficient using the Stokes—Einstein equation (see https://en.wikipedia.org/wiki/Stokes radius).

Preferred size ranges of the nanoparticles according to the present invention may be those typically provided in the prior art, i.e. with a size of 1 to 5000 nm, preferably of 1 to 200 nm, especially of 2 to 160 nm, determined as hydrodynamic radius (HDR) by dynamic light scattering (DLS). According to a preferred embodiment of the present invention, the particle size is smaller, e.g. from 1 to 50 nm, preferably from 1 to 25 nm, especially from 2 to 15 nm, determined as HDR by DLS. These preferred particles are therefore smaller, including the peptide only conjugates (about 5 nm average HDR) and CRM-pustulan conjugates (about 10-15 nm average HDR). Accordingly, preferred particles according to the present invention are smaller than 100 nm, this would separate us from Wang et al.

Accordingly, the present invention also relates to a vaccine product designed for vaccinating an individual against a specific alpha synuclein antigen, wherein the product comprises a compound comprising a β-glucan or mannan as a C-type lectin (CLEC) polysaccharide adjuvant covalently coupled to the specific antigen.

Preferably, the vaccine product according to the present invention comprises a conjugate as disclosed herein or obtainable or obtained by a method according to the present invention.

According to a preferred embodiment, the vaccine product according to the present invention comprises an alpha synuclein antigen comprising at least one alpha synuclein B-cell epitope and at least one T-cell epitope, preferably wherein the antigen is a polypeptide comprising one or more B-cell and T-cell epitopes.

According to a preferred embodiment, the covalently coupled antigen and CLEC polysaccharide adjuvant in the vaccine product according to the present invention are present as particles with a size of 1 to 5000 nm, preferably of 1 to 200 nm, especially of 2 to 160 nm, determined as hydrodynamic radius (HDR) by dynamic light scattering (DLS). As used herein, all particle sizes are median particle sizes, wherein the median is the value separating the half of the particles with a higher size from the half of the particles with lower size. It is the determined particle size from which half of the particles are smaller and half are larger.

According to a preferred embodiment, the covalently coupled antigen and CLEC polysaccharide adjuvant in the vaccine product according to the present invention are present as particles with a size of 1 to 50 nm, preferably of 1 to 25 nm, especially of 2 to 15 nm, determined as HDR by DLS.

Preferably, the covalently coupled antigen and CLEC polysaccharide adjuvant in the vaccine product according to the present invention are present as particles with a size smaller than 100 nm, 50 nm, preferably smaller than 70 nm, especially smaller than 50 nm, determined as HDR by DLS.

The vaccine products according to the present invention show a high storage stability. Virtually no aggregation takes place upon storage as liquid or frozen material (storage temperature: −80° C., −20° C., 2-8° C. or at room temperature over extended time periods, at least 3 months) as can be determined that the particle size does not significantly (i.e. more than 10%) increase over storage.

The extremely high efficacy of such small particles produced by using the medium molecular weight component pustulan according to the present invention is surprising: For example, according to Adams et al. (J Pharmacol Exp Ther. 2008 April; 325(1):115-23) the best dectin-1 substrates are linear β(1,3) glucan phosphate (ca. 150 kda) and branched glucans (containing a β(1,3) main chain and β-(1,6) side chains) like Scleroglucans or glucans from C. albicans or laminarin. In addition, the data of Adams et al., Palma et al. (J Biol Chem. 281(9) (2006) 5771-5779) and Willment et al. (J Biol Chem. 276(47) (2001), 43818-23) imply that dectin-1 does not or only weakly interact with pustulan, nor that it interacts with non-glucan carbohydrate polymers, such as mannan. In fact, various references report pustulan as being less effective in dectin-1 binding. In general, however, linear 1,3 and branched (1,3 main chain and 1,6 side branch) are the most effective dectin-1 binders; Adams et al. (2008) show that murine recombinant dectin-1 only recognized and interacted with polymers that contained a β(1,3)-linked glucose backbone. Dectin-1 did not interact with a glucan that was exclusively composed of a β-(1,6)-glucose backbone (such as pustulan), nor did it interact with non-glucan carbohydrate polymers, such as mannan.

In contrast to these findings, it was shown in the course of the present invention that pustulan based conjugates are able to strongly bind to dectin-1 and to elicit cellular responses in vitro.

According to a preferred embodiment of the present invention, a β-(1,6)-glucan is used. Usually large particulates are reported in the prior art to be more effective in activating PRRs than small (“soluble”) monomeric formulations, so particles containing large glucans are superior (and therefore preferred) and small, soluble glucans can be used to block activation of DCs thereby interfering with the intended effect. It is well accepted that particulate β-glucans, such as the widely used yeast cell-wall fraction zymosan, bind to and activate dectin-1 thereby inducing cellular responses. In contrast, the interaction of soluble β-glucans with dectin-1 is subject to debate. The general consensus, though, is that soluble β-glucans, such as the small, branched glucan laminarin (β-(1,3) and β-(1,6) side chains), bind to dectin-1 but are unable to initiate signaling and induce cellular responses in the DCs (Willment et al., J Biol Chem. 276(47) (2001), 43818-23, Goodridge et al. Nature. 2011, 472(7344): 471-475.).

According to the present invention, it could be shown that conjugates using high mol. weight glucans (10× the size of pustulan; e.g.: oat/barley 229 kDa/lichenan 245 kDa) perform less effective than pustulan particles (20 kDa). Korotchenko et al. show that OVA/Lam conjugates have a ca 10 nm diameter, bind dectin-1 and induce DC activation in vitro but are branched glucans, not skin specific and regarding the effect in vivo not superior compared to OVA applied into the skin or OVA/alum applied s.c. Wang et al. provide β-glucan particles with >100 nm size (average size: 160 nm). Jin et al. (2018) show aminated β-glucan-ovalbumin nanoparticles with 180-215 nm size.

According to the present invention, it was shown that pustulan-based particles are strong dectin-1 binders, activate DCs in vitro (changes in surface marker expression) and elicit a very strong immune response, superior to a) other routes and b) comparable to KLH/CRM conjugate vaccines (usually also much bigger particles) and C) larger glucans and also mannan. This is true for Pep+Padre+pustulan (size of 5 nm) and for Pep+CRM197+pustulan conjugates (size of 11 nm).

For optimal immune responses, the degree of activation of the CLEC, esp. pustulan, and the peptide/sugar ratio resulting from this degree of activation is decisive. Activation of the respective CLEC is achieved by mild periodate oxidation. Thus, the degree of oxidation is determined based on adding the periodate solution at a defined molar ratio: i.e. periodate:sugar subunit; 100%=1 Mol periodate per Mol sugar monomers.

According to a preferred embodiment, the conjugates according to the present invention comprise a CLEC activated with a ratio of periodate to β-glucan or mannan (monomer) moiety of 1/5 (i.e. 20% activation) to 2,6/1 (i.e. 260% activation), preferably of 60% to 140%, especially 70% to 100%.

The optimal range of oxidation degree (which will be directly proportional to the number of epitope polypeptides in the final conjugate) between a low/middle oxidation degree and a high degree of oxidation can be defined as the reactivity with Schiff's fuchsin-reagent similar to that of an equal amount of the given carbohydrate (e.g. pustulan) oxidized with periodate at a molar ratio (sugar monomer: periodate) of 0.2-0.6 (low/middle), 0.6-1.4 (optimal range) and 1.4-2.6 (high), respectively.

Preferred glucan to peptide ratios, especially pustulan to peptide ratios, are ranging from 10 to 1 (w/w) to 0.1 to 1 (w/w), preferably 8 to 1 (w/w) to 2 to 1 (w/w), especially 4 to 1 (w/w)), with the proviso if the conjugate comprises a carrier protein, the preferred ratio of β-glucan or mannan to B-cell-epitope-carrier polypeptide is from 50:1 (w/w), to 0.1:1 (w/w), especially 10:1 to 0.1:1; i.e. 24 to 1 molar ratio of sugar monomer to peptide), which are lower than effective vaccines reported elsewhere (e.g. Liang et al., Bromuro et al.).

The degree of oxidation and the amount of reactive aldehydes available for coupling of the sugar is determined using state of the art methods like: 1) gravimetric measurement allowing for determination of the total mass of the sample; 2) the anthrone method (according to Laurentin et al. 2003)—for concentration determination of intact, non-oxidized sugars in the sample; in this case glucans are dehydrated with concentrated H₂SO₄ to form Furfural, which condenses with anthrone (0.2% in H₂SO₄) to form a green color complex which can be measured colorimetrically at 620 nm) or 3) Schiff's assay: Oxidation status of carbohydrates used for conjugation is assessed using Schiff's fuchsin-sulfite reagent. Briefly, fuchsin dye is decolorized by Sulphur dioxide. Reaction with aliphatic aldehydes (on Glucan) restores the purple color of fuchsin, which can then be measured at 570-600 nm. Resulting color reaction is proportional to the oxidation degree (the amount of aldehyde groups) of the carbohydrate. Other suitable analytical methods are possible as well. Peptide ratios can be assessed using suitable methods including UV analysis (205 nm/280 nm) and amino acid analysis (aa hydrolysis, derivatization and RP-HPLC analysis).

The conjugates according to the present invention can be used to induce target-specific immune responses while inducing no or only very limited CLEC- or carrier protein-specific antibody responses. The conjugates according to the present invention can further be used for the induction of alpha synuclein specific immune responses while inducing no or only very limited CLEC- or carrier-protein specific antibody responses. As also shown in the example section below, the present invention also enables an improvement and focusing to the alpha synuclein-specific immune response because it triggers the immune response away from reactions to the carrier protein or the CLEC (as e.g. in conventional peptide-carrier conjugates or non-conjugated comparative set-ups, especially also applying non-oxidised CLECs, such as pustulan).

Unless indicated to the contrary, “peptides” as used herein refer to shorter polypeptide chains (of 2 to 50 amino acid residues) whereas “proteins” refer to longer polypeptide chains (of more than 50 amino acid residues). Both are referred to as “polypeptides”. The B-cell and/or T-cell epitope polypeptides conjugated to the CLECs according to the present invention comprise besides the polypeptides with the naturally used amino acid residues of normal gene expression and protein translation also all other forms of such polypeptide-based B-cell and/or T-cell epitopes, especially naturally or artificially modified forms thereof, such as glycopolypeptides und all other post-translationally modified forms thereof (e.g. the pyro-Glu forms of Aβ as disclosed in the examples). Moreover, the CLECs according to the present invention are specifically suitable for presenting conformational epitopes, for example conformational epitopes which are part of larger native polypeptides, mimotopes, cyclic polypeptides or surface-bound constructs.

According to a preferred embodiment, the conjugate according to the present invention comprises a CLEC polysaccharide backbone and a B-cell epitope. A “B-cell epitope” is the part of the alpha synuclein antigen that immunoglobulin or antibodies bind. B-cell epitopes can be divided into two groups: conformational or linear. There are two main methods of epitope mapping: either structural or functional studies. Methods for structurally mapping epitopes include X-ray crystallography, nuclear magnetic resonance, and electron microscopy. Methods for functionally mapping epitopes often use binding assays such as western blot, dot blot, and/or ELISA to determine antibody binding. Competition methods determine if two monoclonal antibodies (mAbs) can bind to an antigen at the same time or compete with each other to bind at the same site. Another technique involves high-throughput mutagenesis, an epitope mapping strategy developed to improve rapid mapping of conformational epitopes on structurally complex proteins. Mutagenesis uses randomly/site-directed mutations at individual residues to map epitopes. B-cell epitope mapping can be used for the development of antibody therapeutics, peptide-based vaccines, and immunodiagnostic tools (Sanchez-Trincado et al., J. Immunol. Res. 2017-2680160). For many antigens, B-cell epitopes are known and may be used in the present CLEC platform.

According to a specifically preferred embodiment, the conjugate according to the present invention comprises a CLEC polysaccharide backbone and a promiscuous T-cell epitope and/or a MHCII epitope which are known to work with several/al MHC alleles of a given species as well as in other species.

According to another aspect, the present invention also relates to the use of the present CLEC technology to improve known T-cell epitopes. Accordingly, the present invention also encompasses a β-glucan or mannan for use as a C-type lectin (CLEC) polysaccharide adjuvant for T-cell epitope polypeptides, wherein the β-glucan or mannan is covalently conjugated to the T-cell epitope polypeptide to form a conjugate of the β-glucan or mannan and the T-cell epitope polypeptide.

A single T-cell epitope which binds to more than one HLA allele is referred to as “promiscuous T-cell epitope”. Preferred promiscuous T-cell epitopes bind to 5 or more, preferably 10 or more, especially 15 or more, HLA alleles. Promiscuous T-cell epitopes are suitable for different species and most importantly for several MHC/HLA haplotypes (referring to both, MHCI and MHCII epitopes which are known to work with several/all MHC alleles) of a given species as well as in other species. For example, the MHCII epitope PADRE (=nonnatural pan DR epitope (PADRE)), as referred to in the example section, works in several human MHC alleles and in mouse (C57/Bl6, although it is less effective in Balb/c). According to a preferred embodiment, the conjugate of the present invention comprises a T-cell epitope, preferably a T-cell epitope comprising the amino acid sequence AKFVAAWTLKAAA (“PADRE (polypeptide)”) or a PADRE (polypeptide) variant.

Preferred PADRE polypeptides or PADRE polypeptide variants include a linker (as also preferred for other polypeptides epitopes used herein), such as a cysteine residue or a linker comprising a cysteine reside (“—C” or “C—”; specifically for maleimide coupling), an NRRA, NRRA-C or NRRA-NH—NH2 linker. Preferred PADRE polypeptide variants include the variants disclosed in the prior art (e.g. in Alexander et al., Immunity 1 (1994), 751-761; U.S. Pat. No. 9,249,187 B2, or), preferably a shortened variant without the C-terminal A residue (AKFVAAWTLKAA), variants wherein the first residue alanine is replaced by an aliphatic amino acid residue (e.g. glycine, valine, isoleucine and leucine), variants wherein the third residue phenylalanine is replaced with L-cyclohexylalanine, variants wherein the thirteenth (last) amino acid residue alanine is replaced by an aliphatic amino acid residue (e.g. glycine, valine, isoleucine and leucine), variants comprising aminocaproic acid, preferably coupled to the C-terminus of the PADRE variant, or variants with the amino acid sequence AX₁FVAAX₂TLX₃AX₄A, wherein X₁ is selected from the group consisting of W, F, Y, H, D, E, N, Q, I, and K; X₂ is selected from the group consisting of F, N, Y, and W, X₃ is selected from the group consisting of H and K, and X₄ is selected from the group consisting of A, D, and E (with the proviso that the oligopeptide sequence is not AKFVAAWTLKAAA; U.S. Pat. No. 9,249,187 B2); especially wherein the T-cell epitope is selected from AKFVAAWTLKAAANRRA-(NH—NH2), AKFVAAWTLKAAAN-C, AKFVAAWTLKAAAC, AKFVAAWTLKAAANRRA-C, aKXVAAWTLKAAaZC, aKXVAAWTLKAAaZCNRRA (SeqID7, 8, 87, 88, 89, 90, 91, 92), aKXVAAWTLKAAa, aKXVAAWTLKAAaNRRA, aA(X)AAAKTAAAAa, aA(X)AAATLKAAa, aA(X)VAAATLKAAa, aA(X) IAAATLKAAa, aK(X)VAAWTLKAAa, and aKFVAAWTLKAAa (sequences 760.5, 760.57, 906.09, 906.11, 965.10, 1024.03 from Alexander et al., 1994), wherein X is L-cyclohexylalanine, Z is aminocaproic acid and a is an aliphatic amino acid residue selected from alanine, glycine, valine, isoleucine and leucine.

T-cell epitopes are presented on the surface of an antigen-presenting cell, where they are bound to major histocompatibility complex (MHC) molecules. In humans, professional antigen-presenting cells are specialized to present MHC class II peptides, whereas most nucleated somatic cells present MHC class I peptides. T-cell epitopes presented by MHC class I molecules are typically peptides between 8 and 11 amino acids in length, whereas MHC class II molecules present longer peptides, 13-17 amino acids in length; non-classical MHC molecules also present non-peptidic epitopes such as glycolipids. MHC class I and II epitopes can be reliably predicted by computational means alone, although not all in-silico T-cell epitope prediction algorithms are equivalent in their accuracy. There are two main methods of predicting peptide-MHC binding: data-driven and structure-based. Structure based methods model the peptide-MHC structure and require great computational power. Data-driven methods have higher predictive performance than structure-based methods. Data-driven methods predict peptide-MHC binding based on peptide sequences that bind MHC molecules (Sanchez-Trincado et al., 2017). By identifying T-cell epitopes, scientists can track, phenotype, and stimulate T-cells. For many antigens, including alpha synuclein, T-cell epitopes are known and may be used in the present CLEC platform.

Interestingly, recent breakthrough studies have demonstrated that alpha-synuclein-specific T-cells are increased in PD patients, probably in association with risk haplotypes of HLA, and suggest an autoimmune involvement of T-cells in PD (Sulzer et al., Nature 2017; 546:656-661 and Lindestamn Arlehamn et al., Nat Commun. 1875; 2020:11). A causal role of alpha-synuclein reactive T-cells was recently reinforced also by an animal model study (Williams et al., Brain. 2021; 144:2047-2059). The occurrence of alpha-synuclein-reactive T-cells was increased years before motor onset in a case study and their frequency was highest around and shortly after motor onset in a larger cross-sectional cohort of PD patients (Lindestam Arlehamn et al.). After motor onset, the T-cell response to alpha-synuclein declined with increasing disease duration. Thus, anti aSyn T-cell responses are highest before or shortly after diagnosis of motor PD and wane thereafter (i.e. maximum activity detectable less than 10 years after diagnosis; and Hoehn and Yahr (H+Y) stages 1 and 2 are preferred) (Lindestamn Arlehamn et al. 2020).

Accordingly, there are commonly known T-cell epitopes contained within the sequence of human alpha synuclein. Examples are provided in Benner et al. (PLoS ONE 3(1): e1376.60), Sulzer et al., (2017) and Lindestam Arlehamn et al. (2020).

Benner et al (Benner et al., (2008) PLoS ONE 3(1): e1376.) use a 60 aa long nitrated (at Y-residues) polypeptide comprising the C-terminal part of aSyn emulsified in an equal volume of CFA containing 1 mg/ml Mycobacterium tuberculosis as immunogen in a PD model and disclose the alpha synuclein T-cell epitope aa71-86 (VTGVTAVAQKTVEGAGNIAAATGFVK).

Sulzer et al. (Nature 2017; 546:656-661) identified two T-cell antigenic regions at the N-terminal and C-terminal regions in alpha synuclein in human PD patients. The first region is located near the N terminus, composed of the MHCII epitopes aa31-45 (GKTKEGVLYVGSKTK) and aa32-46 (KTKEGVLYVGSKTKE) also containing the 9mer polypeptide aa37-45 (VLYVGSKTK) as potential MHCI class epitope. The second antigenic region disclosed by Sulzer et al. is near the C terminus (aa116-140) and required phosphorylation of amino acid residue S129. The three phosphorylated aaS129 epitopes aa116-130 (MPVDPDNEAYEMPSE), aa121-135 (DNEAYEMPSEEGYQD), and aa126-140 (EMPSEEGYQDYEPEA) produced markedly higher responses in PD patients than in healthy controls. The authors also demonstrate that the naturally occurring immune responses to alpha synuclein associated with PD have both MHC class I and II restricted components.

In addition, Lindestam Arlehamn et al. (Nat Commun. 1875; 2020:11) also disclose the alpha synuclein peptide aa61-75 (EQVTNVGGAVVTGVT) as T-cell epitope (MHCII) in PD patients.

Accordingly, preferred T-cell epitopes according to the present invention include the alpha synuclein polypeptides GKTKEGVLYVGSKTK (aa31-45), KTKEGVLYVGSKTKE (aa32-46), EQVTNVGGAVVTGVT (aa61-75), VTGVTAVAQKTVEGAGNIAAATGFVK (aa71-86), DPDNEAYEMPSE (aa116-130), DNEAYEMPSEEGYQD (aa121-135), and EMPSEEGYQDYEPEA (aa126-140).

The regulatory T-cells (“Treg cells” or “Tregs”) are a subpopulation of T-cells that modulate the immune system, maintain tolerance to self-antigens, and prevent autoimmune disease. Treg cells are immunosuppressive and generally suppress or downregulate induction and proliferation of effector T-cells. Tregs produced by a normal thymus are termed “natural”. The selection of natural Tregs occurs on radio-resistant haematopoietically-derived MHC class II-expressing cells in the medulla or Hassal's corpuscles in the thymus. The process of Treg selection is determined by the affinity of interaction with the self-peptide MHC complex. Selection to become a Treg is a “Goldilocks” process—i.e. not too high, not too low, but just right, a T-cell that receives very strong signals will undergo apoptotic death; a cell that receives a weak signal will survive and be selected to become an effector cell. If a T-cell receives an intermediate signal, then it will become a regulatory cell. Due to the stochastic nature of the process of T-cell activation, all T-cell populations with a given TCR will end up with a mixture of Teff and Treg—the relative proportions determined by the affinities of the T-cell for the self-peptide-MHC. Treg formed by differentiation of naïve T-cells outside the thymus, i.e. the periphery, or in cell culture are called “adaptive” or “induced” (i.e. iTregs).

Natural Treg are characterised as expressing both the CD4 T-cell co-receptor and CD25, which is a component of the IL-2 receptor. Treg are thus CD4+ CD25+. Expression of the nuclear transcription factor Forkhead box P3 (FoxP3) is the defining property which determines natural Treg development and function. Tregs suppress activation, proliferation and cytokine production of CD4+ T-cells and CD8+ T-cells, and are thought to suppress B-cells and dendritic cells thereby dampening autoimmune reactions.

Along these lines several studies indicate that Treg number and function is reduced in PD patients. E.g.: Hutter Saunders et al. (J Neuroimmune Pharmacol (2012) 7:927-938) and Chen et al. (MOLECULAR MEDICINE REPORTS 12: 6105-6111, 2015) show impaired abilities of regulatory T-cells (Treg) from PD patients to suppress effector T-cell function and that the proportion of Th1 and Th17 cells was increased, while that of Th2 and Treg cells was decreased. Thome et al. (npj Parkinson's Disease (2021) 7:41) showed that declining PD Treg function correlates with increasing proinflammatory T-cell activation which can directly result in the subsequent increase in pro-inflammatory signaling by other immune cell populations. Treg suppression of T-cell proliferation significantly correlated with peripheral pro-inflammatory immune cell phenotypes. The suppressive capacity of PD Tregs on T-effector cells (e.g.: CD4+) proliferation decreased with increasing PD disease burden using the H&Y disease scale with highest activity at stages H+Y 1 and 2. Importantly, Lindestam Arlehamn et al. (2020) showed that anti aSyn T-cell responses are highest before or shortly after diagnosis of motor PD and wane thereafter (i.e. maximum activity detectable less than 10 years after diagnosis; and Hoehn and Yahr (H+Y) stages 1 and 2 are preferred) (Lindestamn Arlehamn et al., 2020).

Thus, the combination of the vaccines according to the present invention with
1) vaccines containing an alpha synuclein specific Treg epitope (e.g. a CD4 epitope like those disclosed by Brenner et al, Sulzer et al. and Lindestam Arlehamn et al. (aa31-45 (GKTKEGVLYVGSKTK), aa32-46 (KTKEGVLYVGSKTKE), aa61-75 (EQVTNVGGAVVTGVT), aa71-86 (VTGVTAVAQKTVEGAGNIAAATGFVK), aa116-130 (MPVDPDNEAYEMPSE), aa121-135 (DNEAYEMPSEEGYQD), and aa126-140 (EMPSEEGYQDYEPEA)); and/or
2) with Treg inducing agents like rapamycin, low-dose IL-2, TNF receptor 2 (TNFR2) agonist, anti-CD20 antibodies (e.g.: rituximab), prednisolone, inosine pranobex, glatiramer acetate, sodium butyrate
   is preferred at early stages of the disease (i.e. less than 10 years after diagnosis; and Hoehn and Yahr stages 1 and 2 are preferred) to augment waning/reduced Treg number and activity and thereby reduce autoimmune reactivity of aSyn specific T-effector cells and dampen autoimmune responses in PD patients.

More to this, Tregs are found to be decreased and/or dysfunctional in a number of diseases, especially chronic degenerative or autoimmune diseases such as (active) systemic lupus erythematosus (SLE, aSLE), type 1 diabetes (T1D), autoimmune diabetes (AID), multiple sclerosis (MS), amyotrophic lateral sclerosis (ALS), and Alzheimer's disease (AD) among other degenerative diseases (ALS: Beers et al., JCI Insight 2, e89530 (2017); AD: Faridar et al., Brain Commun. 2, fcaa112 (2020); ALS: Beers et al., JAMA Neurol. 75, 656-658 (2018); MS: Haas et al., Eur. J. Immunol. 35, 3343-3352 (2005); T1D: Lindley et al., Diabetes 54, 92-99 (2005): AID: Putnam et al., J. Autoimmun. 24, 55-62 (2005); autoimmune diseases: Ryba-Stanislawowska et al., Expert Rev. Clin. Immunol. 15, 777-789 (2019); aSLE: Valencia et al., J. Immunol. 178, 2579-2588 (2007); MS: Viglietta et al., J. Exp. Med. 199, 971-979 (2004); sLE: Zhang et al., Clin. Exp. Immunol. 153, 182-187 (2008); AD+MS: Ciccocioppo et al., Sci. Rep. 9, 8788 (2019)).

It is therefore also preferred to provide T-cell epitopes suitable as Treg epitopes or Treg inducing agents in diseases with reduced or dysfunctional Treg populations as a combination with the vaccines according to present invention to augment waning/reduced Treg number and activity and thereby reduce autoimmune reactivity of disease specific T-effector cells and dampen autoimmune responses in patients. Whereas suitable Treg epitopes are defined as self MHC epitopes (MHCII type) which are characterized by the ability to induce intermediate signals during T-cell selection processes.

According to a preferred embodiment, the conjugate according to the present invention comprises a polypeptide comprising or consisting of the amino acid sequences SeqID7, 8, 22-29, 87-131, GKTKEGVLYVGSKTK, KTKEGVLYVGSKTKE, EQVTNVGGAVVTGVT, VTGVTAVAQKTVEGAGNIAAATGFVK, MPVDPDNEAYEMPSE), DNEAYEMPSEEGYQD, EMPSEEGYQDYEPEA, or combinations thereof.

Preferred T-cell epitopes are therefore:

| | | |
|---|---|---|
| SeqID7 | AKFVAAWTLKAAANRRA-(NH-NH2) | PADRE |
| SeqID8 | AKFVAAWTLKAAAN-C | PADRE |
| SeqID22 | AKFVAAWTLKAAA-(NH-NH2) | PADRE - original |
| SeqID23 | KAAAVKAAFWTAL-NRRA-(NH-NH2) | alternative synthetic peptide |
| SeqID24 | DSETADNLEKTVAALSILPGHGC- | Toxin Diphtheria (TV-TT ex- |
| | (NH-NH2) | change) |
| SeqID25 | DSETADNLEKTVAALSILPGHGCNRRA- | Toxin Diphtheria (TV-TT ex- |
| | (NH-NH2) | change) |
| SeqID26 | ISITEIKGVIVHRIETILF-(NH-NH2) | MvF5 Th (UBITh ® 1) |
| SeqID27 | ISITEIKGVIVHRIETILENRRA- | MvF5 Th (UBITh ® 1) |
| | (NH-NH2) | |
| SeqID28 | ISQAVHAAHAEINEAGR-(NH-NH2) | chicken Ova (323-339) |
| SeqID29 | ISQAVHAAHAEINEAGRNRRA- | chicken Ova (323-339) |
| | (NH-NH2) | |
| SeqID87 | AKFVAAWTLKAAA-C | Padre (original) for Malei- |
| | | midecoupling |
| SeqID88 | AKFVAAWTLKAAANRRA-C | Tridem' Padre (original) for |
| | | Maleimidecoupling |
| SeqID89 | aKXVAAWTLKAAaZC | PADRE, alternative Aas |
| SeqID90 | aKXVAAWTLKAAaZCNRRA | PADRE, alternative Aas |
| SeqID91 | aKXVAAWTLKAAa | PADRE, alternative Aas |
| SeqID92 | aKXVAAWTLKAAaNRRA | PADRE, alternative Aas |
| SeqID93 | DSETADNLEKTTAALSILPG | Diphteria |
| SeqID94 | DSETADNLEKTTAALSILPGNRRA | Diphteria |
| SeqID95 | LSEIKGVIVHRLEGV | MvF |
| SeqID96 | LSEIKGVIVHRLEGVNRRA | MvF |
| SeqID97 | KLLSLIKGVIVHRLEGVE | MvF |
| SeqID98 | KLLSLIKGVIVHRLEGVENRRA | MvF |
| SeqID99 | VSIDKFRIFCKANPK | P23 - TT |
| SeqID100 | LKFIIKRYTPNNEIDS | P32 - TT |
| SeqID101 | IREDNNTLKLDRCNN | P21 - TT |
| SeqID102 | FNNFTVSFWLRVPKVSASHLE | P30 - TT |
| SeqID103 | QYIKANSKFIGITE | P2 - TT |
| SeqID104 | LEYIPEITLPVIAALSIAES | TT |
| SeqID105 | LINSTKIYSYFPSVISKVNQ | TT |
| SeqID106 | NYSLDKIIVDYNLQSKITLP | TT |
| SeqID107 | PHHTALRQAILCWGELMTLA | HBV nuclear capsid |
| SeqID108 | FELLTRILTIPOSLD | HBV surface AG |
| SeqID109 | YSGPLKAEIAQRLEDV | MT Influenza matrix epitope |
| SeqID110 | FELLTRILTIPQSL | HBsAg |
| SeqID111 | GAYARCPNGTRALTVAELRGNAEL | Bordetella pertussis |
| SeqID112 | ALNIWDRFDVFCTLGATTGYLKGNS | Cholera Toxin |
| SeqID113 | QYIKANSKFIGITEL | Clostridium tetani TT1 |
| SeqID114 | FNNFTVSFWLRVPKVSASHLE | Clostridium tetani TT2 |
| SeqID115 | KFIIKRYTPNNEIDSE | Clostridium tetani TT3 |
| SeqID116 | VSIDKFRIFCKALNPK | Clostridium tetani TT4 |
| SeqID117 | WVRDIIDDETNESSQKT | Clostridium tetani 2 |
| SeqID118 | AGLTLSLLVICSYLFISRG | EBV BHRF1 |
| SeqID119 | PGPLRESIVCYFMVELQTHI | EBV EBNA-1 |
| SeqID120 | VPGLYSPCRAFFNKEELL | EBV CP |
| SeqID121 | TGHGARTSTEPTTDY | EBV GP340 |
| SeqID122 | KELKRQYEKKLRQ | EBV BPLF1 |
| SeqID123 | TVFYNIPPMPL | EBV EBNA-2 |
| SeqID124 | DKREMWMACIKELH | HCMV IE1 |
| SeqID125 | FVFTLTVPSER | Influenza MP1 |
| SeqID126 | PKYVKQNTLKLAT | Influenza Hemagluttinin |
| SeqID127 | EKKIAKMEKASSVENV | Malaria CS: T3 epitope |
| SeqID128 | FELLTRILTI | Hepatitis B surface antigen |
| SeqID129 | DQSIGDLIAEAMDKVGNEG | Heat Shock Protein 65 |
| SeqID130 | QVHFQPLPPAVVKL | bacille CalmetteGuerin |
| SeqID131 | KQIINMWQEVGKAMYA | HIV gp120 |

wherein X is L-cyclohexylalanine, Z is aminocaproic acid and a is an aliphatic amino acid selected from alanine, glycine, valine, isoleucine and leucine.

According to another preferred embodiment, the conjugate according to the present invention comprises a B-cell epitope of alpha synuclein and a T-cell epitope, preferably a pan-specific/promiscuous T-cell epitope, independently coupled to the CLEC polysaccharide backbone according to the present invention, especially to pustulan.

According to another preferred embodiment, the conjugate according to the present invention comprises an alpha synuclein B-cell epitope coupled to a “classic” carrier protein, such as CRM197, wherein this construct is further coupled to a CLEC carrier according to the present invention, especially to pustulan.

For example, in a first step, CRM conjugate formation may be performed by activation of CRM via GMBS or sulfo-GMBS etc.; then the maleimide-groups of the activated CRM are reacted with SH groups of the peptide (cysteine). CRM conjugates are then treated with DTT to reduce disulphide bonds and generate SH-groups on cysteins. Subsequently, a one pot reaction mixing reduced CRM-conjugate with BMPH (N-β-maleimide-propionic acid hydrazide) and activated pustulan (oxidised) may be done to create the CLEC-based vaccine. The mechanism in the one pot reaction may be (with respect to pustulan) that oxidised pustulan is reacted with BMPH (has the hydrazide residues) and to form a BMPH-hydrazone. The reduced CRM conjugate is then reacting via SH groups on CRM-conjugate with the maleimide of the BMPH activated pustulan.

According to another preferred embodiment, the conjugates according to the present invention comprise a “classical” carrier protein, such as CRM197, containing multiple T-cell epitopes. The conjugate according to the present invention also comprises a B-cell epitope covalently coupled to the polysaccharide moiety. In this embodiment, both polypeptides (B-cell epitope and carrier molecule) are coupled independently to a CLEC carrier according to the present invention, especially to pustulan.

According to another preferred embodiment, the conjugates according to the present invention also comprise a “classical” carrier protein, such as CRM197, containing multiple T-cell epitopes. The conjugate according to the present invention also comprises a B-cell epitope covalently coupled to the “classical” carrier protein. The peptide-carrier conjugate according to the present invention is also covalently coupled to the polysaccharide moiety. In this embodiment, both polypeptides (B-cell epitope and carrier molecule) are coupled as one conjugate to a CLEC carrier according to the present invention, especially to pustulan. The carrier protein then represents a link between the β-glucan or mannan and the B-cell and/or T-cell epitope polypeptide(s) in the conjugate according to the present invention. The covalent conjugation between the β-glucan or mannan and the B-cell and/or T-cell epitope polypeptides is then made by the carrier protein (as a functional linking moiety). Preferred conjugates according to the present invention may comprise a B-cell epitope coupled to CRM197, wherein this construct is further coupled to a CLEC polymer according to the present invention especially to a β-glucan wherein the β-glucan is pustulan, lichenan, laminarin, curdlan, β-glucan peptide (BGP), schizophyllan, scleroglucan, whole glucan particles (WGP), zymosan, or lentinan, preferably pustulan, laminarin, lichenan, lentinan, schizophyllan, or scleroglucan, especially pustulan.

According to the present invention, it was shown that novel B-cell epitope-CRM197 conjugates coupled to pustulan are strong dectin-1 binders and elicit a very strong immune response, superior to conventional CRM conjugate vaccines.

According to the present invention it was shown that CLEC conjugation to novel B-cell epitope+CRM197 conjugates, especially creating B-cell epitope+CRM197+β-glucan, more preferably B-cell epitope+CRM197+linear β-(1,6)-Glucan or B-cell epitope+CRM197+pustulan conjugates is indispensable for induction of the superior immunogenicity described for various peptide+CRM197+CLEC, especially peptide+CRM197+β-glucan, more preferably peptide+CRM197+linear β-(1,6)-glucan or peptide+CRM197+linear pustulan conjugates as compared to conventional CRM conjugate vaccines with or without adjuvantation by mixing with β-glucan/pustulan.

It is therefore a preferred embodiment to provide B-cell epitope+CRM197 conjugates covalently linked to β-glucan, more preferably peptide+CRM197+linear β-(1,6)-glucan or peptide+CRM197+linear pustulan conjugates.

Accordingly, the present invention also relates to the improvement and/or optimisation of carrier proteins by covalently coupling the carrier protein (already containing one or more T-cell antigens (as part of its polypeptide sequence, optionally in post-translationally-modified form)) to the CLEC polysaccharide adjuvant according to the present invention, i.e. the β-glucan or mannan, preferably to pustulan, lichenan, laminarin, curdlan, β-glucan peptide (BGP), schizophyllan, scleroglucan, whole glucan particles (WGP), zymosan, or lentinan. The present invention therefore relates to a β-glucan or mannan for use as a C-type lectin (CLEC) polysaccharide adjuvant for B-cell and/or T-cell epitope alpha synuclein polypeptides, wherein the β-glucan or mannan is covalently conjugated to the B-cell and/or T-cell epitope polypeptide to form a conjugate of the β-glucan or mannan and the B-cell and/or T-cell epitope polypeptide, wherein a carrier protein is covalently coupled to the β-glucan or mannan.

This improvement/optimization leads to a significant reduction or elimination of the B-cell response to the CLEC and/or to the carrier protein and/or an enhancement (or at least preservation) of the T-cell response to the T-cell epitopes of the carrier protein. This enables a reduction or elimination of an antibody-response to the CLEC and/or the carrier (which then only delivers a T-cell response) and a specific enhancement of the antibody-response to the actual target polypeptide which is conjugated to the carrier and the CLEC.

Accordingly, a specifically preferred embodiment of the present invention is a conjugate consisting of or comprising
(a) a β-glucan or a mannan
(b) at least a B-cell or T-cell epitope polypeptide, and
(c) a carrier protein,
   wherein the three components (a), (b) and (c) are covalently conjugated with each other.

This combination of these three components can be provided in any orientation or sequence, i.e. in the sequence (a)-(b)-(c), (a)-(c)-(b) or (b)-(a)-(c), wherein (b) and/or (c) can be covalently conjugated either from the N-terminus to the C-terminus or from the C-Terminus to the N-terminus or conjugated via a functional group within the polypeptide (e.g. via a functional group in a lysine, arginine, aspartic acid, glutamic acid, asparagine, glutamine, serine, threonine, tyrosine, tryptophan or histidine residue, especially via the s-ammonium group of a lysine residue). Of course, the β-glucan or mannan can be coupled to one or more of each of the components (b) and (c), preferably by the methods disclosed herein. Preferably, these components are conjugated by linkers, especially by linkers between all at least three components. Preferred linkers are disclosed herein, such as a cysteine residue or a linker comprising a cysteine or glycine residue, a linker resulting from hydrazide-mediated coupling, from coupling via heterobifunctional linkers, such as BMPH, MPBH, EMCH or KMUH, from imidazole mediated coupling, from reductive amination, from carbodiimide coupling a —NH—NH₂ linker; an NRRA, NRRA-C or NRRA-NH—NH₂ linker, peptidic linkers, such as bi-, tri-, tetra- (or longer)-meric peptide groups, such as CG or CG. In the case of established carrier proteins, especially CRM, CRM197 and KLH, a preferred sequence of the at least three components is (a)-(c)(b), i.e. wherein the β-glucan or mannan and the least one B-cell or one T-cell epitope polypeptide is coupled to the carrier protein.

According to another preferred embodiment, the conjugates according to the present invention comprise a T-cell epitope and are free of B-cell epitopes, wherein the conjugate preferably comprises more than one T-cell epitope, especially two, three, four or five T-cell epitopes. This construct is specifically suitable for cancer vaccines. This construct is also specifically suitable for self-antigens, especially autoimmune disease associated self-antigens. The treatment effect of the respective conjugate is associated with a reduction of effector T-cells and the development of regulatory T-cell (T_{reg}-cell) populations which leads to the dampening of the respective disease, e. g. autoimmune disease or allergic disorders, for example as shown for multiple sclerosis. Notably, these T reg cells execute strong bystander immunosuppression and thus improve disease induced by cognate and noncognate autoantigens.

Preferred CLECs to be used as polysaccharide backbones according to the present invention are pustulan or other β-(1,6) glucans (including also synthetic forms of such glucans); others to be used: mannan, β-glucan family members, esp. linear β-(1,3) (S. cerevisiae β-glucan (e.g.: 150 kDa), curdlan) or branched β-(1,3) and β-(1,6) containing glucans, e.g.: laminarin (4.5-7 kDa), scleroglucan, schizophyllan, more preferably linear glucans, (e.g.: β-(1,3): S. cerevisiae β-glucan (150 kd), curdlan (75-80 kDa or bigger), β-(1,3)+β-(1,4) lichenan (22-250 kDa) β-(1,6) pustulan (20 kDa). Preferred CLECs according to the present invention are therefore mannan and β-glucans, including linear and branched β-glucans characterized by presence of β-(1,3)-, β-(1,3)+β-(1,4)- and β-(1,6) main chains as well as with attached side chains with β-(1,6) residues, more preferred linear β-glucans containing β-(1,3), β-(1,3)+β-(1,4) and β-(1,6) chains, more preferred linear β-(1,6) β-glucans, especially pustulan, fragments or synthetic variants thereof consisting of multimeric β-(1,6)-glucan saccharides (e.g. 4-mer, 5-mer, 6mer, 8-mer, 10-mer, 12-mer, 15-mer, 17-mer or 25mer).

Preferably, the minimum length of the CLECs according to the present invention is a 6-mer, because with smaller polysaccharides oxidation reactions as performed with the present invention are problematic (eventually other coupling mechanisms can be used for such smaller forms and/or terminally linking with addition of reactive forms). CLECs with 6 or more monomer units (i.e. 6-mers and larger-mers) show good dectin binding. Usually, the longer the CLEC, the better the dectin binding. The degree of polymerization (i.e. the amount of single glucose molecules within one glucan entity, DP) of 20-25 (i.e. DP20-25) definitely ascertains good binding and in vivo efficacy (e.g. laminarin is a typical example with a DP of 20-30).

Molecular weight of synthetic CLECs may also be smaller, Accordingly, e.g. as low as 1-2 kDa, whereas preferred molecular weight ranges of glucans and fragments thereof may be from 1 to 250 kDa (e.g. laminarin, lichenan, S. cerevisiae β-glucan, pustulan, curdlan and barley glucans, etc.), preferably from 4.5 to 80 kDa (e.g. laminarin, pustulan, curdlan, low molecular weight lichenan, etc.), especially 4.5 to 30 kDa (e.g. laminarin, pustulan, low MW lichenan, etc.).

Mannans are polysaccharides that are linear polymers of the sugar mannose. Plant mannans have β-(1,4) linkages. They are a form of storage polysaccharide. Mannan cell wall polysaccharide found in yeasts have an α-(1,6) linked backbone and α-(1,2) and α-(1,3) linked branches. It is serologically similar to structures found on mammalian glycoproteins.

In order to produce the conjugates according to the present invention, the CLEC, especially pustulan, must be activated (e.g. by using mild periodate mediated oxidation) and the degree of oxidation is important for the immune response. As already disclosed above, practical oxidation ranges are—specifically for pustulan—from about 20 to 260% oxidation. In many cases, the optimal oxidation range is between a low/middle oxidation (i.e. 20-60% oxidation) and a high degree of oxidation (i.e. 140-260% oxidation), i.e. in the range of 60-140% oxidation. Optimization for other CLECs may easily be adapted by a person skilled in the art, e.g. for lichenan more than 200% is necessary to gain a similar amount of aldehyde groups.

Accordingly, the ranges may alternatively also be defined as the reactivity with Schiff's fuchsin reagent which—for the example of pustulan—can be defined as follows: a low/middle oxidation degree at a molar ratio (sugar monomer:periodate) of 0.2-0.6, an optimal range of 0.6-1.4, and a high degree of oxidation of 1.4-2.6, respectively.

In any way, the degree of oxidation should be defined to meet the optimal range for each specific CLEC. Preferably, a linear β-glucan, more preferred a β-(1,6β-glucan, especially pustulan, pustulan fragments or synthetic variants thereof consisting of multimeric β-(1,6)-glucan saccharides (e.g. 4-mer, 5-mer, 6mer, 8-mer, 10-mer, 12-mer, 15-mer, 17-mer or 25-mer) is activated by mild periodate oxidation resulting in cleavage of vicinal OH groups and thus generation of reactive aldehydes. Mild periodate oxidation refers to the use of sodium periodate (NaIO₄), a well-known mild agent for effectively oxidizing vicinal diols in carbohydrate sugars to yield reactive aldehyde groups. The carbon-carbon bond is cleaved between adjacent hydroxyl groups. By altering the amount of periodate used, aldehydes can be stoichiometrically introduced into a smaller or larger number of sugar moieties of a given polysaccharide.

Other exemplary methods for activation of carbohydrates are well known in the art and include cyanylation of hydroxyls (e.g.: by use of organic cyanylating reagents, like 1-cyano-4-(dimethylamino)-pyridinium tetrafluoroborate (CDAP) or N-cyanotriethylammonium tetrafluoroborate (CTEA), reductive amination of carbohydrates or activation and coupling using Carboxylic acid-reactive chemical groups like Carbodiimides.

Activated carbohydrates are then reacted with the polypeptides to be coupled to the activated CLEC and allowed to form a conjugate of the CLEC with the B-cell or a T-cell epitope polypeptide.

Accordingly, the present invention also relates to a method for producing the conjugates according to the present invention, wherein the β-glucan or mannan is activated by oxidation and wherein the activated β-glucan or mannan is contacted with the B-cell and/or the T-cell epitope polypeptide, thereby obtaining a conjugate of the β-glucan or mannan with the B-cell and/or the T-cell epitope polypeptide.

Preferably, the β-glucan or mannan is obtained by periodate oxidation at vicinal hydroxyl groups, as reductive amination, or as cyanylation of hydroxyl groups.

According to a preferred embodiment, the β-glucan or mannan is oxidized to an oxidation degree defined as the reactivity with Schiff's fuchsin-reagent corresponding to an oxidation degree of an equal amount of pustulan oxidized with periodate at a molar ratio of 0.2-2.6 preferably of 0.6-1.4. especially 0.7-1.

Preferably, the conjugate is produced by hydrazone based coupling for conjugating hydrazides to carbonyls (aldehyde) or coupling by using hetero-bifunctional, maleimide-and-hydrazide linkers (e.g.: BMPH (N-β-maleimidopropionic acid hydrazide, MPBH (4-[4-N-maleimidophenyl]butyric acid hydrazide), EMCH (N-[ε-Maleimidocaproic acid) hydrazide) or KMUH (N-[κ-maleimidoundecanoic acid]hydrazide) for conjugating sulfhydryls (e.g.: cysteines) to carbonyls (aldehyde).

The polypeptides to be coupled to the CLECs according to the present invention are or comprise at least one B-cell or at least one T-cell epitope. Preferably, the polypeptide coupled to the CLECs contain a single B- or T-cell epitope (even in the embodiment when more than one kind of polypeptide is coupled to the CLEC polysaccharide backbone). As also shown in the example section, preferred lengths of the alpha synuclein polypeptides are from 5 to 29 amino acid residues, preferably from 5 to 25 amino acid residues, more preferred from 7 to 20 amino acid residues, even more preferred from 7 to 15 amino acid residues, especially from 7 to 13 amino acid residues. In this connection it is important to note that these length ranges are drawn to the epitope sequences only but do not include linkers, including peptidic linkers, such as cysteine or glycine or bi-, tri-, tetra- (or longer)-meric peptide groups, such as CG or CG, or cleavage sites, such as the cathepsin cleavage site; or combinations thereof (e.g. -NRRAC). Illustrative examples of epitopes have been tested in the example section; it follows from these results that the platform according to the present invention is not limited to any specific polypeptide. Therefore, virtually all possible epitopes are eligible for the present invention, including those epitopes which are already known in the present field and especially those which have already been described to be integrable into a presentation platform (e.g. together with a “classical” carrier molecule or adjuvant).

Epitopes are specifically preferred, if they can be coupled to activated β-glucan based on state-of-the-art coupling methods including hydrazide-mediated coupling, coupling via heterobifunctional linkers (e.g.: BMPH, MPBH, EMCH, KMUH etc.), imidazole mediated coupling, reductive amination, carbodiimide coupling etc. (more to be added). Epitopes used comprise individual peptides, can be contained within peptides or proteins or can be presented as peptide-protein conjugates before coupling to CLECs.

Preferred coupling methods to be used to provide the conjugates according to the present invention are therefore hydrazide coupling or coupling using thioester formation (e.g. maleimide coupling using BMPH (N-β-maleimidopropionic acid hydrazide), MPBH, EMCH, KMUH, especially where pustulan is coupled to the BMPH via hydrazone formation and the polypeptide is coupled via thioester.

In this embodiment, it is preferred to provide the polypeptides with two preferred linkers, such as hydrazide polypeptides/epitopes for hydrazone coupling:

N-terminal coupling of peptide: H₂N—NH—CO—CH₂—CH₂—CO-Polypeptide-COOH; preferably in combination with succinic acid or alternative suitable linkers, e.g. other suitable dicarboxylic acids, especially also glutaric acid used as a spacer/linker; C-terminal coupling (which is the preferred coupling orientation according to the present invention): NH₂—Polypeptide-NH—NH₂.

Alternatively, non-modified alpha synuclein polypeptides/epitopes may be applied in the present invention, e.g. polypeptides containing an (extra) cysteine residue or an alternative source for SH groups at either C- or N-terminus for heterobifunctional linker mediated coupling (especially BMPH, MPBH, EMCH, KMUH): NH₂—Cys-Pep-COOH or NH₂—Pep-Cys-COOH.

Preferred B-cell polypeptides to be used according to the present invention are polypeptides with a length of 5 to 19 amino acid residues, preferably 6 to 18 amino acid residues, especially 7 to 15 amino acid residues. The B-cell epitopes are preferably short, linear polypeptides, glycopolypeptides, lipopolypeptides, other post-translationally modified polypeptides (e.g.: phosphorylated, acetylated, nitrated, containing pyroglutamate residues, glycosylated etc.), cyclic polypeptides, etc.

Preferred T-cell polypeptides to be used according to the present invention have a length of 8 to 30 amino acid residues, preferably of 13 to 29 amino acid residues, more preferably of 13 to 28 amino acid residues.

Preferred specificities of the T-cell epitopes to be used in the present invention are short linear peptides suitable or known to be suitable for presentation via MHC I and II (as known to the person skilled in the art), especially MHCII epitopes for CD4 effector T-cells and CD4 Treg cells, MHCI epitopes for cytotoxic T-cell (CD8+) and CD8 Treg cells, for example useful for cancer, autoimmune or infectious diseases) with known efficacy in humans or animals; short linear peptides suitable for presentation via MHC I and II (as known to the person skilled in the art) with a N- or C-terminal addition of a lysosomal protease cleavage site, specifically a Cathepsin protease family member specific site, more specifically a site for cysteine cathepsins like cathepsins B, C, F, H, K, L, O, S, V, X, and W, especially a cathepsin S- or L-, most preferred a Cathepsin L cleavage site fostering efficient endo/lysosomal release of peptides for MHC presentation, especially MHCII with known efficacy in humans or animals. Cathepsin cleavage sites in various proteins have been identified and are well known in the art. This includes disclosures of sequences or methods to identify such sequences: e.g.: Biniossek et al., J. Proteome Res. 2011, 10, 12, 5363-5373; Adams-Cioaba et al., Nature Comm. 2011, 2:197; Ferrall-Fairbanks PROTEIN SCIENCE 2018 VOL 27:714-724; Kleine-Weber et al., Scientific Reports (2018) 8:1659, https://en.wikipedia.org/wiki/Cathepsin_S and others. Specifically, the adaption of peptide sequences using artificial protease cleavage sites as shown in the present invention is based on the surprising effect of these sequence extensions in eliciting more efficient immune responses following dermal application of the CLEC vaccines according to the present invention when the antigens are coupled to CLECs. Vaccines are according to the present invention are taken up by DCs and peptide antigens are subsequently lysosomally processed and presented at MHCs.

Lysosomes are intracellular membrane-bound organelles characterized by an acidic interior and harbor a variety of hydrolytic enzymes including lipases, proteases and glycosidases that participate in cellular catabolism. Among the variety of enzymes that lysosomes harbor, cathepsins are a family of lysosomal proteases with a broad spectrum of functions. All cathepsins fall into three different protease families: serine proteases (cathepsins A and G), aspartic proteases (cathepsin D and E) and eleven cysteine cathepsins. In humans eleven cysteine cathepsins are known which also have structures similar to that of papain: cathepsins B, C (J, dipeptidyl peptidase I or DPPI), F, H, K (O2), L, O, S, V (L2), X (P,Y,Z) and W (lymphopain).

Cathepsins exhibit similarities in their cellular localization and biosynthesis with some differences in their expression pattern. Of all the lysosomal proteases, cathepsins L, B, and D are the most abundant with their lysosomal concentrations equivalent to 1 mM. Cathepsins B, H, L, C, X, V, and O are ubiquitously expressed while cathepsins K, S, E, and W show cell or tissue-specific expression. Cathepsin K is expressed in osteoclasts and in epithelial cells. Cathepsins S, E, and W are mainly expressed in immune cells.

Besides their main function in lysosomal protein recycling, cathepsins play significant roles in a variety of physiological processes. Cathepsin S is the major protease involved in MHC II Ag processing and presentation. Cathepsin S null mice show a marked variation in generation of MHC II-bound li fragments and presentation, due to the substantially diminished li degradation in professional APCs where cathepsin S is abundantly expressed. In addition, endocytosis targets exogenous material selectively to cathepsin S in human DCs. Enrichment of MHC II molecules within late endocytic structures has consistently been noted in splenic DCs of cathepsin S-deficient mice as well. Recent studies suggest that both cathepsin B and D are involved, but not essential for MHC II-mediated Ag presentation as well. Cathepsin L also plays a role in a wide variety of cellular processes including antigen processing, tumor invasion and metastasis, bone resorption, and turnover of intracellular and secreted proteins involved in growth regulation. Although commonly recognized as a lysosomal protease, cathepsin L is also secreted. This broad-spectrum protease is potent in degrading several extracellular proteins (laminins, fibronectin, collagens I and IV, elastin, and other structural proteins of basement membranes) as well as serum proteins and cytoplasmic and nuclear proteins.

As a novel means to augment T-cell epitope efficacy in a vaccine, especially a CLEC based vaccine, a N- or C-terminal addition of a lysosomal protease cleavage site is provided as a preferred embodiment of the present invention.

Such cleavage sites according to the present invention may be characterized as follows:

### Cathepsin L-Like Cleavage Sites:

The intended Cathepsin L like cleavage site is defined based on protease cleavage site sequences known by the man skilled in the art, specifically also those as disclosed in Biniossek et al. (J. Proteome Res. 2011, 10, 5363-5373) and Adams-Cioaba et al. (Nature Comm. 2011, 2:197). The orientation of the site can be N- or C-terminally, preferred C-terminally. The preferred consensus sequence for C-terminal a Cathepsin L site is consisting of the formula:

Xₙ—X₁—X₂—X₃—X₄—X₅—X₆—X₇—X₈
Xₙ: 3-27 amino acids from the immunogenic peptide
X₁: any amino acid
X₂: any amino acid
X₃: any amino acid
X₄: N/D/A/Q/S/R/G/L; preferred N/D, more preferred N
X₅: F/R/A/K/T/S/E; preferred F or R, more preferred R
X₆: F/R/A/K/V/S/Y; preferred F or R, more preferred R
X₇: any amino acid, preferred A/G/P/F, more preferred A
X₈: cysteine or Linker like NHNH₂
   Most preferred sequence: Xₙ-X₁X₂X₃NRRA-Linker

### Cathepsin S Like Cleavage Site:

The intended Cathepsin S cleavage site is based on protease cleavage site sequences known by the man skilled in the art, specifically also those as disclosed in Biniossek et al. (J. Proteome Res. 2011, 10, 5363-5373) and in https://en.wikipedia.org/wiki/Cathepsin_S and is characterized by the consensus sequence:

Xₙ—X₁—X₂—X₃—X₄—X₅—X₆—X₇—X₈

Where X is characterised by
Xₙ: 3-27 amino acids from the immunogenic peptide
X₁: any amino acid
X₂: any amino acid
X₃: any amino acid, preferred V,L,I,F,W,Y,H, more preferred V
X₄: any amino acid, preferred V,L,I,F,W,Y,H, more preferred V
X₅: K,R, E, D, Q, N, preferably K, R more preferably R
X₆: any amino acid
X₇: any amino acid, preferred A
X₈: preferred A
X₈: cysteine or linker like NHNH₂
   Most preferred sequence: Xₙ-X₁X₂VVRAA-Linker

T-cell epitopes contained within proteins where the proteins are suitable for coupling to CLECs including carrier proteins, especially toxic cross-reactive material of diphtheria toxin (CRM), especially CRM₁₉₇, KLH, diphtheria toxoid (DT), tetanus toxoid (TT), Haemophilus influenzae protein D (HipD), and the outer membrane protein complex of serogroup B meningococcus (OMPC), recombinant non-toxic form of Pseudomonas aeruginosa exotoxin A (rEPA), flagellin, Escherichia coli heat labile enterotoxin (LT), cholera toxin (CT), mutant toxins (e.g., LTK63 and LTR72), virus-like particles, albumin binding protein, bovine serum albumin, ovalbumin, a synthetic peptide dendrimer e.g. a Multiple antigenic peptide (MAP) or other commercially available carrier proteins, preferably CRM197 and KLH, most preferred CRM197, preferably wherein the ratio of carrier protein to β-glucan or mannan in the conjugate is from 1/0.1 to 1/50, preferably 1/0.1 to 1/40, more preferred from 1/0.1 to 1/20, especially from 1/0.1 to 1/10.

According to preferred embodiments of the present invention, the CLEC conjugates according to the present invention comprise (a) CLECs conjugated with individual B-cell epitopes of alpha synuclein and/or T-cell epitopes, including mixes of B- or T-cell epitopes, especially these epitope(s) coupled to pustulan; (b) CLECs conjugated with polypeptide-carrier protein conjugates, preferably polypeptide-KLH or polypeptide CRM197 conjugates coupled to pustulan, most preferably, polypeptide-CRM197 conjugates coupled to pustulan; (c) CLECs conjugated with individual B-cell epitopes of alpha synuclein and T-cell epitopes; coupled to CLECs, most preferably to pustulan; (d) CLECs coupled individually (“individually” here means that the polypeptide chains are not present as a fusion protein, tandem repeat polypeptide or peptide-protein conjugate but as independent entities; i.e. an independent B-cell epitope-containing polypeptide and an independent T-cell epitope containing polypeptide) with B-cell epitopes and T-cell epitopes which are contained within polypeptides or proteins, e.g. carrier proteins, self-proteins, foreign proteins from pathogens, allergens etc.; (e) CLECs coupled individually (“individually”, again has the same meaning as for (d)) with T-cell epitopes representing linear MHCI and MHCII epitopes or which are contained within proteins, e.g. carrier proteins or target proteins for example for the treatment of neoplastic disease, or autoimmune disease.

In view of these advantageous properties of the conjugates of the present invention, it follows that the conjugates and vaccines according to the present invention are specifically useable for an active anti-alpha synuclein vaccine for the treatment and prevention of synucleopathies.

According to a further aspect, the present invention also relates to a pharmaceutical composition comprising a conjugate or vaccine as defined above and a pharmaceutically acceptable carrier.

Preferably, the pharmaceutically acceptable carrier is a buffer, preferably a phosphate or TRIS based buffer.

According to a preferred embodiment of the present invention, the pharmaceutical composition is contained in a needle-based delivery system, preferably a syringe, a mini-needle system, a hollow needle system, a solid microneedle system, or a system comprising needle adaptors; an ampoule, needle-free injection systems, preferably a jet injector; a patch, a transdermal patch, a microstructured transdermal system, a microneedle array patch (MAP), preferably a solid MAP (S-MAP), coated MAP (C-MAP) or dissolving MAP (D-MAP); an electrophoresis system, a iontophoresis system, a laser-based system, especially an Erbium YAG laser system; or a gene gun system. The conjugates according to the present invention are not limited to any form of production, storage or delivery state. All traditional and typical forms are therefore adaptable to the present invention. Preferably, the compositions according to the present invention may contain the present conjugates or vaccines in contained as a solution or suspension, deep-frozen solution or suspension; lyophilizate, powder, or granulate.

The present invention is further illustrated by the following examples and figures, however without being restricted thereto.

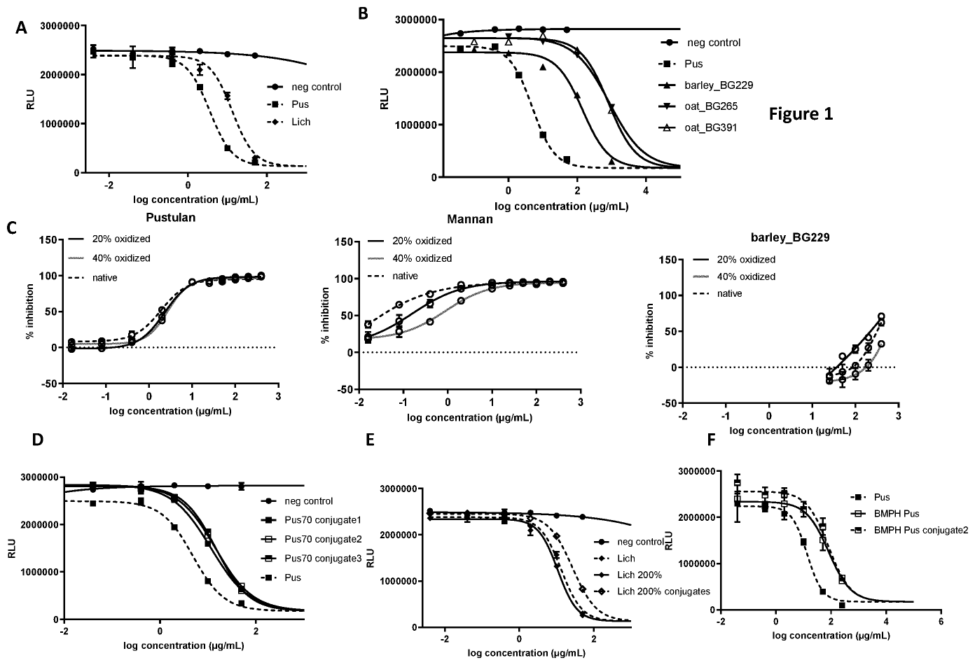

FIG. 1 shows: ConA and DC receptor (i.e. dectin-1) binding activity by CLEC-conjugates in vitro.

A) higher binding efficacy to dectin-1 is demonstrated for pustulan (Pus) when compared to lichenan (Lich) and B) beta-glucans from oat (oat_BG265, oat_BG391) and barley (Barley_BG229) showed limited binding efficacy in comparison to pustulan. C) different Glucan types (i.e., pustulan, mannan, and barley glucan (229 kd)) retain high or intermediate receptor binding activity following glucan oxidation as assessed by competitive binding assays. “20% and 40% oxidized” denotes the oxidation status of glucan moieties used for conjugation. % Inhibition indicates the inhibition of binding of soluble dectin-1 receptor (pustulan and barley_BG229) or ConA (mannan) to plate bound beta-glucan or mannan in the presence of the indicated concentrations of the tested CLEC. D) Pustulan-conjugates and E) lichenan-conjugates maintain approximately 50% of dectin-1 binding capacity compared to uncoupled beta-glucan, as assessed by competitive binding assay. F) Pustulan-conjugates produced via heterobifunctional linkers maintain high dectin-1 binding efficacy. Data are shown as relative light units (RLU) of a luminometric ELISA. Pus70 Conjugate 1-3 refers to three different CLEC peptide conjugates, respectively (SeqID2, SeqID10 and SeqID16). Pus 70% and Lich 200% refers to pustulan and lichenan with the respective oxidation status. BMPH Pus refers to activated pustulan. BMPH Conjugate 2 refers to CLEC-SeqID10 conjugate.

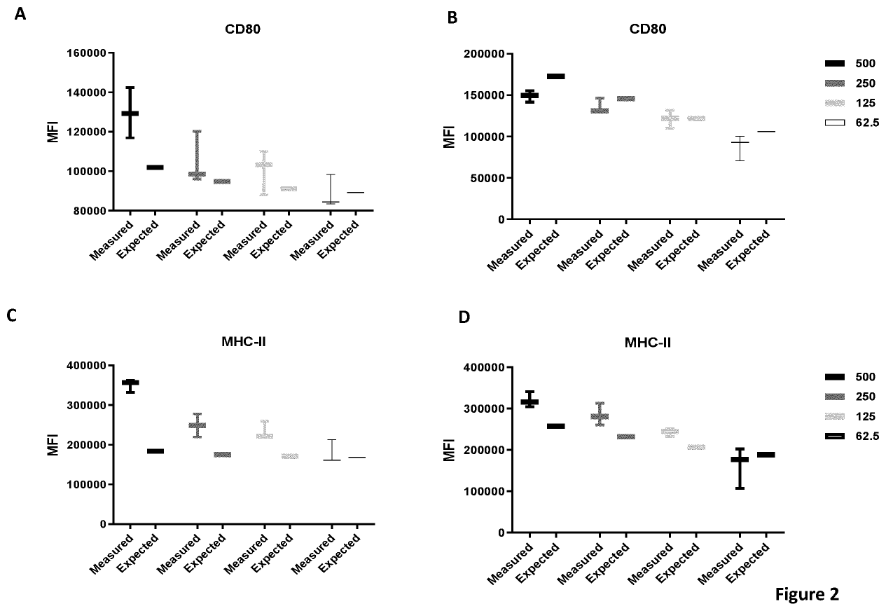

FIG. 2 shows: Flow cytometry analysis of dendritic cell activation by lipopolysaccharide (LPS) and different pustulan preparations.

Immature, bone marrow derived mouse dendritic cells (BMDCs) were generated in vitro, using granulocyte-macrophage colony-stimulating factor (GM-CSF). GM-CSF-BMDCs were stimulated with LPS (equivalent dose contained in oxidized pustulan and in pustulan-conjugate preparations), SeqID2+SeqID7+pustulan conjugates or oxidized pustulan only for 24 hours. Pustulan-conjugates and pustulan only were used in increasing doses starting at 62.5 μg/mL of the respective sugar (up to 500 μg/mL). DCs were identified based on CD11c/CD11b expression, and the surface expression of CD80 and major histocompatibility complex (MHC) class II by A) and C) SeqID2+SeqID7+pustulan conjugates or B) and D) oxidized pustulan only were measured by flow cytometry. The expression of activation markers was analyzed by CytExpert Software for DCs treated with pustulan-preparations (=measured) and DCs treated with equivalent amounts of LPS(=expected).

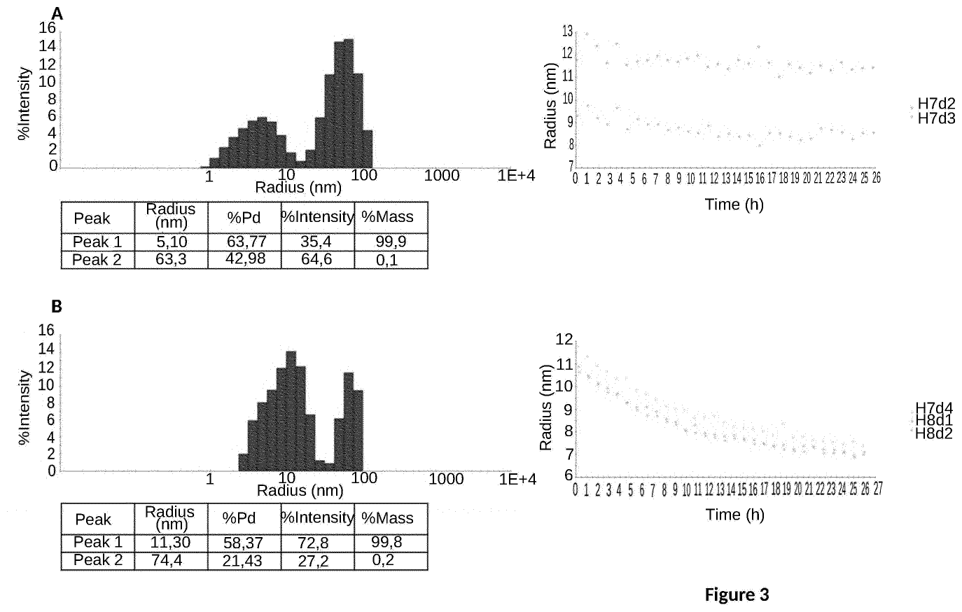

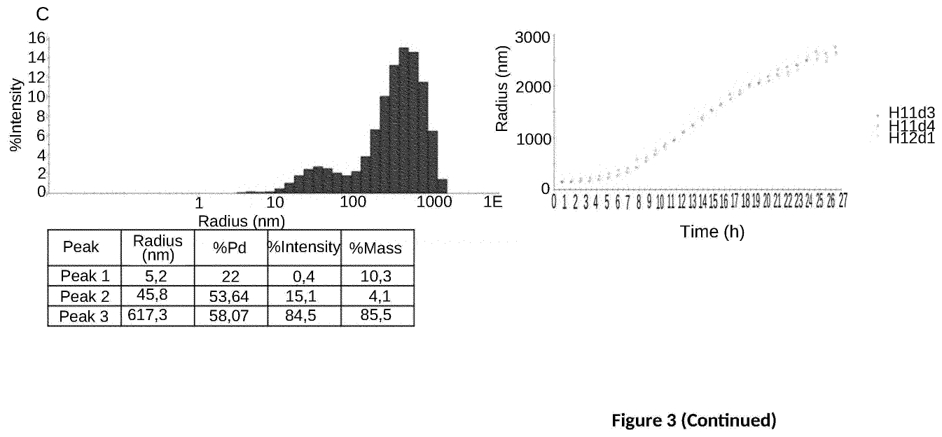

FIG. 3 shows: Particle size determination of CLEC-conjugates by dynamic light scattering (DLS).

Particle size has been determined by measuring the random changes in the intensity of light scattered from a suspension or solution by DLS. Regularisation analysis and the corresponding cumulant radius analysis over 24 hours, respectively, are shown for A) SeqID5+SeqID7+pustulan (80% oxidation status) conjugates, B) SeqID6+CRM+pustulan conjugates and C) non-modified pustulan.

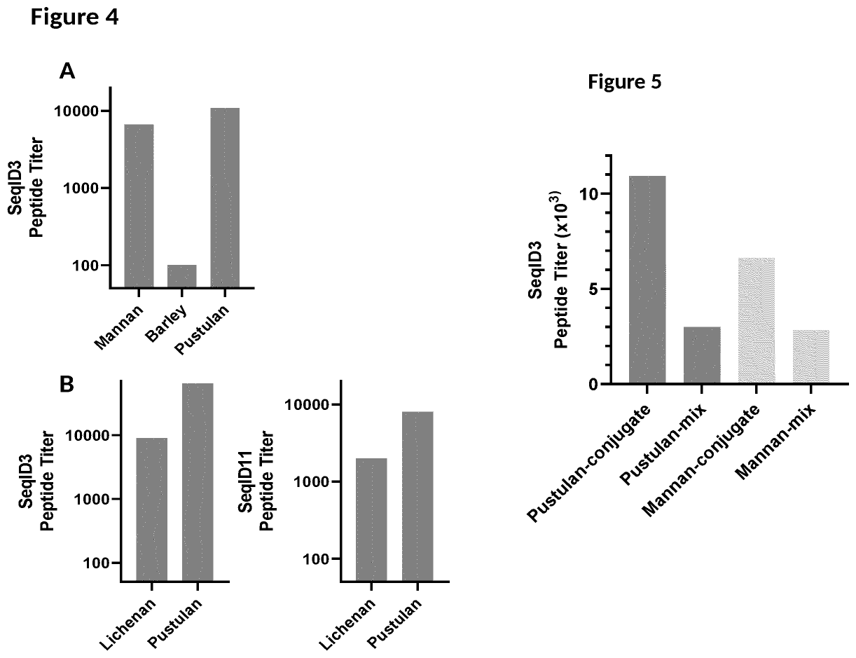

FIG. 4 shows: Comparison of immunogenicity of different CLEC based vaccines.

Female BALB/c mice, 8-12 weeks of age, received a total of 3 intradermal vaccinations applied at a 2-week interval. Blood samples have been collected at baseline and after each vaccination to inform on the kinetics of the ensuing immune response. Samples were taken 2 weeks after 3^{rd} application and analysed for A) anti-peptide response (SeqID3) of mannan-, barley- and pustulan-based vaccines (SeqID2+SeqID7+CLEC) and B) anti-peptide responses (Se-qID3 and SeqID11) of pustulan- and lichenan-based vaccine (Se-qID2+SeqID7+CLEC and SeqID10+SeqID7+CLEC).

FIG. 5 shows: Comparative analysis of the immunogenicity of peptide-pustulan conjugates and vaccines consisting of unconjugated peptides and CLECs.

Female BALB/c mice, 8-12 weeks of age received a total of 3 intradermal vaccinations applied at a 2-week interval. Blood samples have been collected at baseline and after each vaccination to inform on the kinetics of the ensuing immune response. Samples were taken 2 weeks after 3rd application and analysed for anti-peptide responses (SeqID3). Vaccines used: SeqID2+SeqID7+CLEC or mixes of unconjugated SeqID2, SeqID7 and CLEC.

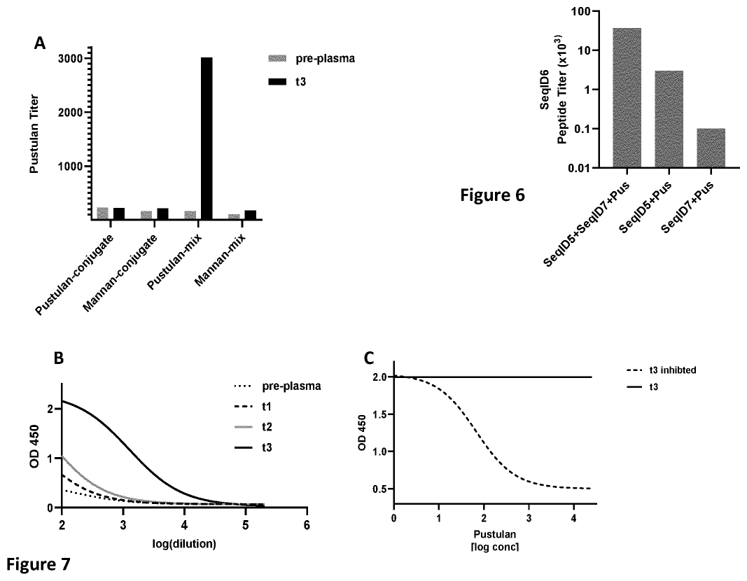

FIG. 6 shows: Comparative analysis of the immunogenicity of pustulan conjugates containing B- and T-cell epitopes to conjugates containing either the respective B-cell or T-cell epitope only.

Female BALB/c mice, 8-12 week of age received a total of 3 intradermal vaccinations applied at a 2-week interval. Blood samples have been collected at baseline and after each vaccination to inform on the kinetics of the ensuing immune response. Vaccines used: SeqID5+SeqID7+CLEC or SeqID5+CLEC, and SeqID7+CLEC. Samples were taken 2 weeks after 3rd application and analysed for anti-peptide responses (SeqID6).

FIG. 7 shows: Comparative analysis of anti-pustulan antibody responses in mice following repeated immunisation using pep-tide-pustulan conjugates or vaccines containing the respective non conjugated components

Female BALB/c mice, 8-12 weeks of age received a total of 3 intradermal vaccinations applied at a 2-week interval. Blood samples have been collected at baseline and after each vaccination to inform on the kinetics of the ensuing immune response. Pre-plasma and t1-t3 indicates immune responses detectable before (pre-plasma) or after the first (t1), 2nd (t2) or third (t3) immunization. Samples were taken 2 weeks after 3rd application and analysed for anti-pustulan responses. A) Analysis of the anti-pustulan response elicited by different vaccines. B) Kinetics of the immune response. C) Inhibition ELISA demonstrating the specificity of the ELISA system. Vaccines used: SeqID2+SeqID7+CLEC or mixes of unconjugated SeqID2, SeqID7 and CLEC

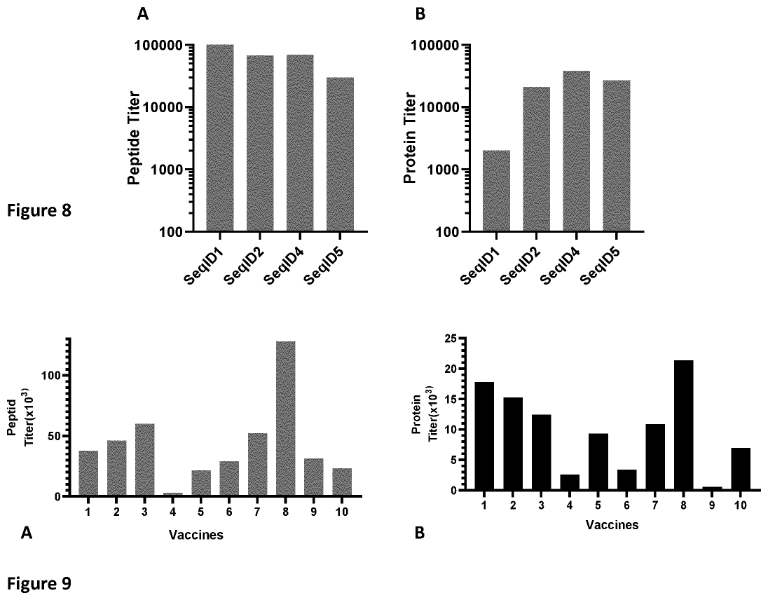

FIG. 8 shows: Comparative analysis of immune responses elicited by CLEC-based vaccines using differential peptide coupling orientation.

Female BALB/c mice, 8-12 weeks of age received a total of 3 intradermal vaccinations applied at a 2-week interval. Blood samples have been collected at baseline and after each vaccination to inform on the kinetics of the ensuing immune response. 4 different CLEC-based prototype vaccine candidates (two different peptides either coupled via their C- or N-terminus to pustulan) were tested. Samples were taken 2 weeks after 3rd application and analysed for A) anti-peptide and B) anti-aSyn protein responses. Vaccines used: SeqID1/2/4/5+SeqID7+CLEC

FIG. 9 shows: Comparative analysis of the immunogenicity of CLEC-based vaccines using different promiscuous T-helper cell epitopes.

Female BALB/c mice, 8-12 weeks of age received a total of 3 intradermal vaccinations applied at a 2-week interval. Blood samples have been collected at baseline and after each vaccination to inform on the kinetics of the ensuing immune response. Immune responses elicited by 9 different CLEC-based vaccines (Vaccine 1-9) containing the same B-cell epitope and different T-helper epitopes (i.e. SeqID7, SeqID22-29) were evaluated against the respective peptide-KLH conjugate (Vaccine 10), respectively. Samples were taken 2 weeks after 3rd application and analysed for A) anti-peptide and B) anti-aSyn protein responses.

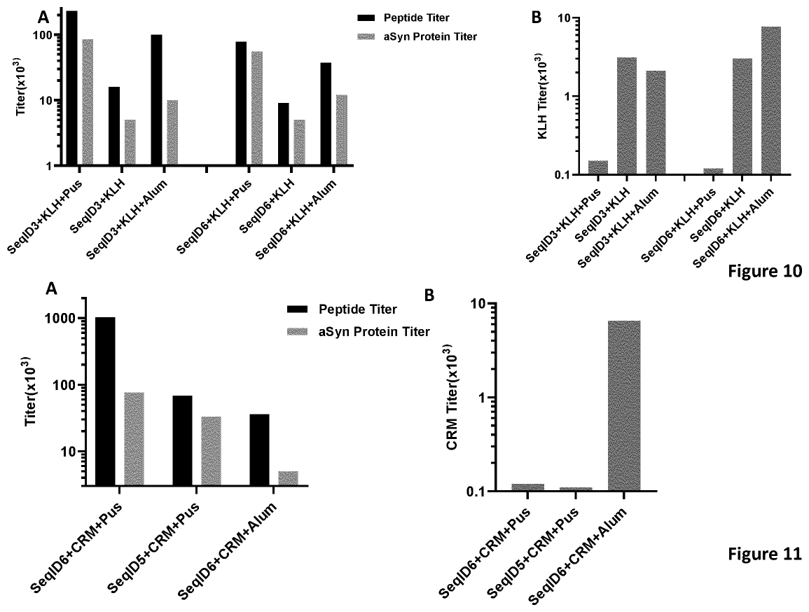

FIG. 10 shows: the Comparative analysis of the target- and carrier protein specific immunogenicity induced by CLEC-based- and conventional peptide-protein conjugate vaccines using the carrier protein KLH as source for T-helper cell epitopes.

Female BALB/c mice, 8-12 weeks of age received a total of 3 intradermal or sub cutaneous (s.c.) vaccinations applied at a 2-week interval. Blood samples have been collected at baseline and after each vaccination to inform on the kinetics of the ensuing immune response. Immune reactions elicited by 2 peptide-protein conjugate vaccines using KLH as source for T-helper epitopes in combination with CLEC modifications (SeqID3+KLH+pustulan and Se-qID6+KLH+pustulan, respectively) were evaluated against reactions induced by conventional peptide-KLH conjugates (i.e. SeqID3+KLH and SeqID6+KLH) either applied with Alum/Alhydrogel s.c. or without additional adjuvant i.d. Samples were taken 2 weeks after 3rd application and analysed for A) anti-peptide and anti-aSyn protein responses and B) anti-KLH responses by ELISA.

FIG. 11 shows: the Comparative analysis of the target- and carrier protein specific immunogenicity induced by CLEC-based- and conventional peptide-protein conjugate vaccines using the carrier protein CRM197 as source for T-helper cell epitopes

Female BALB/c mice, 8-12 weeks of age received a total of 3 intradermal or s.c. vaccinations applied at a 2-week interval. Blood samples have been collected at baseline and after each vaccination to inform on the kinetics of the ensuing immune response. 2 different CRM-based vaccine types have been used in this study. SeqID6+CRM+Pus represents a peptide-CRM conjugate which has been subsequently coupled to pustulan whereas SeqID5+CRM+Pus represents a conjugate where the peptide component and the carrier molecule have been coupled to the CLEC individually. Immune reactions induced by both types have been evaluated against the respective conventional peptide-CRM conjugate (i.e. SeqID6+CRM adjuvanted with Alum/Alhydrogel and applied s.c.). Samples were taken 2 weeks after 3rd application and analysed for A) anti-peptide and anti-aSyn protein responses and B) anti-CRM responses by ELISA.

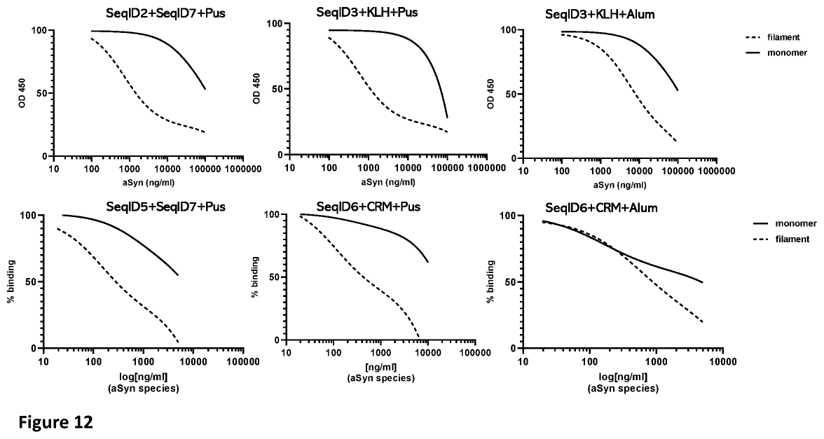

FIG. 12 shows: The comparative analysis of the selectivity of the immune responses elicited by CLEC based vaccines in vivo against two different aSyn forms.

Female BALB/c mice, 8-12 weeks of age received a total of 3 intradermal or s.c. vaccinations applied at a 2-week interval. CLEC based vaccine (SeqID2+SeqID7+Pus and SeqID5+SeqID7+Pus; applied i.d.) and alternative CLEC based vaccine (SeqID3+KLH+Pus and SeqID6+CRM+Pus; applied i.d.) were evaluated against conventional peptide-component vaccine (SeqID3+KLH+Alum and SeqID6+CRM+Alum, applied s.c.). Sample were taken 2 weeks after 3rd application and subjected to aSyn selectivity assay (inhibition ELISA). Black line: monomeric aSyn used for inhibition; dashed line: filamentous aSyn used for inhibition.

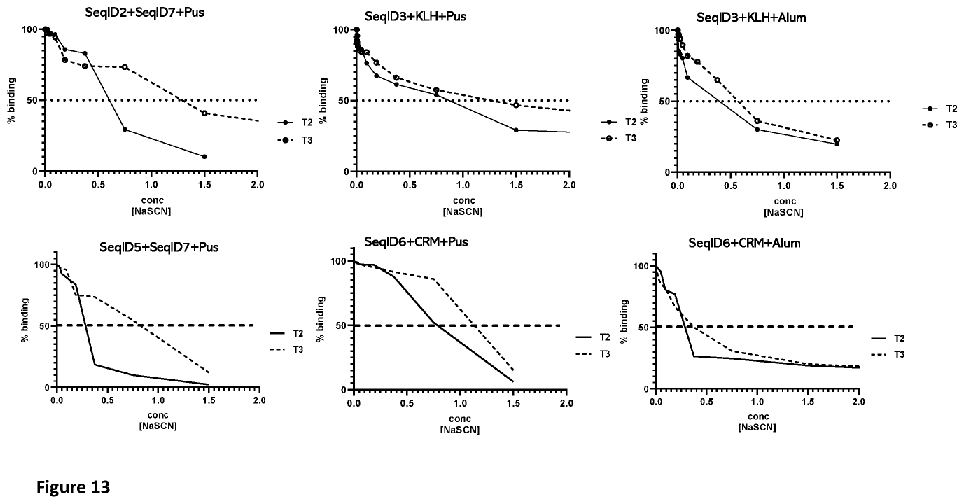

FIG. 13 shows: a comparative analysis of the avidity of immune responses elicited by CLEC based vaccines.

Female BALB/c mice, 8-12 weeks of age received a total of 3 intradermal or s.c. vaccinations applied at a 2-week interval. CLEC based vaccine (SeqID2+SeqID7+Pus and SeqID5+SeqID7+Pus, applied i.d.) and alternative CLEC based vaccine (SeqID3+KLH+Pus and SeqID6+CRM+Pus, applied i.d.) were evaluated against conventional peptide-component vaccine (SeqID3+KLH+Alum and SeqID6+CRM+Alum, applied s.c.). Samples were taken 2 weeks after the second (T2) or two weeks after the third immunization (T3) immunisation and antibody avidity to aSyn was assessed by ELISA based avidity assay.

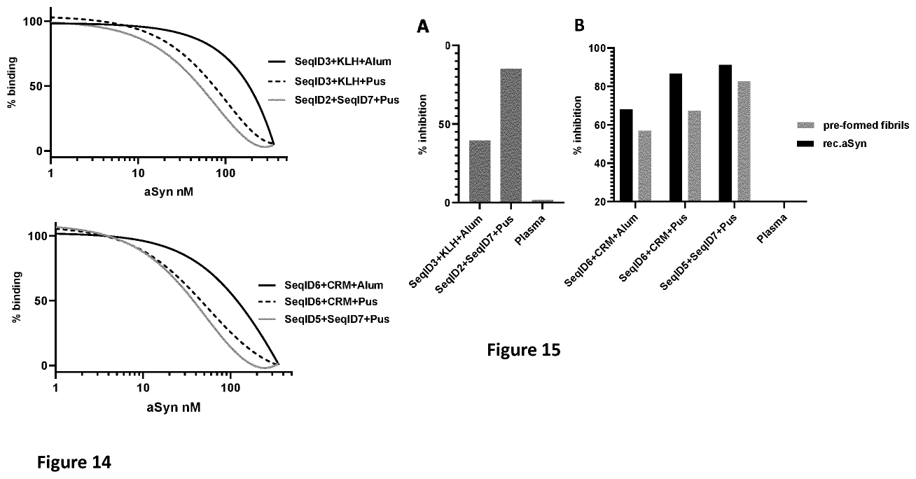

FIG. 14 shows: a comparative analysis of the affinity of immune responses elicited by CLEC based vaccines.

Female BALB/c mice, 8-12 weeks of age received a total of 3 intradermal or s.c. vaccinations applied at a 2-week interval. CLEC based vaccine (SeqID2+SeqID7+Pus and SeqID5+SeqID7+Pus, applied i.d.) and alternative CLEC based vaccine (SeqID3+KLH+Pus and SeqID6+CRM+Pus, applied i.d.) were evaluated against conventional peptide-component vaccine (SeqID3+KLH+Alum and SeqID6+CRM+Alum, applied s.c.). Samples were taken 2 weeks after 3rd application and antibody equilibrium dissociation constant (Kd) to aSyn was assessed by aSyn displacement ELISA assay.

FIG. 15 shows: the comparative analysis of in vitro functionality of immune responses elicited by CLEC based vaccines.

Female BALB/c mice, 8-12 weeks of age received a total of 3 intradermal and s.c. vaccinations applied at a 2-week interval. Samples were taken 2 weeks after 3rd application and modulation of aSyn aggregation in the presence of aSyn-specific Abs were evaluated by ThT fluorescence assays. A) aSyn was aggregated in the presence of CLEC-vaccine-induced Abs (SeqID2+SeqID7+Pus; applied i.d.), conventional peptide-component-induced Abs (SeqID3+KLH+Alum, applied s.c.) or murine plasma for 0-72 hours. B) aSyn or aSyn with pre-formed fibrils was aggregated in the presence of CLEC-vaccine-induced Abs (SeqID5+SeqID7+Pus and SeqID6+CRM+Pus, both applied i.d.), conventional peptide-component-induced Abs (SeqID6+CRM+Alum, applied s.c.) or murine plasma for 0-92 hours. Kinetic curves were calculated by normalization of ThT fluorescence at t0 and slope values extracted from linear regression analysis in the exponential growth phase of the ThT kinetic were used to calculate % inhibition of aSyn aggregation.

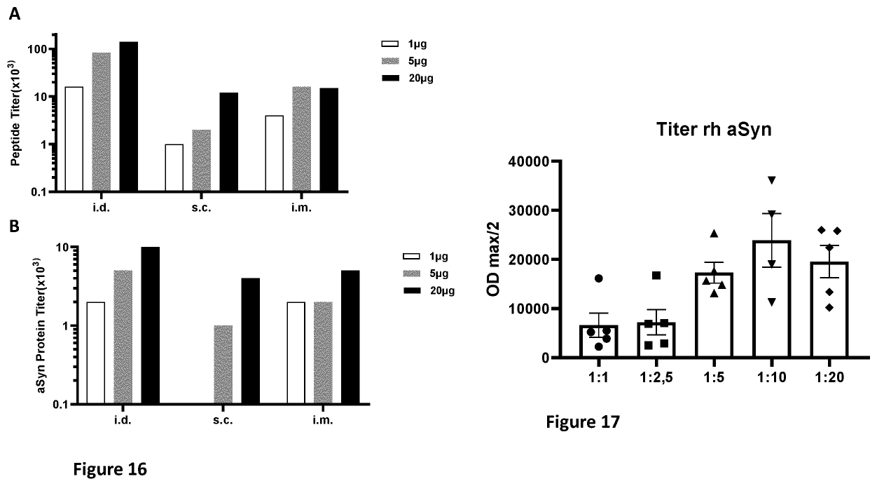

FIG. 16 shows: a comparative analysis of the effects of the route of immunization on immune responses elicited by CLEC based vaccines.

Female BALB/c mice, 8-12 weeks of age received a total of 3 vaccinations applied at a 2-week interval. Blood samples have been collected at baseline and after each vaccination to inform on the kinetics of the ensuing immune response. Two alternative routes including sub cutaneous (s.c.) and intra-muscular (i.m.) were compared to intra dermal (i.d.) application for CLEC-based vaccines. Three doses of CLEC based vaccine (SeqID2+SeqID7+Pus) were applied per route. Samples were taken 2 weeks after 3rd application and analysed for A) anti-peptide and B) anti-aSyn protein responses.

FIG. 17 shows: the Comparative analysis of the target-protein specific immunogenicity induced by CLEC-based-peptide-CRM197 conjugate vaccines using different peptide-CRM197/CLEC ratios

Female BALB/c mice, 8-12 weeks of age received a total of 3 intradermal vaccinations applied at a 2-week interval. Blood samples have been collected at baseline and after each vaccination to inform on the kinetics of the ensuing immune response. 5 different peptide-CRM-based vaccines have been used in this study applying different peptide-CRM/pustulan ratios (w/w). All 5 groups have been immunised using SeqID6+CRM+Pus conjugates. 1:1, 1:2.5, 1:5, 1:10 and 1:20 represent conjugates with a w/w peptide-CRM conjugate/CLEC ratio of 1/1, 1/2.5, 1/5, 1/10 and 1/20. Immune reactions induced have been evaluated using samples taken 2 weeks after 3rd application and analysed for anti-aSyn protein responses by ELISA. Titer determination was based on calculation of ODmax/2.

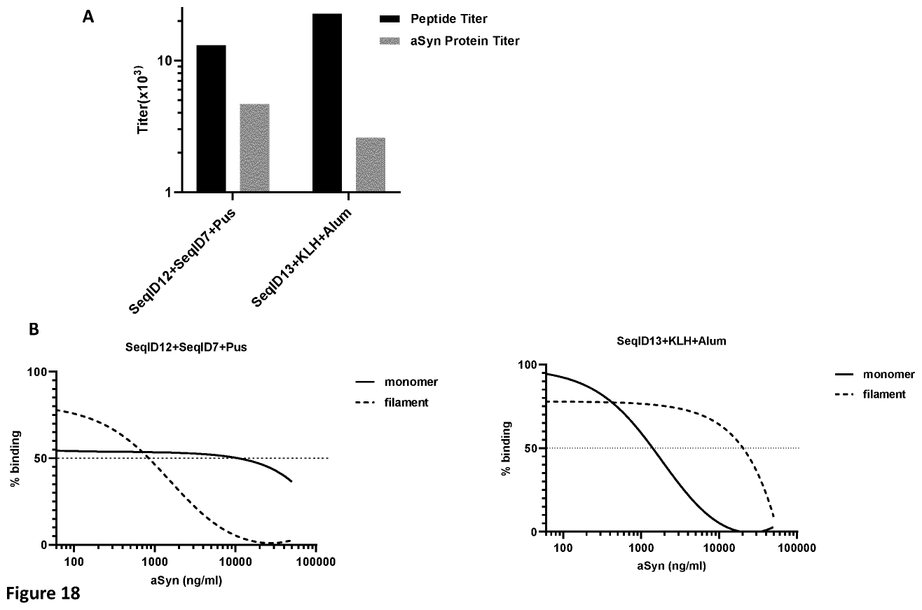

FIG. 18 shows: the comparative analysis of immune responses elicited by CLEC vaccines containing B-cell epitopes from aSyn (aa1-8).

Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID12+SeqID7+pustulan, i.d.) were evaluated against conventional peptide-component conjugate-based vaccines (SeqID13 conjugated with KLH and Alhydrogel (Alum), s.c.). Samples were taken 2 weeks after 3rd application and analysed for A) anti-peptide and anti-aSyn protein responses and B) aSyn selectivity (inhibition ELISA). Black line: monomeric aSyn used for inhibition; dashed line: filamentous aSyn used for inhibition.

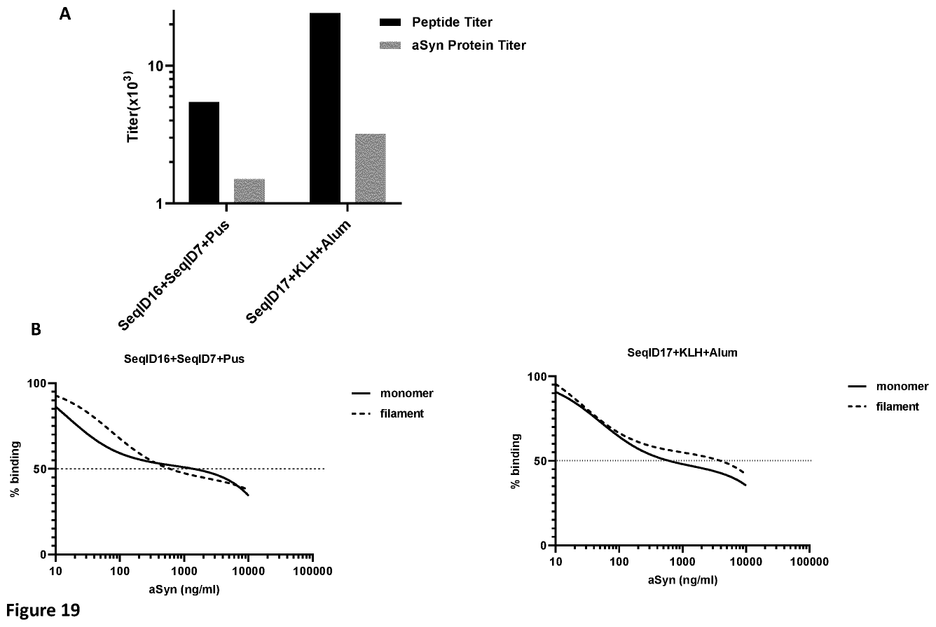

FIG. 19 shows the comparative analysis of immune responses elicited by CLEC vaccines containing B-cell epitopes from aSyn (aa100-108).

Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID16+SeqID7 and pustulan, i.d.) were evaluated against conventional peptide-component conjugate-based vaccines (SeqID17 conjugated with KLH and Alhydrogel (Alum), s.c.). Samples were taken 2 weeks after 3rd application and analysed for A) anti-peptide and anti-aSyn protein responses and B) aSyn selectivity (inhibition ELISA). Black line: monomeric aSyn used for inhibition; dashed line: filamentous aSyn used for inhibition.

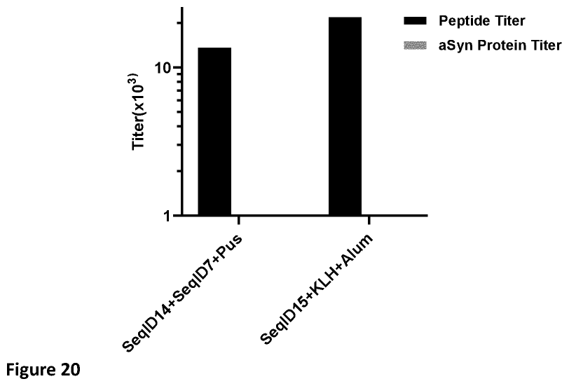

FIG. 20 shows the comparative analysis of immune responses elicited by CLEC vaccines containing B-cell epitopes from aSyn (aa91-97).

Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID14+SeqID7 and pustulan, i.d.) were evaluated against conventional peptide-component conjugate-based vaccines (SeqID15 conjugated with KLH and Alhydrogel (Alum), s.c.). Samples were taken 2 weeks after 3rd application and analysed for anti-peptide and anti-aSyn protein responses.

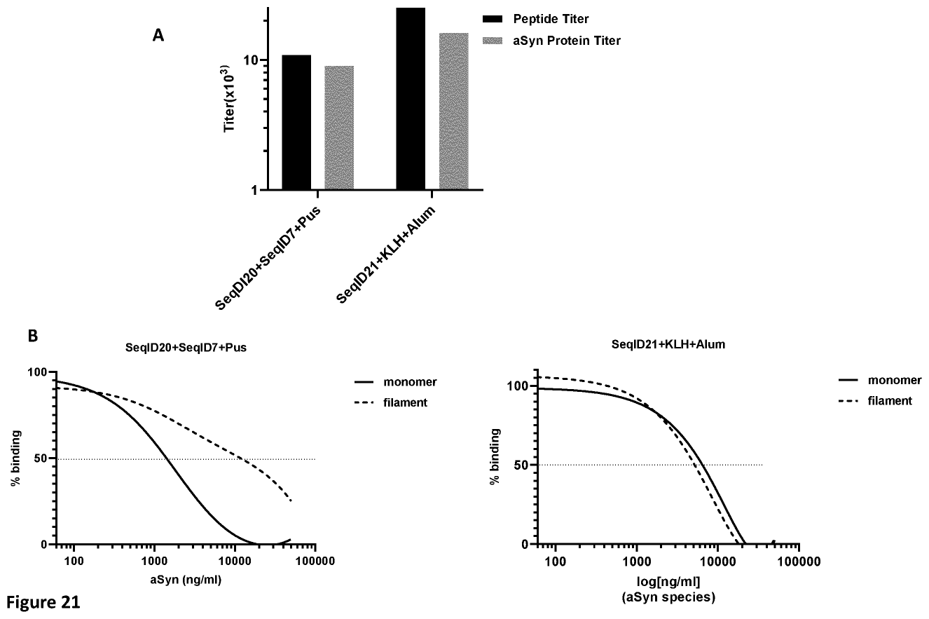

FIG. 21 shows the comparative analysis of immune responses elicited by CLEC vaccines containing B-cell epitopes from aSyn (aa130-140). Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID20+SeqID7 and pustulan, i.d.) were evaluated against conventional peptide-component conjugate-based vaccines (SeqID21 conjugated with KLH and Alhydrogel (Alum), s.c.). Samples were taken 2 weeks after 3rd application and analysed for A) anti-peptide and anti-aSyn protein responses and B) aSyn selectivity (inhibition ELISA). Black line: monomeric aSyn used for inhibition; dashed line: filamentous aSyn used for inhibition.

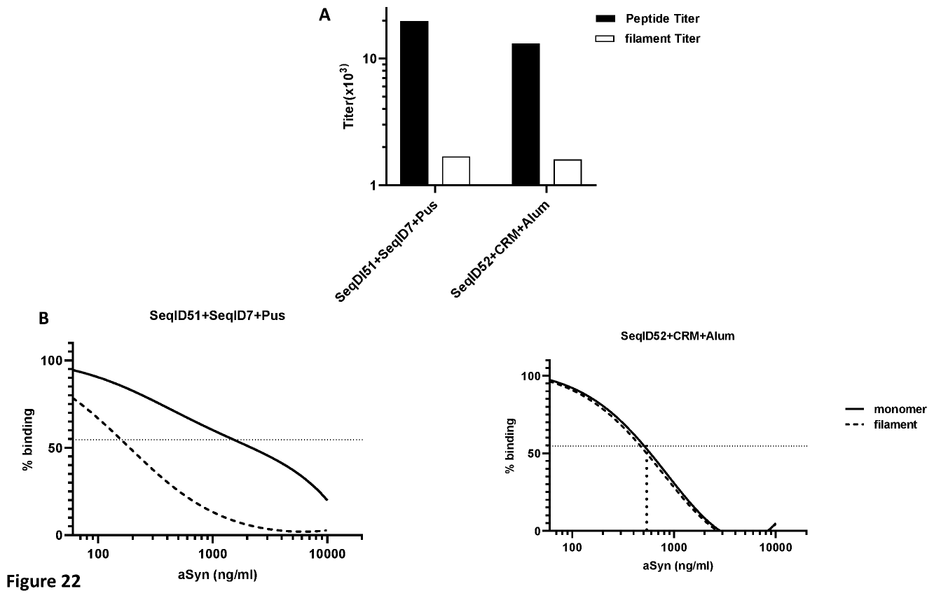

FIG. 22 shows the comparative analysis of immune responses elicited by CLEC vaccines containing B-cell epitopes from aSyn (aa115-122).

Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID51+SeqID7 and pustulan, i.d.) were evaluated against conventional peptide-component conjugate-based vaccines (SeqID52 conjugated with CRM and Alhydrogel (Alum), s.c.). Samples were taken 2 weeks after 3rd application and analysed for A) anti-peptide and anti-aSyn filament responses and B) aSyn selectivity (inhibition ELISA). Black line: monomeric aSyn used for inhibition; dashed line: filamentous aSyn used for inhibition.

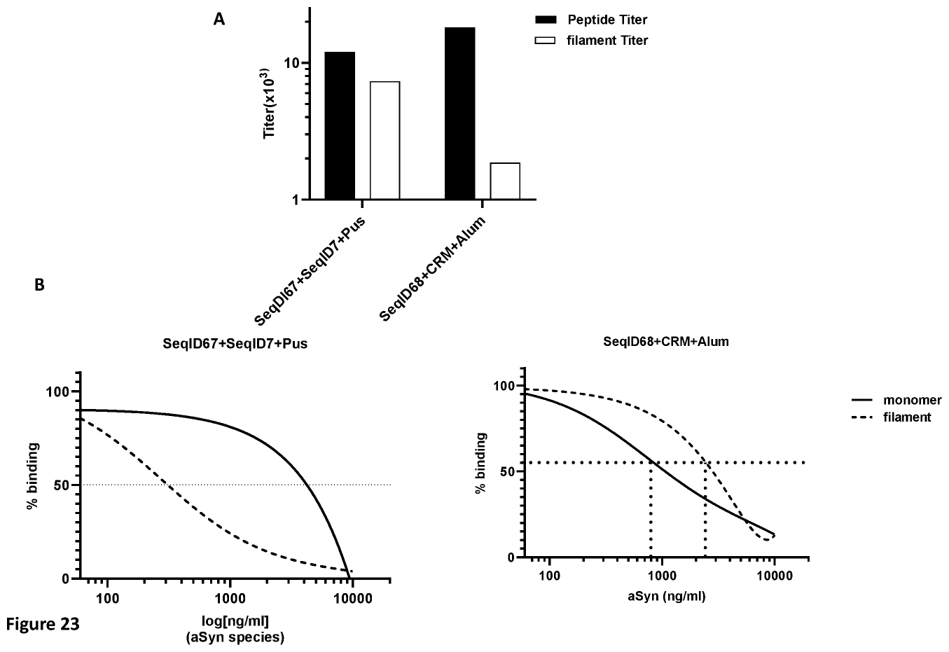

FIG. 23 shows the comparative analysis of immune responses elicited by CLEC vaccines containing B-cell epitopes from aSyn (aa115-124).

Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID67+SeqID7 and pustulan, i.d.) were evaluated against conventional peptide-component conjugate-based vaccines (SeqID68 conjugated with CRM and Alhydrogel (Alum), s.c.). Samples were taken 2 weeks after 3rd application and analysed for A) anti-peptide and anti-aSyn filament responses and B) aSyn selectivity (inhibition ELISA). Black line: monomeric aSyn used for inhibition; dashed line: filamentous aSyn used for inhibition.

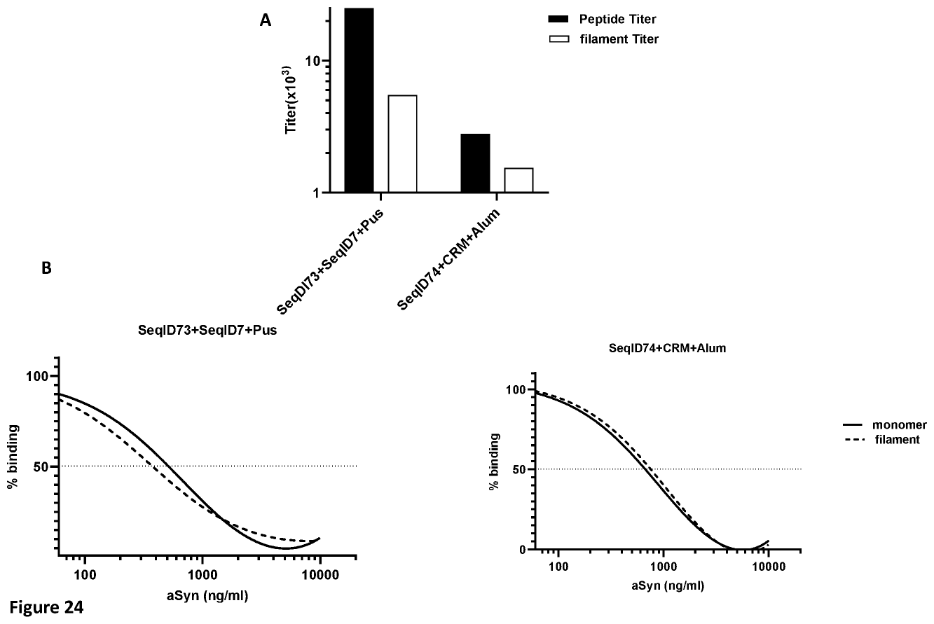

FIG. 24 shows the comparative analysis of immune responses elicited by CLEC vaccines containing B-cell epitopes from aSyn (aa107-113).

Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID73+SeqID7 and pustulan, i.d.) were evaluated against conventional peptide-component conjugate-based vaccines (SeqID74 conjugated with CRM and Alhydrogel (Alum), s.c.). Samples were taken 2 weeks after 3rd application and analysed for A) anti-peptide and anti-aSyn filament responses and B) aSyn selectivity (inhibition ELISA). Black line: monomeric aSyn used for inhibition; dashed line: filamentous aSyn used for inhibition.

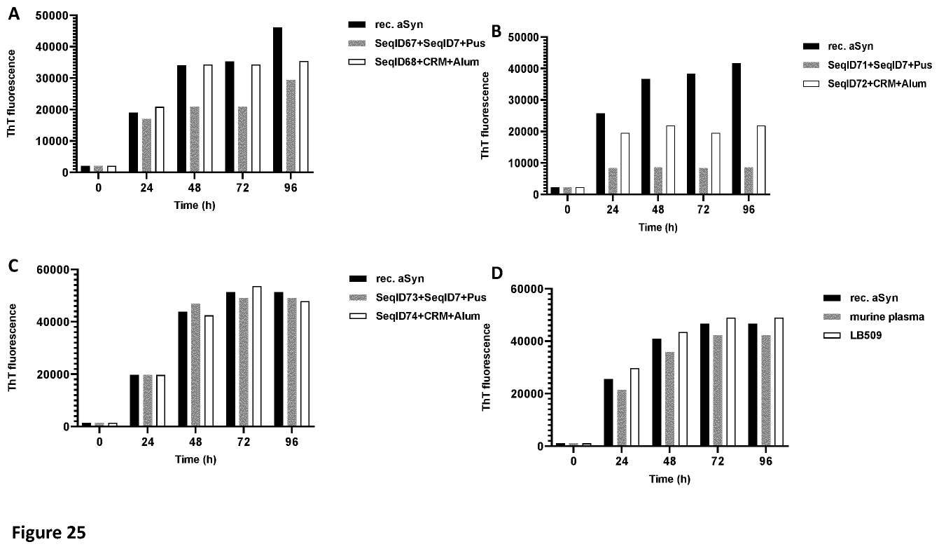

FIG. 25 shows the comparative analysis of in vitro functionality of immune responses elicited by CLEC based vaccines.

Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. Sample were taken 2 weeks after 3rd application and ThT kinetic measurements (i.e. fibrillar fraction of aSyn) were evaluated in the presence of A-C) CLEC-vaccine induced Abs (SeqID67/71/73+SeqID7 and pustulan, i.d.) or conventional peptide-component-induced Abs (SeqID68/72/74 conjugated with CRM and Alhydrogel (Alum), s.c.), or D) the aSyn specific, monoclonal Ab LB09 or untreated murine plasma.

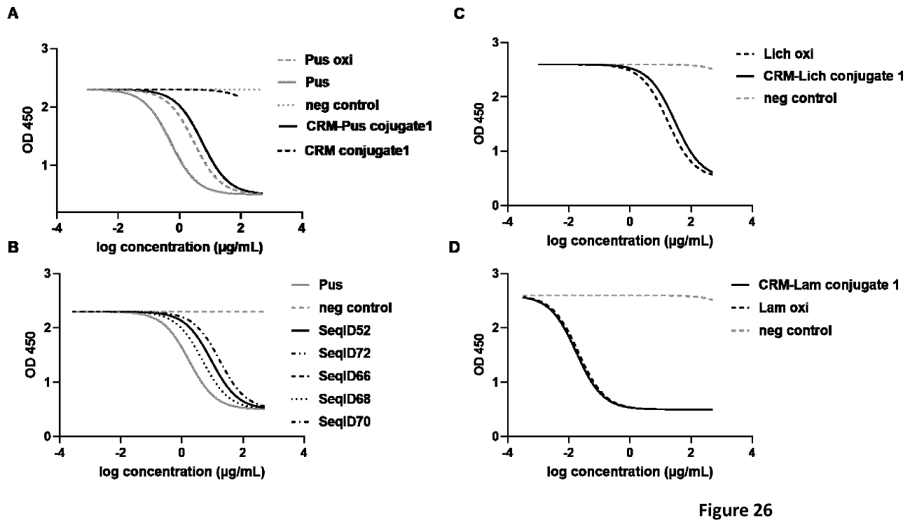

FIG. 26 shows the murine DC receptor (i.e. dectin-1) binding activity by CRM197-CLEC-conjugates in vitro.

Comparative analysis of the dectin-1 binding ability determined by ELISA is shown. A) Pus refers to non-modified pustulan and pus oxi refers to activated pustulan. CRM-pus conjugate 1 refers to the SeqID6+CRM197+pustulan conjugate and CRM conjugate 1 refers to a CRM197+SeqID6 conjugate without β-Glucan modification. Neg control refers to sample without inhibitor B) SeqID52/66/68/70/72 refer to CRM197-pustulan conjugates with indicated B-cell epitopes. C) Lich oxi refers to activated lichenan and CRM-Lich conjugate 1 refers to the SeqID6+CRM197+lichenan conjugate. D) Lam oxi refers to activated laminarin and CRM-Lam conjugate 1 refers to the SeqID6+CRM197+laminarin conjugate.

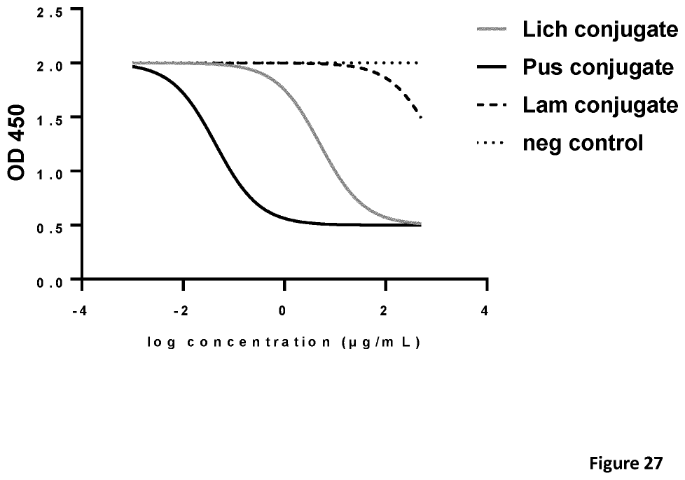

FIG. 27 shows the human DC receptor (i.e. dectin-1) binding activity by CRM197-CLEC-conjugates in vitro.

Comparative analysis of the dectin-1 binding ability determined by ELISA is shown. Lich conjugate refers to the Se-qID6+CRM197+lichenan conjugate, Pus conjugate refers to the SeqID6+CRM197+pustulan conjugate and Lam conjugate refers to the SeqID6+CRM197+laminarin conjugate. Neg control refers to sample without inhibitor.

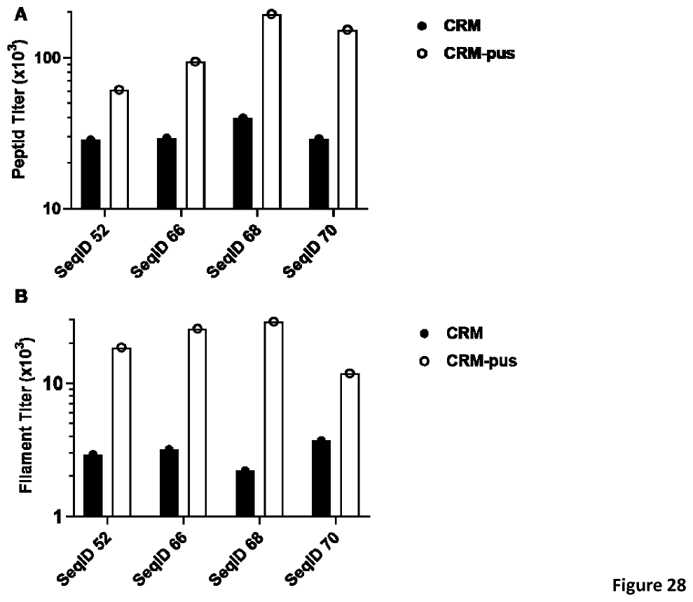

FIG. 28 shows the comparison of immunogenicity of different CRM-pustulan based vaccines.

Female BALB/c mice, 8-12 weeks of age, received a total of 3 intradermal vaccinations applied at a 2-week interval. Blood samples have been collected at baseline and after each vaccination to inform on the kinetics of the ensuing immune response. Samples were taken 2 weeks after 3rd application and analysed for A) anti-peptide response B) anti-aggregated aSyn filament responses.

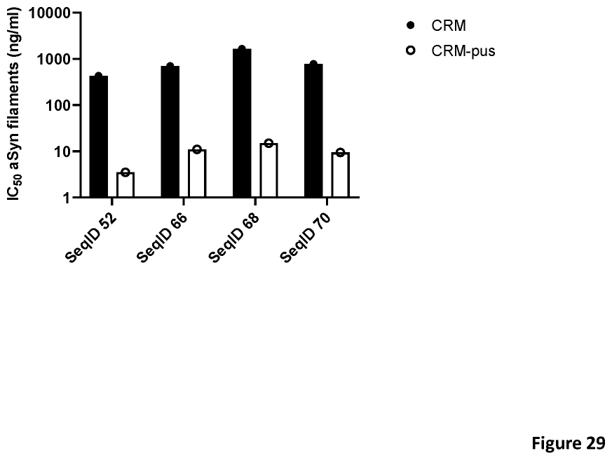

FIG. 29 shows the comparative analysis of the selectivity of the immune responses elicited by peptide+CRM+pustulan based vaccines in vivo against aSyn filaments.

Female BALB/c mice, 8-12 weeks of age received a total of 3 intradermal or s.c. vaccinations applied at a 2-week interval. CRM-pustulan based vaccine were evaluated against conventional CRM vaccine. Sample were taken 2 weeks after 3rd application and subjected to aSyn selectivity assay (inhibition ELISA). IC50 values of antibodies inhibited with increasing doses of aSyn filaments are shown.

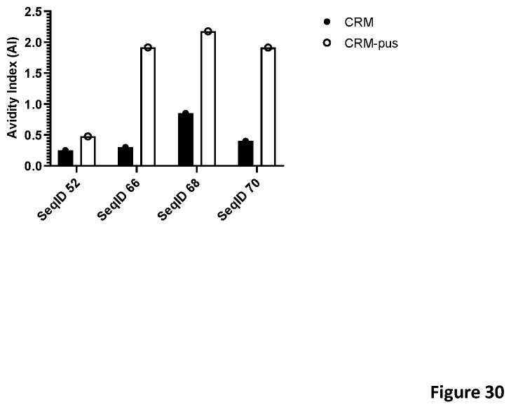

FIG. 30 shows the avidity of antibodies induced by peptide+CRM197+pustulan vaccines.

Stability of aSyn-antibody complexes induced by peptide+CRM197+pustulan- or peptide+CRM197 vaccines after challenging with different concentrations of the chaotropic agent sodium thiocyanate (NaSCN) and the determined avidity indexes are shown.

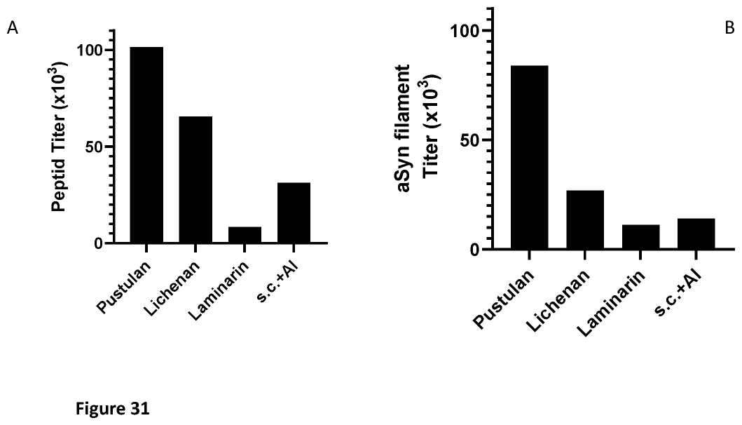

FIG. 31 shows the comparison of immunogenicity of different CLEC based vaccines.

Female BALB/c mice, 8-12 weeks of age, received a total of 3 intradermal vaccinations applied at a 2-week interval. Blood samples have been collected at baseline and after each vaccination to inform on the kinetics of the ensuing immune response. Samples were taken 2 weeks after 3rd application and analysed for anti-SeqID6 peptide response (A) and anti aSyn Filament response (B) induced by the peptide+carrier+glucan-based vaccines or the non-CLEC modified, vaccine adjuvanted with Alum; dose: 20 g peptide equivalent/injection; pustulan indicates SeqID6+CRM+pustulan, lichenan indicates SeqID6+CRM+lichenan, laminarin indicates SeqID6+CRM+laminarin, and s.c.+Alum indicates non-CLEC modified, vaccine SeqID6+CRM adjuvanted with Alum.

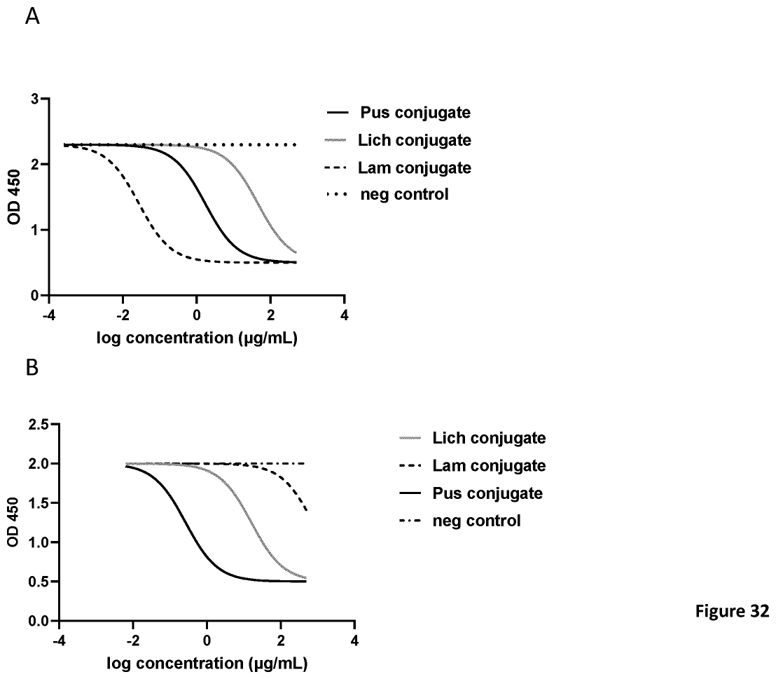

FIG. 32 shows the murine (A) and human (B) DC receptor (i.e. dectin-1) binding activity by peptide-CLEC-conjugates in vitro.

Comparative analysis of the dectin-1 binding ability determined by ELISA is shown. Lich conjugate refers to the SeqID5+SeqID7+lichenan conjugate, Pus conjugate refers to the SeqID5+SeqID7+pustulan conjugate and Lam conjugate refers to the SeqID5+SeqID7+laminarin conjugate. Neg control refers to sample without inhibitor.

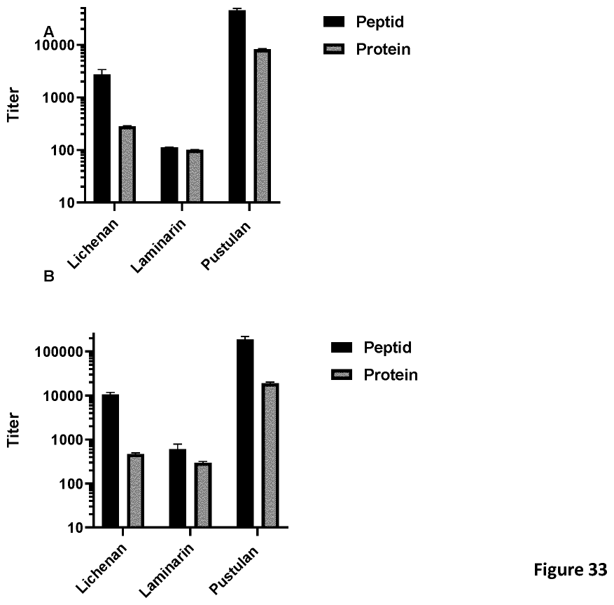

FIG. 33 shows the comparison of immunogenicity of different CLEC based vaccines.

Female BALB/c mice, 8-12 weeks of age, received a total of 3 intradermal vaccinations applied at a 2-week interval. Blood samples have been collected at baseline and after each vaccination to inform on the kinetics of the ensuing immune response. Samples were taken 2 weeks after 3rd application and analysed for anti-peptide response (SeqID6, indicated as peptide) and anti aSyn response (indicated as protein) induced by the peptide-glucan-based vaccines (i.e.: SeqID5+SeqID7+CLEC, dose: 5 μg (A) and 20 μg/injection (B); lichenan indicates SeqID5+SeqID7+lichenan; laminarin indicates SeqID5+SeqID7+laminarin and pustulan indicates SeqID5+SeqID7+pustulan)

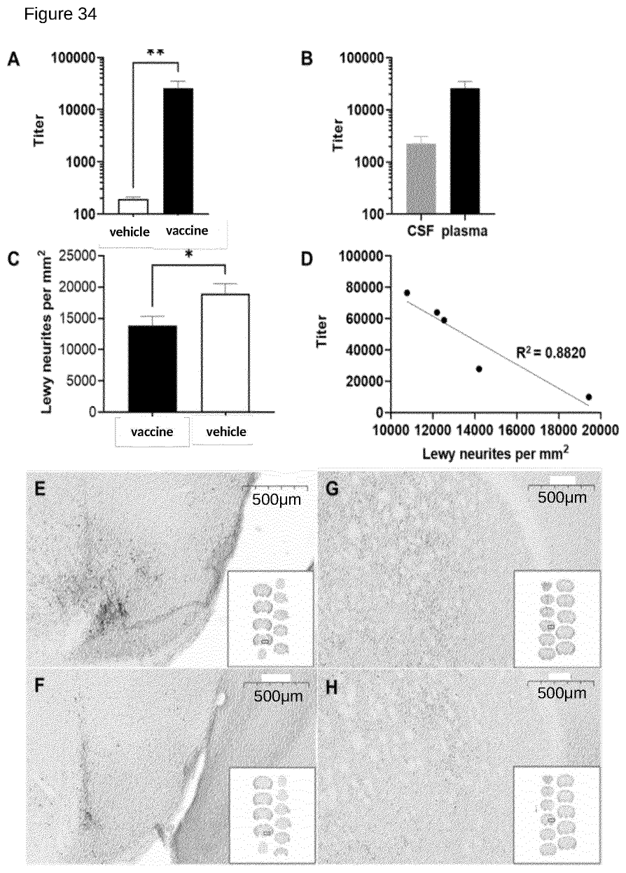

FIG. 34 shows SeqID5+SeqID7+pustulan vaccine induced antibodies inhibit aSyn aggregation in a PFF model in vivo.

C57BL/6 mice stereotactically injected in the right substantia nigra with recombinant aSyn PFFs were immunized four times using SeqID5+SeqID7+pustulan vaccine (vaccine) or unconjugated CLEC (vehicle) as control starting on the day of PFF inoculation. Plasma was collected after the third immunization. Brains, plasma and CSF were harvested 126 days post PFF inoculation. Plasma titer of antibodies specific for the peptide used for immunization (A) collected two weeks after the third immunization at day 126. (B) Comparison of CSF and plasma titers of antibodies specific for the B-cell peptide of the vaccine at day 126. (C) Analysis of phosphor-S129 aSyn-positive aggregates over all brain areas in SeqID5-SeqID7-pustulan vaccinated and CLEC treated mice. (D) Correlation between antibody response and the level of synucleinopathy in vaccine recipients (r=−0.9391; CI (95%) −0.9961 to −0.3318, p=0.0179, and R2=0.882). (E-H) Representative pSer129 aSyn staining in the injected brain hemisphere at the level of (E, F) the substantia nigra and (G, H) the striatum. (E, G) vehicle treated mice and (F, H) vaccine treated mice following PFF injection. Error bars indicate the mean±SEM of n=5-9 animals per group. Statistical differences were evaluated by an unpaired t-test; **p<0.01; *p<0.05.

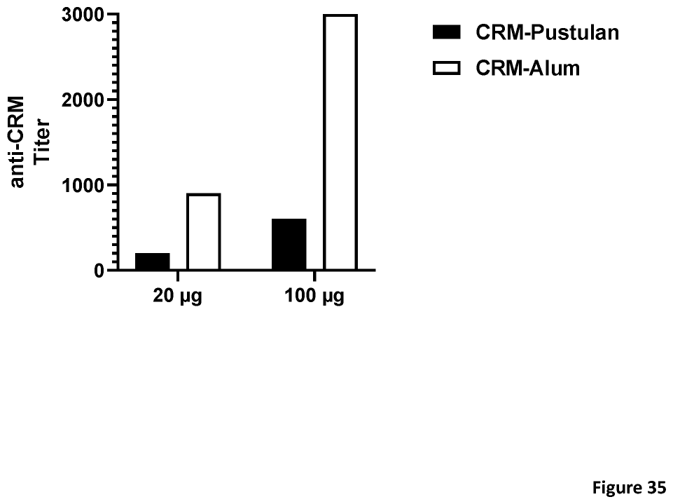

FIG. 35 shows the analysis of carrier-specific immunogenicity of Peptide+CLEC and Peptide+CRM+CLEC conjugates Female BALB/c mice, 8-12 weeks of age received a total of 3 intradermal/s.c. vaccinations applied at a 2-week interval. Blood samples have been collected at baseline and after each vaccination to inform on the kinetics of the ensuing immune response. Immune responses elicited by SeqID6+CRM197+pustulan were evaluated against the respective peptide+CRM197 conjugate adjuvanted with Alum. Samples were taken 2 weeks after 3rd application and analysed for anti-CRM responses induced in vivo.

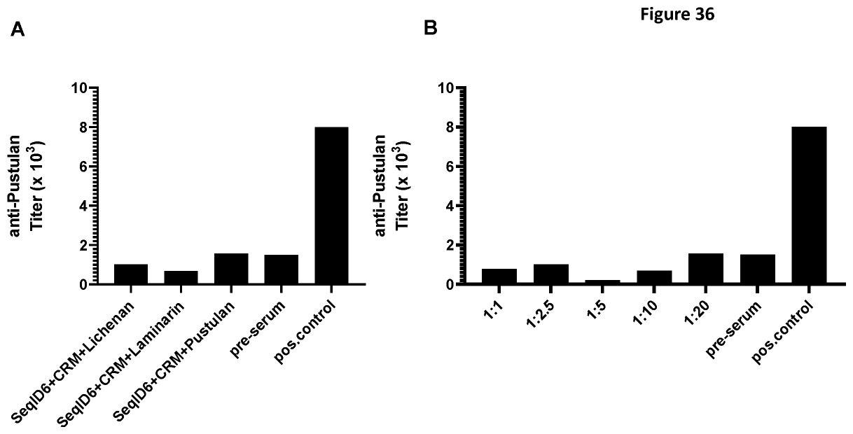

FIG. 36 shows the analysis of CLEC-specific immunogenicity of Peptide+CRM+CLEC conjugates

Female BALB/c mice, 8-12 weeks of age received a total of 3 intradermal vaccinations applied at a 2-week interval. Blood samples have been collected at baseline and after each vaccination to inform on the kinetics of the ensuing immune response. Immune responses elicited by different CLEC-based vaccines were evaluated. Samples were taken 2 weeks after 3rd application and analysed for anti-pustulan responses in vivo; A) samples: SeqID6+CRM197+pustulan, SeqID6+CRM197+lichenan; SeqID6+CRM197+laminarin B) samples SeqID6+CRM197+pustulan; pustulan coupled at indicated conjugate/pustulan ratios (w/w); pre-serum: samples obtained prior to immunisation; pos. control: samples from animals immunized with non-oxidized pustulan only.

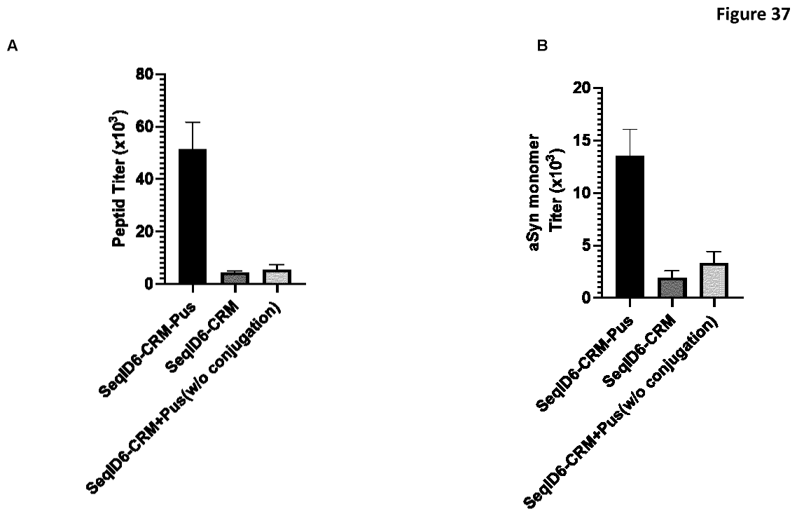

FIG. 37 shows: Analysis of the immunogenicity of peptide+carrier+glucan conjugates and vaccines consisting of peptide+carrier conjugates and unconjugated glucan.

Female BALB/c mice, 8-12 weeks of age received a total of 3 intradermal vaccinations applied at a 2-week interval. Blood samples have been collected at baseline and after each vaccination to inform on the kinetics of the ensuing immune response. Samples were taken 2 weeks after 3rd application and analysed for anti-SeqID6 peptide (A) and anti aSyn monomer (B) responses. Vaccines used: SeqID6-CRM197-pustulan: SeqID6+CRM197+pustulan, SeqID6-CRM197+Pus (w/o conjugation): non-adjuvanted SeqID6+CRM197 mixed with non-oxidized pustulan and SeqID6-CRM197: non-CLEC modified, non-adjuvanted SeqID6+CRM197.

### EXAMPLES

### Material and Methods

### 1) Oxidation of CLEC/Glucan-Backbone

For formation of vaccine conjugates, polysaccharides, especially also CLEC/β-glucans need to be chemically modified to generate reactive groups that can be used to link proteins/peptides. Two commonly used methods for polysaccharide activation are periodate oxidation at vicinal hydroxyls as well as cyanylation of hydroxyls. Further methods of activation of polysaccharides are possible and well known in the art. Examples shown in the present example section rely on mild periodate oxidation.

Depending on their solubility, CLECs and β-glucans (e.g. mannan, lichenan, pustulan or β-glucan from barley) are oxidized using periodate oxidation in aqueous solution or DMSO.

The degree of oxidation is predetermined based on adding the periodate solution at a molar ratio (periodate:sugar subunit; 100%=1 Mol periodate per Mol sugar monomers) of 1:5 (i.e., 20% oxidation) to 2.6:1 (260% oxidation degree).

Briefly, sodium periodate is added to a molar ratio of 1:5 to 2.6:1 (periodate:sugar subunit, corresponding to 20% and 260% oxidation degree) to open furanose rings of the β-glucans between vicinal diols leaving two aldehyde groups as substrate for the subsequent coupling reactions. 10% (v/v) 2-propanol is added as radical scavenger. The reaction is incubated for 4 h at room temperature on an orbital shaker (1000 rpm) in the dark. Subsequently, oxidized glucans are dialysed 3 times against water using Slide-A-Lyzer™ (Thermo Scientific) or Pur-A-Lyzer™ (Sigma Aldrich) cassettes with a 20 kDa cutoff to remove sodium (per)iodate and low molecular weight glucan impurities. Dialysed glucans can be directly subjected to the peptide conjugation reaction or stored at −20° C. or lyophilized and stored at 4° C. for further use.

### 2) Conjugation of WISIT Vaccines

2a. Via Hydrazone Formation

Polypeptides contain a hydrazide group at the N- or C-terminus for aldehyde coupling. In the case that coupling orientation is intended via the N-terminus of the selected peptide to the aldehyde groups of the glucan moieties the peptide is designed to contain a suitable linker/spacer, e.g. succinic acid. Alternatively, also intact proteins (e.g.: CRM197) have been used for glucan coupling.

Typical examples for such peptides: N-terminal coupling of peptides: H₂N—NH—CO—CH₂—CH₂—CO-Polypeptide-COOH; C-terminal coupling: NH₂—Polypeptide-NH—NH₂.

For coupling, activated glucan solution (i.e., oxidized pustulan) is stirred with the dissolved hydrazide modified peptides or intact proteins (e.g.: CRM197) in coupling buffer (depending on the isoelectric point of the peptide either sodium acetate buffer at pH 5.4, or DMEDA at neutral pH (6.8) are used). The free hydrazide group within the peptides reacts with the aldehyde group to a hydrazone bond forming the final conjugate. For proteins, coupling to activated glucan is based on reaction of the amino group of the Lysine residues present to reactive aldehydes on the glucan moieties in the presence of sodium cyanoborohydride.

Subsequently, the conjugate is reduced by addition of sodium borohydride in borate buffer (pH 8.5). This step reduces the hydrazine bond to a stable secondary amine and converts unreacted aldehyde groups in the sugar backbone into primary alcohols. Carbohydrate concentration in conjugates was estimated using anthrone method and peptide concentration was estimated by UV spectroscopy or determined by amino acid analysis.

2b. Coupling Using Heterobifunctional Linkers

The second conjugation technique applied relies on heterobifunctional linkers (e.g.: BMPH (N-β-maleimidopropionic acid hydrazide, MPBH (4-[4-N-maleimidophenyl]butyric acid hydrazide), EMCH (N-[ε-Maleimidocaproic acid) hydrazide) or KMUH (N-[κ-maleimidoundecanoic acid]hydrazide) short, maleimide-and-hydrazide crosslinkers for conjugating sulfhydryls (cysteines) to carbonyls (aldehyde)).

Polypeptides contain a cysteine (Cys) at the N- or C-terminus for maleimide coupling. Typical examples for such peptides: N-terminal coupling of peptides: Cys-Peptide-COOH; C-terminal coupling: NH₂-Pept-Cys-COOH.

For coupling, activated glucan solution (i.e., oxidized pustulan) is reacted overnight with BMPH (ratios used 1:1 ratio (w/w) to 2:1 ratio BMPH:pustulan) and subsequently dialysed 3× with PBS. BMPH-activated glucan is then mixed with the dissolved Cys-modified polypeptides in coupling buffer (e.g. phosphate-buffered saline, PBS). The maleimide group reacts with sulfhydryl groups from the peptides to form stable thioether linkages and together with the hydrazone formed between linker and reactive aldehydes results in stable conjugates. Carbohydrate concentration in conjugates was estimated using anthrone method and polypeptide concentration was determined by amino acid analysis or Ellmann's assay using Ellman's reagent (5,5′-dithio-bis-(2-nitrobenzoic acid), DTNB). DTNB reacts with sulfhydryl groups to yield a colored product, providing a reliable method to measure reduced cysteines and other free sulfhydryls in solution by spectrophotometric measurement (Δₘₐₓ=412 nm; ε=14,150/M·cm).

### 2c) Polypeptide KLH/CRM Conjugation

Polypeptides (containing N- or C-terminal Cys residues, see above) were coupled to the carrier CRM-197 (e.g.: EcoCRM, Fina Biosolutions) or KLH (Sigma Aldrich) by using the heterobifunctional crosslinking agents GMBS or SMCC (Thermo Fisher). Briefly, CRM-197/KLH was mixed with an excess of GMBS or SMCC (acc. to manufacturer's protocol) at room temperature to allow for activation, followed by removal of excess GMBS by desalting column centrifugation. Excess peptide was then added to the activated carrier for coupling (buffer: 200 mM Na-phosphate (pH=6.8)) and subsequently dialysed 3× with PBS. Coupling efficacy/peptide content was assessed using an Ellmann assay (Ellmann reagent: 5,5′-dithiobis-(2-nitrobenzoic acid) used for quantitating free sulfhydryl groups in solution). The polypeptide CRM-197/KLH conjugate was further formulated with Alum (Alhydrogel® adjuvant 2%) and applied to animals subcutaneously. Identical amounts of conjugated polypeptides were injected per mouse when the CRM-197/KLH vaccines were compared to other vaccines according to the present invention.

2d) Gluco-Neoconjugate Formation Using Polypeptide, KLH/CRM197 and glucan

Polypeptide-KLH and polypeptide-CRM197 conjugates, produced as described in 2c), were also coupled to activated glucans at different Polypeptide-KLH and polypeptide-CRM197 to Glucan ratios (i.e. 1/1 (w/w), 1/2 (w/w), 1/5(w/w), 1/10 (w/w) and 1/20 (w/w), respectively). Following polypeptide conjugate formation, Pep-KLH or Pep-CRM conjugates are reduced using Dithiothreitol (DTT). Reduced carrier-conjugates are coupled to activated glucans in the presence of an excess of heterobifunctional linker BMPH. Coupling is achieved via the maleimide group of BMPH to sulfhydryl residues of the reduced KLH or CRM197 conjugate forming a stable thioether bond and of aldehyde groups in the glycan with the hydrazide group of BMPH. After 2 hours at room temperature, the generated hydrazones are reduced to stable secondary amines by overnight incubation with sodium cyanoborohydride. Subsequently, gluconeoconjugates are dialysed 3 times against PBS or water using Slide-ALyzer™ (Thermo Scientific) or Pur-A-Lyzer™ (Sigma Aldrich) cassettes to remove low molecular weight impurities (see also: Example 23).

### 3) Determination of Biological Activity of CLEC-Conjugates In Vitro

Biological activity of mannan and glucan conjugates in vitro was analyzed by ELISA using a soluble murine Fc-dectin-1a receptor (InvivoGen) or ConA as described in Korotchenko et al., 2020. Briefly, ELISA plates are coated with a reference glucan (CLR-agonists, CLECs), e.g.: pustulan, lichenan or mannan, and are reacted with fluorescently labeled ConA (for mannan) or soluble murine Fc-dectin-1a receptor (for pustulan and other β-D-glucans), which can be detected by a HRP-labeled secondary antibody. The oxidized carbohydrates as well as the gluconeoconjugates are tested in a competitive ELISA (increasing concentration of CLECs or conjugates are added to the soluble receptors used for the assay to reduce receptor binding to coated CLECs) to demonstrate their functionality. IC₅₀ values are used to determine biological activity (i.e.: binding efficacy to soluble receptors in comparison to non-oxidised, non-conjugated ligands).

### 4) Activation Analysis Using Bone Marrow-Derived Dendritic Cells

Bone marrow-derived dendritic cells (BMDCs) were harvested from mouse femur and tibia and incubated with 20 ng/mL murine GM-CSF (Immunotools), as described in Korotchenko et al., 2020 with minor changes. Effects of various conjugates as well as of positive controls (=LPS) on CD80 and MHCII expression were assessed by FACS analysis on CD11c⁺MHCII⁺CD11b^{int} GM-CSF-derived DCs (GM-DCs).

### 5) Determination of the Hydrodynamic Radius

The hydrodynamic radius of conjugates was analyzed by dynamic light scattering (DLS). Briefly, samples (i.e., conjugates) were centrifuged at 10,000 g for 15 minutes (Merck Millipore, Ultrafree-MC-VV Durapore PVDF). All sample wells were sealed with silica oil to prevent evaporation and data was collected sequentially for approximately 24 hours. All measurements were performed with a WYATT DynaPro PlateReader-II at 25° C. in a 1536 well plate (1536W SensoPlate, Greiner Bio-One). Samples were measured in triplicate. All measurements were filtered for a baseline value of 1.00±0.005 so only curves that returned to values between 0.995 and 1.005 were considered for further analysis (e.g., cumulants radius and regularization analysis). Analysis of samples was performed according to https://www.wyatt.com/library/application-notes/by-technique/dls.html, and by DYNAMICS User's Guide (M1406 Rev. C, version 7.6.0), Technical Notes TN2004 and TN2005 (all on: www.wyatt.com)

### 6) Animal Experiments

Female BALB/c mice, n=5 mice per group, were immunized either with different CLEC conjugates (i.d., i.m., s.c.), with peptide-CRM-197/KLH conjugates (i.d.) or peptide-CRM-197/KLH conjugates adsorbed to Alum (s.c.) as well as with respective controls (e.g. unconjugated CLEC, mixture of CLEC and peptides, etc.). Animals were vaccinated 3 times in bi-weekly intervals and blood samples were taken regularly one day before each vaccination and two weeks after the last application unless differently indicated.

### 7) Quantification of Vaccine Induced Antibodies in Murine Plasma Using ELISA

Whole blood was collected from mice using heparin as anticoagulant and plasma was obtained by centrifugation. Plasma samples were stored at −80° C. To detect anti-target specific antibodies, ELISA plates (Nunc Maxisorb) were coated with peptide-BSA conjugates or recombinant proteins/fragments (usual concentration 1 μg/ml) using 50 mM sodium carbonate buffer, overnight at 4° C. All anti-polypeptide ELISA used in the examples provided are performed using Pep-BSA conjugates (e.g., SeqID3 (Sequence: DQPVLPD) with a C-terminal C for coupling to maleimide activated BSA; nomenclature: Pep1c (DQPVLPD-C, SeqID 3) is used as bait for anti-Pep1 specific responses elicited by Pep1b (SeqID2; DQPVLPD-(NH—NH₂))- and Pep1c-containing conjugate vaccines). Plates were blocked with 1% bovine serum albumin (BSA) and plasma samples were serially diluted in the plates. Detection of target specific antibodies was performed with biotinylated anti-mouse IgG (Southern Biotech) and subsequent colour reaction using Streptavidin-POD (Roche) and TMB. EC₅₀ values were calculated using GraphPad Prism software (Graph Pad Prism www.graphpad.com/scientific-software/prism/) following non-linear regression analysis (four-parameter logistic fit function).

| Target protein | Antibody |
|---|---|
| Alpha synuclein recombinant | Anti-alpha synuclein 115-121 AB |
| (Anaspec) | (LB509) (Biolegend) |
| Alpha synucuclein monomer | Anti-alpha synuclein 115-121 AB |
| (Abcam) | (LB509) (Biolegend) |
| Alpha synuclein filament | Anti-alpha synuclein 115-121 AB |
| (Abcam) | (LB509) (Biolegend) |
| Pustulan (Biozol) | |
| KLH (Sigma) | Anti-KLH AB (Sigma) |
| CRM197 (FinaBiosolution) | Anti-Diphtheria AB (Abcam) |
| Pustulan (Biozol) | |
| KLH (Sigma) | Anti-KLH AB (Sigma) |
| CRM197 (FinaBiosolution) | Anti-Diphtheria AB (Abcam) |

8) Characterization of Binding Preference of aSyn Specific Antibodies by Inhibition ELISA

ELISA plates (Nunc Maxisorb) were coated either with aSyn monomers (Abcam) or aSyn filaments (Abcam) and blocked with 1% bovine serum albumin (BSA). The control antibodies and plasma samples were incubated with serially diluted aSyn monomers or aSyn filaments in low-binding ELISA plates. Next, the pre-incubated antibodies/plasma samples were added to the monomer/filament-coated plates and detection of binding was performed with biotinylated anti-mouse IgG (Southern Biotech) and subsequent colour reaction using Streptavidin-POD (Roche) and TMB. log IC50 values were calculated as the concentration of either monomeric or filamentous aSyn needed to quench half of the ELISA signal and were used as an estimate of the Abs selectivity for the investigated antigen. log IC₅₀ values were calculated using GraphPad Prism software (Graph Pad Prism www.graphpad.com/scientific-software/prism/) following non-linear regression analysis (four-parameter logistic fit function).

9) Quantification of aSyn Aggregation

The protein aggregation assay in the automated format was carried out in a reaction volume of 0.1 ml in black, flat-bottomed 96-well plates at continuous orbital shaking in an GENIOS Microplate Reader (Tecan, Austria). The kinetics was monitored by top reading of fluorescence intensity every 20 minutes using 450-nm excitation and 505-nm emission filters. Fibril formation in the absence and presence of antibodies (antibody/protein molar ratio varied from 6×10⁻⁵ to 3×10⁻³) was initiated by shaking the aSyn solution, at a concentration of 0.3 mg/ml (20.8 μM), in 10 mM HEPES buffer (pH 7.5), 100 mM NaCl, 5 μM ThT, and 25 μg/ml heparin sulfate at 37° C. in the plate reader (Tecan, Austria).

In addition, fibril formation in the absence and presence of antibodies was also initiated by the presence of pre-formed fibrils. In brief, aSyn preformed fibrils (1 μM) were aggregated in the presence of activated aSyn monomers (10 μM) and 10 μM ThT in 100 μl PBS for 0-24 hours.

For data analysis, the mean of the negative control samples, i.e., the background fluorescence of ThT was calculated and divided from each sample at the given time point, e.g., in Microsoft Excel. To compare different conditions/inhibitors in the aggregation assay, each sample was normalized to the fluorescence reading determined at the beginning of the assay and set to 1. (t0=1).

To evaluate kinetic curves, a Michaelis Menten kinetic model was applied: Km (substrate concentration that yield a half-maximal velocity) and Vmax (maximum velocity) values of each condition were calculated using GraphPad Prism software, following enzyme kinetics analysis (Michaelis-Menten).

To compare different conditions/inhibitors in the aggregation assay, the slope value in the exponential growth phase of the ThT kinetic was calculated using GraphPad Prism software following linear regression.

### 10) Determination of Affinity and Avidity

For determination of Ab avidity, a variation of the standard ELISA assay was used where replicate wells containing antibody bound to antigens were exposed to increasing concentrations of chaotropic thiocyanate ions. Resistance to thiocyanate elution was used as the measure of avidity and an index (avidity index) representing 50% of effective antibody binding was used to compare different sera. In brief, plasma was diluted 1/500 in PBS and dispended on coated and blocked ELISA plates (Nunc Maxisorb). After incubation for 1 h, sodium thiocyanate (NaSCN, SIGMA; in PBS) was added to the samples at concentrations of 0.25 to 3 M. Plates were incubated at room temperature for 15 min prior to washing, detection and subsequent colour reaction using Streptavidin-POD (Roche) and TMB. The absorbance readings in the absence of NaSCN were assumed to represent effective total binding of specific antibody (100% binding), and subsequent absorbance readings in the presence of increasing concentrations of NaSCN were converted to the appropriate percentage of the total bound antibody. The data were fitted to a graph of (% binding) vs. (log) concentration of NaSCN and by linear regression analysis the avidity index, representing the concentration of NaSCN required to reduce the initial optical density by 50% was estimated. Data were rejected as if the correlation coefficient for the line-fitting was below 0.88.

For determination of k_{D} values (binding affinity) towards aSyn filaments, displacement ELISAs which allow a simple determination of the k_{D} value of the complex formed by an Ab and its competitive ligand were used. In brief, equal concentration of Abs were incubated with increasing concentrations of free aSyn filaments prior to measurement of free antibody titer on plates with immobilized aSyn filaments. The relative binding of Abs is expressed as a percentage of maximum binding observed in the assay for each sample; the competition reactions with aSyn filaments (5 μg/ml) were defined as representing 0% binding (unspecific binding), and reactions without competition are taken to indicate 100% (maximum) of binding in the displacement curves.

Analysis of the competition binding curves was performed according to the one-site models using the computer-assisted curve fitting software from GraphPad.

### 11) Induction of Synucleinopathy in Mice

For induction of synucleinopathy, nine-week-old male C57BL/6 mice were stereotactically injected at the level of the right substantia nigra, with preformed polymorph fibrils (PFFs; i.e., preformed sonicated T-polymorph aSyn fibrils 1B). PFFs were pre-pared and validated as described in Sci. Adv. 2020, 6, eabc4364, doi:10.1126/sciadv.abc4364; DOI: 10.1126/sciadv.abc4364. Briefly, each animal received a unilateral injection of 2 μL PFFs 1B solution (concentration: 2.5 mg/ml) into the region immediately above the right substantia nigra (coordinates from bregma: −2.9 AP, ±1.3 L and −4.5 DV) at a flow rate of 0.4 μL/min [Sci. Adv. 2020, 6, eabc4364, doi:10.1126/sciadv.abc4364; DOI: 10.1126/sciadv.abc4364] and the needle has been left in place for 5 min before being slowly withdrawn from the brain.

Starting at the same day as the inoculation, animals received three i.d. immunizations, once every two weeks (i.e., weeks 0, 2, 4), with either CLEC-based vaccine (n=5) or non-conjugated CLEC (n=10) as control, followed by a boost immunization in week 10. Upon termination of the study (day 126) cerebrospinal fluid (CSF) was collected by cisterna magna puncture and brains were carefully removed and fixed in paraformaldehyde (PFA; 4%). Coronal serial sections of the entire brain (from rostral cerebral cortex anterior to striatum to the medulla—i.e., bregma −6.72 mm) using a cryostat at 50 μm intervals were collected and processed for immunohistochemistry.

### 12) Immunohistochemistry (IHC)

IHC staining of phosphor-S129 aSyn (pS129 aSyn) on coronal serial sections was performed as previously described [Sci. Adv. 2020, 6, eabc4364, doi:10.1126/sciadv.abc4364; DOI: 10.1126/sciadv.abc4364]. The monoclonal rabbit anti-pS129 aSyn antibody EP1536Y (ab51253, Abcam) was used, followed by an incubation with labelled poly-mer-HRP anti-rabbit (Dako EnVision+TM Kit, K4011). Visualization of pS129 aSyn staining was done with Dako DAB (K3468) and sections were counterstained with Nissl stain. The actual number of pS129 aSyn aggregates per structure (cerebral cortex, striatum, thalamus, substantia nigra and brainstem) and the total number of pS129 aSyn aggregates were assessed using whole section acquisition by Panoramic Scan II (3DHISTECH, Hungary) further processed with ad-hoc developed QuPath algorithm.

### Example 1: Determination of Biological Activity of CLEC-Conjugates In Vitro

PAMPs like CLECs are recognized by PRRs present in APCs. Binding of CLECs to their cognate PRRs (e.g.: dectin-1 for β-glucans) is required to control adaptive immunity at various levels, e.g., by inducing downstream carbohydrate-specific signaling and cell activation, maturation and migration of cells to draining lymph nodes or through crosstalk with other PRRs. To provide a novel vaccine platform technology as proposed in this application, it is therefore crucial that the CLECs used are retaining their PRR binding ability, which demonstrates biological activity of the CLEC selected as well as of the CLEC based conjugate.

Along these lines and to ensure that 1) the structure of CLECs was not destroyed during mild periodate oxidation and 2) that the polysaccharide remained biologically active after coupling, binding to dectin-1 was assessed by ELISA. First, several different CLECs have been oxidized by mild periodate oxidation to produce the reactive sugar backbone of the proposed vaccines. These CLECs include: mannan, pustulan (20 kDa), lichenan (245 kDa), barley β-glucan (229 kDa), Oat β-glucan (295 kDa) and Oat β-glucan (391 kDa). Subsequently, vaccine conjugates have been produced by hydrazone coupling using different B-cell epitope peptides (SeqID2, SeqID10, SeqID16) and SeqID7 as T-helper epitope peptide, all containing a C-terminal hydrazide linker for coupling. In addition, also a peptide-pustulan conjugate produced by coupling SeqID10 via the heterobifunctional linker BMPH has been used.

Non-oxidized and oxidized CLECs as well as CLEC-based novel conjugates have then been assessed for their biological activity using a competitive ELISA system based on competitive binding of a soluble murine Fc-dectin-1a receptor (InvivoGen) or ConA as described in Korotchenko et al. 2020.

### Results:

Different CLECs tested display differential efficacy of PRR binding. In a series of ELISA experiments the dectin-1 ligands pustulan, lichenan, barley β-glucan, oat β-glucan, have been assessed for their binding efficacy to dectin-1. Ensuing experiments revealed that the median molecular weight (20 kDa), linear β-(1,6) linked β-D-glucan pustulan was surprisingly exerting significantly higher binding efficacy (ca. 3-fold) to dectin-1 than the larger, high molecular weight, linear β-(1,3) β-(1,4)-β-D glucan lichenan (ca 245 kDa) (see FIG. 1).

This difference was even more pronounced when comparing pustulan to other linear, β-(1,3) β-(1,4)-β-D glucans from oat and barley (barley β-glucan (229 kDa), oat β-glucan: 265 and 391 kd) which displayed only limited binding efficacy in comparison to pustulan (e.g.: ca. 30-fold lower for barley β-glucan (229 kDa)).

Mild periodate oxidation of selected CLECs leads to a reduction in dectin-1 binding. Oxidation of mannan reduced its binding capacity to the lectin ConA to a similar extent as the reduction described for oxidized pustulan-dectin-1 binding following periodate oxidation. Similarly, oxidation of glucans leads to a similar proportional reduction in PRR binding (see FIG. 1A).

Importantly, conjugate formation also resulted in reduction of PRR binding capacity of the peptide-CLEC conjugates compared to unconjugated CLECs, as shown for mannan-containing conjugate as well as for different pustulan, lichenan or barley and oat-β-glucan conjugates tested (see FIG. 1B).

The experiments revealed that pustulan, despite its smaller size and the absence of β-(1,3) glycosidic bonds (please note: β-(1,3) containing glucans are described as optimal ligand for dectin-1) the linear β-(1,6) linked R-D-glucan pustulan was exerting highest binding efficacy, irrespective of oxidation or conjugation. For example, pustulan containing conjugates retain an approx. 3-fold higher binding than lichenan based constructs.

With respect to IC50 values, the binding results according to FIG. 1 show binding of the various constructs to the soluble murine Fc-dectin-1a receptor. The IC50 value obtained are (FIG. 1):
Oat β-glucan 265: 860 μg/ml
Oat β-glucan 391: 820 μg/ml
Barley β-glucan 229: 145 μg/ml
Lichenan (FIG. 1E): 13 μg/ml
Lichenan 200% conjugate (FIG. 1E): 27 μg/ml (i.e. about half of unconjugated lichenan)
Pustulan: 3.5 μg/ml (FIG. 1B) and 5 μg/ml (FIG. 1D) (at least 30-fold stronger binding than the 145 μg/ml for Barley β-glucan 229)
Pustulan conjugates (FIG. 1D): 11, 14 und 15 μg/ml (i.e. about half of unconjugated pustulan)
Pustulan BMPH-Conjugate (FIG. 1F): 80 μg/ml (peptide was coupled with a heterobifunctional linker to pustulan).

FIGS. 1A and 1B further demonstrate that conjugation of peptides via hydrazone formation or via heterobifunctional linkers is equally suitable for WISIT conjugates as both types of conjugates are retaining high dectin-1 binding efficacy.

### Example 2: Determination of DC Activation Following Pustulan Exposure In Vitro

An important function of the vaccines proposed is their capacity to activate DCs following PRR binding and uptake. To demonstrate that CLEC based conjugates are not only binding to PRRs but also exert biological function in their target cells, i.e. DCs, a DC activation experiment has been performed.

First, murine bone marrow cells were incubated with mGM-CSF to generate BMDCs according to published protocols. These GM-CSF DCs were then exposed to either the peptide-glucan conjugate P SeqID2+SeqID7+pustulan or to equivalent amounts of oxidized but unconjugated sugar. In each case, conjugates/sugars were titrated from 500 μg to 62.5 μg/mL of the respective sugar. For comparison, the strong activator LPS has been used as control starting at a concentration of 2 ng/ml. Importantly, pustulan preparations used for oxidation and conjugate formation also contain small amounts of LPS. Thus, the equivalent dose of LPS was used to normalize the effects. DCs were then assessed for expression of markers for DC activation and maturation using FACS analysis including CD80 and MHCII.

### Results:

GM-CSF DCs stimulated in vitro with SeqId2-SeqID7-pustulan conjugates revealed a significantly increased expression of CD80 and MHCII (see FIG. 2). Levels were significantly higher than the effects observed by equivalent doses of LPS contained within the conjugate preparations. In contrast, equivalent amounts of oxidized but unconjugated sugar led to a slight reduction in CD80 expression as would have been expected from the LPS level in the preparation and a significantly less pronounced induction of MHCII as compared to the pustulan conjugates.

In summary, the up-regulation of MHC-II is indicative of DC activation. In addition, CD80 is upregulated more than would be expected by the same amount of LPS which strongly indicates that pustulan conjugates contribute significantly to the maturation and activation of DCs (beyond the effect explained by LPS exposure alone). Thus, examples 1 and 2 clearly demonstrate biological activity of the pustulan vaccines.

### Example 3: Particle Size Determination by DLS

Individual experiments analysing the particle size/hydrodynamic radius of different glucan conjugates have been performed.

For DLS analysis, different peptide-glucan, and peptide-carrier-glucan conjugates have been analysed and compared to non-conjugated pustulan, respectively. All analyses were performed in triplicates with a WYATT DynaPro PlateReader-II. Results obtained indicate a particle size distribution with a maximum in the low nm spectrum for all conjugates tested.

### Conjugates Tested:

| B-cell epitope | T-cell epitope | CLEC |
|---|---|---|
| SeqID2 | SeqID7 | Pustulan (80%) |
| SeqID3 | CRM197 | Pustulan (80%) |
| na | na | Non oxidized |
| | | Pustulan |

### Results:

Current analysis indicates an average main particle hydrodynamic radius (HDR) of ca. 5 nm for the peptide-pustulan conjugate SeqID2+SeqID7+pustulan used in this assay. A minor second peak detectable at ca. 60 nm indicates a very small number of aggregates present in the formulation (see FIG. 3A). Most of the conjugate preparation however seems to be present as monomeric form. This prevalence of monomeric rather than cross-linked or aggregated conjugates is also strongly supported by the fact that monomeric pustulan (ca. 20 kDa) is detectable at approx. 5 nm (as shown in the control samples see also FIG. 3C), which also supports the prevalence of monomeric pustulan conjugates (given a HDR of ˜5 nm for monomeric pustulan). As shown by Cumulants radius analysis over 24 h, the conjugates are also stable for their HDR and do not tend to aggregate again supporting the prevalence of monomeric conjugates.

To characterize vaccines based on peptide-carrier-glucan conjugates a SeqID6+CRM197 conjugate which has been additionally conjugated to pustulan was analysed. Again, DLS analysis revealed an average HDR of 11 nm and a second minor peak of ca. 75 nm again indicating the presence of a small number of aggregates (see FIG. 3B). The slight increase to 11 nm is most likely reflecting the increase of the MW of the resulting conjugate as CRM197 is around 60 kDa in size. No significant aggregation or cross linking of CRM conjugates can be detected and cumulants radius analysis over 24h also shows that the conjugates are stable for their HDR and do not tend to aggregate. Again, DLS analysis of this alternative type of CLEC based vaccines is supporting the prevalence of monomeric conjugates.

Control samples (i.e., non-oxidised pustulan) showed a much larger HDR with an average of ca. 600 nm as well as two additional smaller peaks at 5 nm and 46 nm, respectively (see FIG. 3C). Pustulan monomers have a HDR of ca. 5 nm, which fits well with the assumed MW of 20 kD, larger aggregates can be readily detected, and the majority of the glucan is present as large, high MW particles. Importantly, cumulants radius analysis over 24h also shows that, in contrast to pustulan conjugates, non-conjugated pustulan tends to strongly aggregate over time leading to the prevalent formation of large particles, consistent with various literature reports.

Example graphs for these two conjugates and non-oxidized pustulan controls are depicted in FIG. 3.

The results obtained in this example further demonstrate the so far unique characteristics of CLEC based conjugates as compared to examples well-known in the field (e.g.: Wang et al., 2019, Jin et al., 2018) with displaying small (i.e., 5-11 nm), prevalently monomeric sugar-based nanoparticles with far less than 150 nm HDR, a size which is generally considered a preferable size for immune-therapeutically active conjugate vaccines. This is mainly due to the PRR binding and activation characteristics of larger particulates including also whole glucan particles. Larger particulates (>150 nm up to 2-4 μm) are known to interact more efficiently with their receptors and can initiate DC signalling,—activation,—maturation and—migration to draining lymph-nodes whereas small, even soluble PRR-ligands are believed to be able to bind to their receptor but to block subsequent DC activation (Goodridge et al., 2011). These data, together with data described in Examples 1, 2 and 3 as well as other examples provided below however for the first time demonstrate that small, soluble peptide-based gluco-neoconjugates building on a monomeric β-glucan, e.g.: the linear β(1,6)-β-D glucan pustulan, as backbone can effectively bind to the PRR (dectin-1), activate the respective APC (as exemplified by GM-CSF DCs) and display very high biological activity and immunogenicity in skin specific manner also surpassing the effects of classical conjugate vaccines significantly.

### Example 4: In Vivo Comparison of Different CLEC-Based Vaccines

CLEC based vaccines which were able to bind to their DC receptor (e.g.: dectin-1 or ConA) were tested for their ability to induce a robust and specific immune response following repeated application in n=5 Balb/c mice/group. Typical experiments were performed applying 5 μg net peptide content of B-cell epitope peptides per dose.

In a first set of experiments three different CLECs were compared. In this experiment the aSynuclein derived peptide SeqID2 or the amyloid β 42 (Aβ42) derived peptide SeqID10 and the promiscuous T-helper cell epitope SeqID7 were coupled via C-terminal hydrazide linkers to oxidized pustulan (20% degree of oxidation), mannan (20% degree of oxidation) or barley β-glucan (229 kDa, 20% degree of oxidation).

### Vaccine Used:

| B-cell epitope | T-cell epitope | CLEC |
|---|---|---|
| SeqID2 | SeqID7 | Pustulan (20%) |
| SeqID2 | SeqID7 | Mannan (20% |
| SeqID2 | SeqID7 | barley β-glucan |
| | | (229 kDa, 20%) |
| SeqID2 | SeqID7 | Pustulan (80%) |
| SeqID2 | SeqID7 | Lichenan |
| | | (245 kDa, 200% |
| SeqID10 | SeqID7 | Pustulan (80%) |
| SeqID10 | SeqID7 | Lichenan |
| | | (245 kDa, 200% |

Animals (female Balb/c mice) were vaccinated 3 times in bi-weekly intervals (route: i.d.) and the ensuing immune response directed against the injected peptides (i.e., SeqID2 and SeqID10, respectively) was analyzed using murine plasma taken two weeks after the third immunization.

### Results:

As shown in FIG. 4A, all three CLEC vaccines (SeqID2+Se-qID7+mannan, SeqID2+SeqID7+pustulan (linear β-(1,6) β-glucan) and barley SeqID2+SeqID7+β-glucan (229 kDa) were able to induce a detectable immune response. Interestingly, immunization using the vaccine based on barley high molecular weight β-glucan did only induce a very low anti-peptide response (OD_{max/2} titer ca 1/100). In contrast, pustulan based conjugates could induce a significantly higher response with average titers of ca 1/11000. Mannan based conjugates showed a ca. 7× lower immunogenicity as compared to pustulan-based conjugates as average titers following immunization reached around 1/1500 in this experiment.

FIG. 4B displays results from a second set of experiments comparing immunogenicity of two different variants of glucan-based conjugates using either aSynuclein derived peptide SeqID2 or the amyloid β 42 (Aβ42) derived peptide SeqID10 as B-cell epitopes and the T-cell epitope SeqID7. The first variant was again relying on pustulan as CLEC for conjugation, the second variant was produced using the linear β-(1,3) β-(1,4)-β-D glucan lichenan (ca 245 kDa). As shown in FIG. 4B, both variants could induce high titer immune responses against the injected peptides (i.e. SeqID2/3 (SeqID3=SeqID2 adapted for BSA conjugation) and SeqID10/11 (SeqID11=SeqID10 adapted for BSA conjugation)). Peptide lichenan conjugates however showed a significantly lower immunogenicity than peptide pustulan conjugates in these experiments (4-8× higher anti-peptide titers at 5 μg dose) which is also in line with lower dectin-1 binding ability as shown in example 1. This demonstrates that dectin-1 binding efficacy in vitro can be directly linked to in vivo immunogenicity and biological activity of the vaccines. This leads to the identification of pustulan or fragments thereof (i.e. linear β(1,6)-β-D glucans) as most efficacious glucan variant as proposed in this application. Vaccines are also functional with different peptides demonstrating the platform potential of this vaccine type.

### Example 5: In Vivo Comparison of Peptide Pustulan Conjugates to Unconjugated Peptide Vaccines

To assess whether conjugation of CLECs to peptide immunogens is required for the induction of superior immunogenicity of the vaccines according to the present invention a set of experiments was initiated comparing two conjugates, (SeqID2+SeqID7+pustulan or SeqID2+SeqID7+mannan) to vaccine preparations containing all components as a mix without conjugation (i.e., SeqID2 and SeqID7 plus either non-oxidised pustulan or mannan, respectively). Again, n=5 female Balb/c mice were immunized i.d. three times in biweekly intervals and the ensuing immune response directed against the injected peptides (i.e., SeqID3) was analyzed using murine plasma taken two weeks after the third immunization.

### Vaccine Used:

| B-cell epitope | T-cell epitope | CLEC | Conjugation |
|---|---|---|---|
| SeqID2 | SeqID7 | Pustulan | yes |
| | | (20%) | |
| SeqID2) | SeqID7 | Mannan | yes |
| | | (20%) | |
| SeqID2 | SeqID7 | Pustulan | Non |
| | | (non- | conjugated; |
| | | activated) | mixing |
| SeqID2 | SeqID7 | Mannan | Non |
| | | (non- | conjugated; |
| | | activated) | mixing |

### Results:

FIG. 5 shows the comparison of anti-peptide (SeqID3) specific immune responses detectable following three immunizations. SeqID2+SeqID7+pustulan conjugates (20% oxidation) were able to induce 4 times higher immune responses as reported for the mix of unconjugated peptides SeqID2, SeqID7 and non-oxidized pustulan (i.e., 1/12000 vs. 1/3000) in this experiment. Similarly, also SeqID2+SeqID7+mannan conjugates (20% oxidation) were more efficient in inducing peptide specific immune responses as application of a mix of the components ( 1/7000 vs. 1/4000; 1,75-fold increase). These data show that conjugation of peptide immunogens to activated CLECs is required to induce a strong and sustainable immune response in vivo.

### Example 6: In Vivo Comparison of SeqID5+SeqID7+Pustulan and SeqID2+ or Seq-7+Pustulan Conjugates

To assess whether CLECs based vaccines require B-cell and T-cell epitopes for the induction of sustainable anti-β-cell epitope specific immune responses in vivo a set of experiments was initiated comparing three conjugates, SeqID5+SeqID7+pustulan, SeqID5+pustulan and SeqID7+pustulan. n=5 female Balb/c mice were immunized i.d. three times in biweekly intervals and the ensuing immune response directed against the injected peptides (i.e., SeqID6) was analysed using murine plasma taken two weeks after the third immunization.

| B-cell epitope | T-cell epitope | CLEC |
|---|---|---|
| SeqID5 | SeqID7 | Pustulan (80%) |
| SeqID5 | n.a. | Pustulan (80%) |
| n.a. | SeqID7 | Pustulan (80%) |

### Results:

As shown in FIG. 6, SeqID5+SeqID7+pustulan conjugates (80% oxidation) is able to induce a high and highly specific immune response directed against the injected peptide moiety (i.e., the aSynuclein derived peptide SeqID6) reaching average titers of 1/36000 in these experiments. Peptide-pustulan conjugates containing either SeqID5 or SeqID7 alone coupled to pustulan via hydrazone coupling could induce either a 12-fold lower immune response in the case of SeqID5+pustulan ( 1/3000) or no SeqID6 specific immune response (for SeqID7+pustulan conjugates, titer < 1/100, below detection limit) following three biweekly immunizations (route: i.d.).

These data show that conjugation of peptide immunogens to activated CLECs is required to induce a strong and sustainable immune response in vivo. It however also demonstrates that pustulan conjugation to individual short B-cell epitopes in the absence of T-cell epitopes (e.g.: SeqID5 alone) allows for induction of T-cell independent B-cell responses in vivo, albeit at significantly lower efficacy as reported for T- and B-cell epitope containing CLEC conjugates.

### Example 7: In Vivo Analysis of Anti-Pustulan/Glucan Immune Responses Following Peptide-Pustulan Immunisation

Analysis of anti-CLEC antibodies is important on two levels for the novelty and efficacy of the proposed CLEC-vaccines according to the present invention:
1) β-glucans are major constituents of the cell wall of various fungi, lichens and plants conferring to the cell wall its typical strength opposing intracellular osmotic pressure. β-glucans are therefore also considered typical microbial pathogen-associated molecular patterns (PAMP)s and a major target for high titer circulating natural Abs in healthy human subjects. PAMPs are common and relatively invariant molecular structures shared by many pathogens, which are powerful activators of the immune system. (Chiani et al. Vaccine 27 (2009) 513-519, Noss et al. Int Arch Allergy Immunol 2012; 157:98-108, Dong et al. J Immunol 2014; 192:1302-1312, Ishibashi et al. FEMS Immunology and Medical Microbiology 44 (2005) 99-109, Harada et al. Biol Pharm Bull. 2003 August; 26(8):1225-8). IgG to -β-(1,3)- and-β-(1,6)-glucans can be found in normal human sera and β-(1,6)-glucans appear to be much more potent antigens than β-(1-3) variants. In addition, β-(1→6)-β-glucan moiety has been identified as one of the typical microbial PAMPs, which acts as a focal point of recognition and attack for immunological malignancy surveillance, as well as defense against microbial invasion. Pustulan, the preferred glucan-backbone for the CLEC conjugates according to the present invention, is constituted from linear β-(1-6)-β-glucan moieties and it has been reported by several research groups that anti-pustulan immune responses can be detected in plasma from naïve, non-pustulan immunized human subjects. It is thus crucial to investigate the potential of CLEC based vaccines on activating anti-pustulan immunoreactivity. Anti-β-glucan antibodies could interact with peptide-pustulan specifically in vivo and could lead to the quick elimination by forming antigen-antibody complexes and thereby precluding induction of efficient immune reactions. Alternatively, induction/boosting of anti-pustulan antibody response following immunization could also foster immunogenicity as a potential cross-presentation of CLEC conjugates by anti-pustulan specific IgG antibodies and uptake into APCs could also increase the efficacy of vaccines applied.

No formal studies have been published investigating the presence of anti-pustulan antibodies in naïve mice. However, Ishibashi et al. and Harada et al. could demonstrate the existence of β-glucan IgGs to soluble scleroglucan/β-glucan (i.e., 1,3/1,6-beta-glucans) in sera of naive DBA/2 mice.
2) as previously reported (e.g.: Torosantucci et al, Bromuro et al., Donadei et al., Liao et al.) CLEC-protein conjugates, e.g. CRM197-coupled to laminarin, Curdlan or synthetic β-(1,3) β-D glucans, were acting as strong immunogens not only inducing high anti-CRM197 titers but also high anti-glucan titers combined with protection from antifungal infection. Thus, previous attempts using such conjugates have been directed at using CLECs as bona fide disease/fungal infection specific immunogens instead of using it as a carrier and immunologically inert backbone as proposed in this application.

Along these lines, an extensive analysis of plasma samples of naïve and peptide-CLEC conjugate immunized Balb/c mice (n=5/group) for the presence of anti-pustulan antibodies prior to immunization and following repeated immunizations, respectively, was initiated.

### Vaccine Used:

| B-cell epitope | T-cell epitope | CLEC | Conjugation |
|---|---|---|---|
| SeqID2 | SeqID7 | Pustulan | yes |
| | | (20%) | |
| SeqID2 | SeqID7 | Mannan | yes |
| | | (20%) | |
| | | Pustulan | Non |
| SeqID2 | SeqID7 | (non- | conjugated; |
| | | activated) | mixing |
| | | Mannan | Non |
| SeqID2 | SeqID7 | (non- | conjugated; |
| | | activated) | mixing |

### Results:

Therefore, samples from animals undergoing peptide-pustulan (SeqID2+SeqID7+pustulan (20%), and peptide-mannan (SeqID2+SeqID7+mannan (20%) immunisations were analysed (all vaccines: 4 μg of aSyn targeting peptide/dose). For control purposes also animals undergoing application of vaccines consisting of non-conjugated peptides and non-oxidised CLECs were used (i.e., SeqID2+SeqID7+non-ox. pustulan, SeqID2+SeqID7+non-ox. mannan). As shown in FIG. 7A, the Balb/c animals analysed showed a pre-existing low level immune response directed against pustulan/β(1,6)-β-D glucan. Both CLEC vaccines tested (SeqID2+SeqID7+pustulan (20%), and SeqID2+SeqID7+mannan (20%)) failed in inducing a strong de novo immune responses directed against the glucan backbone in vivo. In contrast, repeated application of unconjugated, non-oxidised pustulan present in the control group (receiving a mix of all three components) led to the induction of a strong anti-glucan immune response by boosting antibody levels against pustulan 18.5 times (compared to pre-immune plasma). Mannan containing conjugates or mixes were unable to induce anti-pustulan titers indicating specificity of the anti-glucan response detected. Kinetic analysis of anti-pustulan antibody titers showed a steady increase over time with a strong increase after the third immunization in animals undergoing immunization using non-conjugated and non-oxidised pustulan (see FIG. 7B). A competition ELISA using increasing amounts of native pustulan also demonstrated the specificity of the antibody response detectable in the group receiving a mix of components (FIG. 7C).

In summary, these analyses could demonstrate that: despite presence of a low-level auto-reactivity against pustulan (IgG) in naïve Balb/c mice, no/very low vaccination dependent increase of anti-pustulan immunoreactivity is induced by immunization using various CLEC conjugates. Therefore, the CLECs as used as peptide-CLEC conjugates according to this invention are indeed immunologically inert using the novel vaccine design according to the present invention. This is in strong contrast to previously published results and therefore constitutes a surprising and inventive novel characteristic of the carbohydrate backbone (e.g. the β-glucans or mannans, especially the pustulan backbone) according to the present invention.

In addition, pre-existing anti-pustulan-responses do not seem to preclude immune reactions to the peptide component of WISIT vaccines as the injected peptide responses for both experiments revealed high anti-peptide titers.

### Example 8: Analysis of Immunogenicity of Glucan Conjugates with N- or C-Terminally Coupled Peptide Immunogens

To assess whether linker orientation used for coupling would interfere with immunogenicity of the vaccines, 4 different vaccine candidates were produced: In this experiment the aSynuclein derived peptides SeqID1/2 and SeqID4/5 were coupled either via N- or C-terminal hydrazide linkers to oxidized pustulan (80%). In addition, each of the 4 vaccines was carrying the promiscuous T-helper cell epitope SeqID7 coupled via C-terminal hydrazide linkers to the CLEC backbone.

### Vaccine Used:

| B-cell epitope | T-cell epitope | CLEC |
|---|---|---|
| SeqID1 | SeqID7 | Pustulan (80%) |
| SeqID2 | SeqID7 | Pustulan (80%) |
| SeqID4 | SeqID7 | Pustulan (80%) |
| SeqID5 | SeqID7 | Pustulan (80%) |

Animals (female Balb/c mice) were vaccinated 3 times in biweekly intervals (route: i.d.) and the ensuing immune response directed against the injected peptide (i.e., SeqID3 and SeqID6) as well as against the target protein, i.e. recombinant aSynuclein was analyzed using murine plasma taken two weeks after the third immunization.

### Results:

As shown in FIG. 8, all 4 CLEC vaccines using either N- or C-terminally coupled B-cell epitopes were able to induce a strong and highly specific immune response against both, the injected peptide moieties (FIG. 8A), and the target protein: aSynuclein (FIG. 8B). Interestingly, coupling orientation was differentially affecting immunogenicity. For example, the N-terminally coupled SeqID1+SeqID7+CLEC vaccine was inducing a 7-fold lower anti-injected peptide response and a 10-fold lower immune response directed against rec. aSyn as the C-terminally coupled SeqID2+SeqID7+CLEC, respectively. In contrast C-terminally coupled SeqID5+SeqID7+CLEC vaccine was inducing an approx. 4-fold lower injected peptide response but an equal anti-aSyn response as compared to N-terminally coupled SeqID4+SeqID7+CLEC vaccine, respectively. Thus, coupling orientation can be changed based on peptide specific characteristics without affecting the development of high titer immune responses.

However, as shown in FIG. 8, by variation of the coupling orientation of the immunogenic peptide the specificity of the ensuing response against the target protein can be significantly increased and can thus be used to create novel and unprecedented immune responses:
e.g.: SeqID1 vaccination leads to a 4.5-fold higher response against the peptide as compared to the target protein which is comparable to the 3,3-fold higher anti-peptide response as compared to the protein induced by the SeqID 2 vaccine.

In contrast SeqID4 vaccine induces a 1.7-fold higher response against the peptide as compared to the protein, whereas the SeqID5 vaccine could reverse this ratio leading to a 2.5-fold higher protein specific response as compared to the injected peptide response detectable.

In summary, these data clearly show that vaccines using either coupling direction are biologically active and are suitable for this application. It is also shown that coupling orientation can be used to elect specifically preferred and unprecedently active vaccines depending on the peptide and target to be addressed.

### Example 9: Analysis of Immunogenicity of CLEC Conjugates Using Different T-Helper Cell Epitopes

In this example immunogenicity of CLEC based vaccines containing the nonnatural pan DR epitope (PADRE containing an artificial Cathepsin cleavage site, SeqID7) were compared to other well-known T-helper cell epitopes. For this purpose, several promiscuous epitopes have been selected which have either been adapted using a novel, artificially included Cathepsin L cleavage site for efficient endo/lysomal release following receptor mediated uptake in APCs/DCs or left unchanged. The epitopes selected include:

| | | | Cathepsin |
|---|---|---|---|
| | | | L cleavage |
| Peptide | Sequence | Epitope | site |
| SeqID7 | AKFVAAWTLKAAANRRA-(NH-NH₂) | PADRE | + |
| SeqID22 | AKFVAAWTLKAAA-(NH-NH₂) | PADRE | − |
| SeqID23 | KAAAVKAAFWTAL-NRRA-(NH-NH₂) | artificial | + |
| SeqID24 | DSETADNLEKTVAALSILPGHGC-(NH-NH₂) | Diphtheria | − |
| SeqID25 | DSETADNLEKTVAALSILPGHGCNRRA- | Diphtheria | + |
| | (NH-NH₂) | | |
| SeqID26 | ISITEIKGVIVHRIETILF-(NH-NH₂) | Measles | |
| | | Virus | |
| | | fusion | − |
| | | protein | |
| SeqID27 | ISITEIKGVIVHRIETILENRRA-(NH-NH₂) | Measles | |
| | | Virus | |
| | | fusion | + |
| | | protein | |
| SeqID28 | ISQAVHAAHAEINEAGR-(NH-NH₂) | Chicken | − |
| | | OVA | |
| | | (323-339) | |
| SeqID29 | ISQAVHAAHAEINEAGRNRRA-(NH-NH₂) | Chicken | + |
| | | OVA | |
| | | (323-339) | |

To assess whether peptide vaccines carrying these T-helper cell epitope peptides could mount high immune reactions following repeated immunization and could induce immune reactions which were superior to conventional conjugate vaccines, 10 different vaccine candidates were tested:

In this experiment the aSyn derived peptide SeqID2 was either used as peptide-CLEC vaccine (i.e.: SeqID2, in combination with the different T-helper cell epitopes coupled via C-terminal hydrazide linkers to oxidized pustulan (80%;)) or a conventional peptide-conjugate was produced using SeqID3 containing a C-terminal cysteine for coupling to GMBS activated KLH.

### Vaccine Used:

| | | T-cell | | | |
|---|---|---|---|---|---|
| | B-cell | epitope/ | | | |
| Vaccine | epitope | carrier | CLEC | adjuvant | Route |
| 1 | SeqID2 | SeqID7 | Pustulan | n.a | i.d. |
| | | | (80%) | | |
| 2 | SeqID2 | SeqID22 | Pustulan | n.a | i.d. |
| | | | (80%) | | |
| 3 | SeqID2 | SeqID23 | Pustulan | n.a | i.d. |
| | | | (80%) | | |
| 4 | SeqID2 | SeqID24 | Pustulan | n.a | i.d. |
| | | | (80%) | | |
| 5 | SeqID2 | SeqID25 | Pustulan | n.a | i.d. |
| | | | (80%) | | |
| 6 | SeqID2 | SeqID26 | Pustulan | n.a | i.d. |
| | | | (80%) | | |
| 7 | SeqID2 | SeqID27 | Pustulan | n.a | i.d. |
| | | | (80%) | | |
| 8 | SeqID2 | SeqID28 | Pustulan | n.a | i.d. |
| | | | (80%) | | |
| 9 | SeqID2 | SeqID29 | Pustulan | n.a | i.d. |
| | | | (80%) | | |
| 10 | SeqID3 | KLH | n.a | Alhydrogel | s.c. |

Animals (female Balb/c mice) were vaccinated 3 times in biweekly intervals (all vaccines: 5 μg of aSyn targeting peptide/dose; route: i.d. for the CLEC based vaccine and s.c. for the KLH based vaccine (adjuvanted with Alhydrogel) and the ensuing immune response directed against the injected peptide (i.e., SeqID3) as well as against the target protein, i.e., recombinant human aSynuclein has been analysed using murine plasma taken two weeks after the third immunization.

### Results:

As shown in FIG. 9, all 9 CLEC vaccines using different T-helper-cell epitopes and the KLH conjugate were able to induce a strong and specific immune response against both, the injected peptide moieties (SeqID3, FIG. 9A) and the target protein: recombinant aSynuclein (FIG. 9B).

All T-helper epitopes could induce anti-peptide titers similar or superior to the conventional SeqID3+KLH conjugate. Vaccine 1 (containing SeqID2 and SeqID7 coupled to pustulan) for example could induce a 60% higher response as the KLH control, whereas Vaccine 8 (containing SeqID28, a well well-known T-helper epitope specifically suitable for application in Balb/c animals, SeqID2 and pustulan) could induce a 5.5-fold higher response than the control. Even the promiscuous T-helper epitope SeqID24 (derived from Diptheria Toxin, a weak T-helper epitope for Balb/c animals disclosed in WO 2019/21355 A1) was able to induce a sustainable immune response, albeit weaker than the KLH control.

Similarly, all T-helper epitopes could induce anti-protein titers similar or superior to the conventional SeqID3-KLH conjugate. Importantly, Vaccine 1 (containing SeqID2 and SeqID7 coupled to pustulan) for example could induce a 2.5-fold higher response as the KLH control, and Vaccine 8 (containing SeqID28, a well well-known T-helper epitope specifically suitable for application in Balb/c animals, SeqID2 and pustulan) could induce a 3-fold higher response than the control again supporting the fact that CLEC based vaccines according to this invention can induce superior anti-target responses.

The example also shows that introduction of an additional Cathepsin L cleavage site to the well described T-helper epitopes leads to more efficient induction of immune responses as compared to conventional vaccines and to CLEC based vaccines devoid of this artificial sequence.

For example, SeqID25, the modified variant of the weak T-helper epitope SeqID24 containing the cleavage site, was able to induce a 7.5-fold higher anti-peptide and a 3.6-fold higher anti-protein response as compared to the unmodified peptide (vaccine 5 vs. vaccine 4). In addition, this alteration also led to a 40% increase in anti-protein titers as compared to the KLH control. SeqID27, the Cathepsin L cleavage site modified variant of SeqID26 (an epitope derived from Measles virus fusion protein, disclosed in in WO 2019/21355 A1) could also significantly augment titers with a 1.8-fold increase in anti-peptide and a 3.2-fold increase in anti-protein titers as compared to the SeqID26-CLEC vaccine (i.e., vaccine 7 vs. vaccine 6). Vaccine 7 was also inducing a 2.2-fold higher anti-peptide response and a 1.6-fold higher anti-protein response as the KLH control. SeqID7 based CLEC vaccines are also inducing superior anti-protein titers (20% increase) as compared to non-modified variants (e.g.: SeqID22) and both peptides lead to an approximate doubling of anti-SeqID2 peptide and anti-aSyn titers as compared to the KLH control, respectively.

Addition of the Cathepsin cleavage site leads to formation of a peptide variant with an additional N (e.g.: at the C-terminus released upon cleavage. E.g.: SeqID22, PADRE, is released as AKFVAAWTLKAAA whereas SeqID7, modified PADRE is released as AKFVAAWTLKAAA-N. This N could also negatively impact further processing and MHCII presentation and could thus lower efficacy of the respective peptide. This phenomenon can be seen at the example of the very strong OVA derived epitopes SeqID28 and SeqID29. The unmodified peptide is inducing very high immune responses whereas the modified variant pep17 induces a 75% reduced anti-peptide and a 98% reduced anti-protein titer as compared to the unmodified variant.

### Example 10: Analysis of Immunogenicity of CLEC Conjugates Using Carrier Proteins as T-Helper Cell Epitopes: KLH

In this example immunogenicity of CLEC based conjugate vaccines containing the well-known carrier protein KLH was compared to conventional KLH vaccines. For this purpose, two aSyn derived epitopes (SeqID3 and SeqID6) have been selected which have been coupled to GMBS activated KLH. Subsequently, Pep-KLH conjugates have been coupled to reactive aldehydes of oxidized pustulan using the BPMH crosslinker to form CLEC based conjugate vaccines with KLH as source for T-helper cell epitopes to induce a sustainable immune response.

| | T-cell | | | |
|---|---|---|---|---|
| B-cell | epitope/ | | | |
| epitope | carrier | CLEC | adjuvant | Route |
| SeqID3 | KLH | Pustulan | n.a | i.d. |
| | | (140%) | | |
| SeqID3 | KLH | n.a | n.a | i.d |
| SeqID3 | KLH | n.a | Alhydrogel | s.c. |
| SeqID6 | KLH | Pustulan | n.a | i.d. |
| | | (80%) | | |
| SeqID6 | KLH | n.a | n.a | i.d. |
| SeqID6 | KLH | n.a | Alhydrogel | s.c. |

Animals (female Balb/c mice) were vaccinated 3 times in biweekly intervals (all vaccines: 20 μg of aSyn targeting peptide/dose; route: i.d. for the CLEC based vaccine and for non-adjuvanted KLH based vaccine and s.c. for the KLH based vaccine adjuvanted with Alhydrogel) and the ensuing immune response directed against the injected peptide (i.e. SeqID3 and SeqID6) as well as against the target protein, i.e. recombinant human aSynuclein has been analysed using murine plasma taken two weeks after the third immunization.

### Results:

As shown in FIG. 10A, all 6 vaccines using KLH as source for T-helper epitopes were able to induce a strong and specific immune response against both, the injected peptide moieties (SeqID3 and SeqID6) and the target protein: recombinant aSynuclein.

CLEC modification of the KLH conjugates lead to a highly superior immune response using both peptides, SeqID3 and SeqID6, respectively. SeqID3+KLH+pustulan was able to induce 2.3 times higher anti-peptide responses as Alhydrogel adjuvanted SeqID3+KLH and a 14 times higher response as obtained following i.d. application of non-adjuvanted SeqID3+KLH. Similarly, also anti-protein titers were 8.5-fold increased (compared to Alhydrogel adjuvanted SeqID3+KLH) and 17 times as compared to non-adjuvanted material. SeqID6+KLH+pustulan was also 2 (inj. peptide) to 4.6 times (alpha synuclein) more effective than adjuvanted SeqID6+KLH and 8.7 (inj. peptide) and 11 times (alpha synuclein) more immunogenic than the non-adjuvanted SeqID6+KLH vaccine, respectively.

Besides a general increase in immunogenicity of CLEC modified vaccines, the results also show that CLEC modification according to this invention leads to a significant increase in the relative amount of antibodies induced which are binding to the target molecule, i.e., the protein thereby increasing target specificity of the ensuing immune response significantly. Accordingly, the relative amount of antibodies detecting alpha synuclein (i.e., the ratio of total anti-injected peptide titers compared to anti-alpha synuclein specific titers) is 3.7 times higher for SeqID3+KLH+pustulan induced responses as compared to adjuvanted SeqID3+KLH and 2.2 times higher in the case of SeqID6+KLH+pustulan as compared to adjuvanted conjugates.

In a second set of experiments, the same vaccines used (all vaccines: 5 μg of aSyn targeting peptide/dose; route: i.d. for the CLEC based vaccine and s.c. for the KLH based vaccine adjuvanted with Alhydrogel) were compared for their ability to induce anti-carrier specific antibody responses. As expected, conventional SeqID3+ and SeqID6+KLH based vaccines were able to induce high anti-KLH titers (SeqID3+KLH: 1/2100 and SeqID6+KLH: 1/7700) whereas the CLEC based SeqID3+KLH+pustulan and SeqID6+KLH+pustulan vaccines were basically unable to induce sustainable anti-carrier antibodies. The titers obtained were close to the detection limit with 1/150 for SeqID3+KLH+pustulan and less than 1/100 for SeqID6+KLH+pustulan respectively thus creating a novel, yet undescribed optimization strategy for peptide-conjugate vaccines to increase target specific titers while reducing unwanted anti-carrier responses.

### Example 11: Analysis of Immunogenicity of CLEC Conjugates Using Carrier Proteins as T-Helper Cell Epitopes: CRM197

In this example immunogenicity of CLEC based conjugate vaccines containing the well-known carrier protein CRM197 was compared to conventional CRM197 vaccines. For this purpose, the alpha synuclein derived epitope SeqID6 has been coupled to maleimide activated CRM197. Subsequently, SeqID6+CRM197 conjugate has been coupled to activated pustulan using the heterobifunctional linker BPMH to form CLEC based conjugate vaccines with CRM197 as source for T-helper cell epitopes to induce a sustainable immune response. Alternatively, SeqID5-(NH—NH2; SeqID5) and CRM197 have been coupled to activated pustulan, independently. This was done by reaction of the hydrazide at the C-terminus of SeqID5 and via Lysins present in CRM197 to reactive aldehydes on activated pustulan.

### Vaccines Used:

| | T-cell | | | | |
|---|---|---|---|---|---|
| B-cell | epitope/ | | CLEC | | |
| epitope | carrier | CLEC | coupling | adjuvant | Route |
| SeqID6 | CRM197 | Pustulan | as conjugate | n.a | i.d. |
| | | (80%) | | | |
| SeqID5 | CRM197 | Pustulan | independent | n.a | i.d. |
| | | (80%) | | | |
| SeqID6 | CRM197 | n.a | n.a | Alhydrogel | s.c. |

Animals (female Balb/c mice) were vaccinated 3 times in biweekly intervals (all vaccines: 20 μg of alpha synuclein targeting peptide/dose; route: i.d. for the CLEC based vaccines and and s.c. for the CRM197 based vaccine adjuvanted with Alhydrogel) and the ensuing immune response directed against the injected peptide (i.e., SeqID6) as well as against the target protein, i.e. recombinant human alpha synuclein as well as alpha synuclein filament has been analysed using murine plasma taken two weeks after the third immunization.

### Results:

As shown in FIG. 11A, all 3 vaccines using CRM197 as source for T-helper epitopes were able to induce a strong and specific immune response against both, the injected peptide moieties (SeqID6) and the target protein: recombinant alpha synuclein.

Again, CLEC modification of the CRM197 conjugates led to a highly superior immune response. SeqID6+CRM197+pustulan was able to induce 28 times higher anti-peptide responses as Alhydrogel adjuvanted SeqID6+CRM197. Similarly, also anti-protein titers against recombinant aSyn were 15-fold increased (compared to Alhydrogel adjuvanted SeqID6+CRM197) and titers against the aggregated form of alpha synuclein, alpha synuclein filaments, was 11-fold increased.

The vaccine produced by independently coupling SeqID5 and CRM197 to pustulan was also inducing 1.7 times higher inj. peptide titers as conventional Alhydrogel adjuvanted SeqID6+CRM197. Reactivity to recombinant alpha synuclein was also increased 6.6 times and anti-filament responses were increased by a factor of 4.25, respectively.

Comparison of anti-carrier specific antibody responses revealed that conventional SeqID6+CRM197 based vaccines were able to induce high anti-CRM197 titers ( 1/6600) whereas the CLEC based SeqID6+CRM197+pustulan vaccine was basically unable to induce sustainable anti-carrier antibodies. The titers obtained were close to the detection limit with less than 1/100 for SeqID6+CRM197+pustulan respectively.

Thus, the experiments show that CLEC modification of conventional peptide-protein conjugates impairs development of an anti-carrier response significantly and leads to a strongly enhanced target specificity of the ensuing immune response providing a novel unprecedented strategy to optimize current state of the art conjugate vaccines building on carrier proteins like KLH, CRM197 or others.

Independent coupling of CRM197 and SeqID6 to pustulan leads to sustainable response against the B-cell epitopes present on CRM197, although at a lower rate as detectable for conventional, non-CLEC modified conjugates (Titer ca. 1/400). This shows that the CLEC backbone according to the current invention is also suitable to provide B-cell epitopes from CLEC coupled immunogenic proteins for use as vaccine.

### Example 12: Analysis of Selectivity of Immune Responses Elicited by CLEC Based Vaccines In Vivo

Aggregation of the presynaptic protein aSyn has been implicated as major pathologic culprit in synucleinopathies like Parkinson's disease whereas monomeric, non-aggregated aSyn has important neuronal functions. It is thus believed to be crucial for treatment of synucleinopathies, for example by active or passive immunotherapy, to reduce/remove aggregated aSyn without affecting the available pool of non-aggregated molecules present.

To further characterize the immune responses elicited by CLEC based vaccines containing the aSyn targeting peptides SeqID2 and SeqID3 and SeqID5 and SeqID6 as compared to conventional peptide-carrier vaccines (i.e., SeqID3+KLH and SeqID6+CRM197) a set of experiments was performed analysing the selectivity of the ensuing immune response elicited towards two different forms of the presynaptic protein aSyn: non aggregated, mainly monomeric aSyn as well as aggregated aSyn filaments.

### Vaccines Used:

| | T-cell | | | |
|---|---|---|---|---|
| B-cell | epitope/ | | | |
| epitope | carrier | CLEC | adjuvant | Route |
| SeqID2 | SeqID7 | Pustulan | n.a | i.d. |
| | | (80%) | | |
| SeqID3 | KLH | Pustulan | n.a | i.d. |
| | | (80%) | | |
| SeqID3 | KLH | n.a | Alhydrogel | s.c. |
| SeqID5 | SeqID7 | Pustulan | n.a | i.d. |
| | | (80%) | | |
| SeqID6 | CRM197 | Pustulan | n.a | i.d. |
| | | (80%) | | |
| SeqID6 | CRM197 | n.a | Alhydrogel | s.c. |

Animals (female Balb/c mice) were vaccinated 3 times in biweekly intervals (all vaccines: 20 μg of aSyn targeting peptide/dose; route: i.d. for the CLEC based vaccines and and s.c. for the KLH and CRM197 based vaccine adjuvanted with Alhydrogel) and the ensuing immune response against the target protein, i.e., recombinant human alpha synuclein as well as aSyn filament has been analysed using murine plasma taken two weeks after the third immunization. The plasma samples were subjected to an aSyn specific inhibition ELISA and IC50 values were determined.

### Results:

Briefly, all CLEC based conjugates used in this experiment demonstrate superior immunogenicity and aSynuclein aggregate specific target selectivity as compared to the conventional peptide-conjugate vaccines (i.e., SeqID3+KLH and SeqID6+CRM, see FIG. 12).

Conventional peptide conjugate vaccines can induce an antibody response with slightly increased selectivity for aSyn aggregates (i.e., filaments) as compared to monomeric/recombinant aSyn. SeqID3+KLH adjuvanted with Alhydrogel was mounting an immune response with 9-fold higher selectivity for aSyn aggregates as compared to recombinant aSyn. SeqID6+CRM197 adjuvanted with Alhydrogel was inducing a less selective immune response reaching 3.5-fold more selective binding directed towards aggregates as compared to mainly monomeric, recombinant aSyn.

In contrast, antibodies induced by CLEC based peptide conjugate vaccines were characterized by several fold more selective binding as compared to KLH or CRM197 conjugate vaccines. The SeqID2+SeqID7+pustulan and SeqID5+SeqID7+pustulan induced plasma shows an approx. 97-fold (i.e. 14× higher than the comparator vaccine SeqID3+KLH, Alhydrogel) and 50-fold higher aggregate selectivity (i.e. 14× higher than the comparator vaccine SeqID6+CRM, Alhydrogel). SeqID3+KLH+pustulan and SeqID6+CRM197+pustulan were similarly selective reaching 40- (i.e. 5 fold higher than SeqID3-KLH) and 50-fold (i.e. 14 times higher than SeqID6+CRM) higher selectivity for aSyn aggregates respectively.

Thus, the experiments show that CLEC modification of peptide conjugates as well as of peptide-protein conjugates leads to a strongly enhanced target specificity of the ensuing immune response providing a novel unprecedented strategy to optimize current state of the art conjugate vaccines.

### Example 13: Analysis of Avidity and Affinity of Immune Responses Elicited by CLEC Based Vaccines

To further characterize the immune responses elicited by CLEC based vaccines containing the aSyn targeting peptides SeqID2 and SeqID3 and SeqID5 and SeqID6 as compared to conventional peptide-carrier vaccines (i.e., SeqID3+KLH and SeqID6+CRM197) a set of experiments was performed analysing the avidity and affinity of the antibodies elicited towards aSyn.

### Vaccines Used:

| | T-cell | | | |
|---|---|---|---|---|
| B-cell | epitope/ | | | |
| epitope | carrier | CLEC | adjuvant | Route |
| SeqID2 | SeqID7 | Pustulan | n.a | i.d. |
| | | (80%) | | |
| SeqID3 | KLH | Pustulan | n.a | i.d. |
| | | (80%) | | |
| SeqID3 | KLH | n.a | Alhydrogel | s.c. |
| SeqID5 | SeqID7 | Pustulan | n.a | i.d. |
| | | (80%) | | |
| SeqID6 | CRM197 | Pustulan | n.a | i.d. |
| | | (80%) | | |
| SeqID6 | CRM197 | n. a | Alhydrogel | s.c. |

Animals (female Balb/c mice) were vaccinated 3 times in biweekly intervals (all vaccines: 20 μg of aSyn targeting peptide/dose; route: i.d. for the CLEC based vaccines and s.c. for the KLH and CRM197 based vaccine adjuvanted with Alhydrogel) and the ensuing immune response against the target protein, i.e., recombinant human aSyn as well as aSyn filament has been analysed using murine plasma taken two weeks after each immunization. To determine avidity of the induced Abs towards recombinant aSyn, a variation of the standard ELISA assay was used where replicate wells containing antibody bound to antigens were exposed to increasing concentrations of chaotropic thiocyanate ions. Resistance to thiocyanate elution was used as the measure of avidity and an index (avidity index) representing 50% of effective antibody binding was used to compare plasma samples (both between treatment groups and between time points).

In addition, the k_{D} value for aSyn filaments (antibody affinity toward aSyn filaments) of the antibodies 2 weeks after the last immunization was determined as well based on an aSyn competition ELISA.

### Results:

As shown in FIG. 13, conventional SeqID3+KLH conjugate (adjuvanted with Alhydrogel) showed only limited avidity maturation towards aSyn binding when comparing immune samples obtained two weeks after the second (T2) or two weeks after the third immunization (avidity maturation (AM, comparing IC₅₀ values for T2 and T3 samples: 1.1)). In contrast CLEC based vaccines like SeqID2+SeqID7+pustulan could induce a strong maturation of the anti-aSyn response as indicated by an AI of 2.2 associated with a strong increase in avidity of T3 samples towards aSynuclein. Samples obtained from animals undergoing SeqID3+KLH+pustulan immunisation also showed a significantly higher avidity and a slightly increased maturation as compared to SeqID3+KLH alone.

Similarly, avidity of the immune response elicited against aSyn proteins was also significantly higher for SeqID5+SeqID7+pustulan and SeqID6+CRM197+pustulan vaccine induced antibodies as compared to the SeqID6+CRM197 benchmark vaccine (analysed at T3; i.e. 3-3.8 times higher chaotropic salt levels were required to reduce binding) and affinity maturation was also increased comparing T2 and T3 values, respectively. SeqID6+CRM197 did not lead to an increase in avidity towards aSyn comparing T2 and T3 whereas the two CLEC based vaccines lead to a strong increase in aSyn specific binding comparing T2 and T3.

Experiments quantifying the aSyn filament k_{D} for the immune response elicited by CLEC based vaccines as well as conventional benchmark vaccines revealed a highly significant increase in the overall affinity of antibodies induced by CLEC based vaccines for aSyn (see FIG. 14). SeqID2+SeqID7+pustulan and SeqID3+KLH+pustulan conjugates showed a 6-9-fold higher affinity (i.e., Kd: 110 nM and 160 nM compared to a k_{D} of 1 mM) than the benchmark vaccine SeqID3+KLH adjuvanted with Alhydrogel. SeqID5+SeqID7+pustulan and SeqID6+CRM+pustulan conjugates are displaying 12-15 times better Kd values as the benchmark control SeqID6+CRM197, adjuvanted with Alhydrogel (i.e., Kd: 50 nM and 60 nM compared to a k_{D} of 750 nM).

The experiments therefore show that CLEC modification of peptide conjugates as well as of peptide-protein conjugates leads to a strongly enhanced target specificity and affinity of the ensuing immune response providing a novel unprecedented strategy to optimize current state of the art conjugate vaccines.

### Example 14: Analysis of In Vitro Functionality of Immune Responses Elicited by CLEC Based Vaccines

To analyse whether aSyn specific antibodies elicited by CLEC based vaccines (containing aSyn targeting peptides SeqID2/3 and SeqID5/6) are biologically active a set of experiments was performed analysing the capacity of antibodies to inhibit aSyn aggregation in vitro.

| | T-cell | | | |
|---|---|---|---|---|
| B-cell | epitope/ | | | |
| epitope | carrier | CLEC | adjuvant | Route |
| SeqId2 | SeqID7 | Pustulan | n.a | i.d. |
| | | (80%) | | |
| SeqID3 | KLH | n.a | Alhydrogel | s.c. |
| SeqID5 | SeqID7 | Pustulan | n.a | i.d. |
| | | (80%) | | |
| SeqID6 | CRM197 | Pustulan | n.a | i.d. |
| | | (80%) | | |
| SeqID6 | CRM197 | n.a | Alhydrogel | s.c. |

Animals (female Balb/c mice) were vaccinated 3 times in biweekly intervals (all vaccines: 20 μg of aSyn targeting peptide/dose; route: i.d. for the CLEC based vaccines and s.c. for the KLH and CRM197 based vaccine adjuvanted with Alhydrogel). Samples of murine plasma taken two weeks after each immunization as well as respective control samples (e.g.: non aSyn binding antibodies or pre-immune plasma obtained before immunization) have been analyzed for in vitro aggregation inhibition capacity.

### Results:

As shown in FIG. 15A, control antibodies or plasma taken from animals prior to immunization had no significant effects on the aggregation kinetics of aSyn confirming the specificity of the assay. Conventional SeqID3+KLH conjugate (adjuvanted with Alhydrogel) induced Abs were able to reduce aSyn aggregation significantly as indicated by a 40% decreased slope value (aSyn monomer only:100%; KLH. 60%). The SeqID2+SeqID7+pustulan vaccine induced Abs strongly inhibited aSyn aggregation as indicated by an 85% decreased slope value (aSyn monomer only:100%; CLEC:15%) in this assay indicating a significantly higher inhibition capacity as compared to classical vaccine induced Abs.

SeqID5+SeqID7+pustulan and SeqID6+CRM+pustulan based vaccine induced antibodies show 86-92% inhibition of the formation of aggregates starting with rec. aSyn (low content of aggregates) and 67-82% inhibition of the formation of aggregates starting with preformed fibrils (=bona fide aggregates) as compared to 68% and 57% for the benchmark vaccine SeqID6+CRM, Alhydrogel induced antibodies (see FIG. 15B).

### Example 15: Analysis of the Effects of the Route of Immunization on Immune Responses Elicited by CLEC Based Vaccines

A series of immunisations has been performed to compare i.d. administration to alternative routes including sub cutaneous (s.c.) and intra-muscular (i.m.).

### Vaccines Used:

| | T-cell | | | |
|---|---|---|---|---|
| B-cell | epitope/ | | | |
| epitope | carrier | CLEC | adjuvant | Route |
| SeqID2 | SeqID7 | Pustulan | n.a | i.d. |
| | | (80%) | | |
| SeqID2 | SeqID7 | Pustulan | n.a | s.c. |
| | | (80%) | | |
| SeqID2 | SeqID7 | Pustulan | n.a | i.m |
| | | (80%) | | |

Animals (female Balb/c mice) were vaccinated 3 times in biweekly intervals (all vaccines: 1 μg, 5 μg and 20 μg of aSyn targeting peptide/dose) and the ensuing immune response against the injected peptide and the target protein, i.e., recombinant human aSynuclein as well as aSyn filament has been analysed using murine plasma taken two weeks after the third immunization.

### Results:

Tables 1 and 2 and FIG. 16 show that SeqID2+SeqID7+pustulan vaccines applied via i.m. or s.c. routes could induce high immune responses against both, injected peptide (FIG. 16A) and anti-aSyn responses (FIG. 16B). Maximum titers reached were significantly lower than those following i.d. application at all doses tested. S.c. application showed a similar dose response behaviour as i.d. whereas i.m. did not show significant differences between 5 and 20 μg indicating a saturation at these doses/application volumes reached. Similar results were obtained for reactivity against monomeric as well as aggregated aSyn, respectively. These results demonstrate the high selectivity of the CLEC backbone as presented in this invention for application in skin as opposed to other routes/tissues.

TABLE
anti-SeqID2/3 induced antibody response following
WISIT vaccine application via different routes

| | 1 μg | 5 μg | 20 μg |
|---|---|---|---|
| i.d. | 16.000 | 83.000 | 140.000 |
| s.c. | 1000 | 2.000 | 12.000 |
| i.m. | 4.000 | 16.000 | 15.000 |

TABLE
anti-aSyn induced antibody response following
WISIT vaccine application via different routes

| | 1 μg | 5 μg | 20 μg |
|---|---|---|---|
| i.d. | 2.000 | 5.000 | 10.000 |
| s.c. | 100 | 1.000 | 4.000 |
| i.m. | 2.000 | 2.000 | 5.000 |

### Example 16: Analysis of Immunogenicity of CLEC Conjugates Using Carrier Proteins as T-Helper Cell Epitopes: Different Conjugate/CLEC Ratios

In this example immunogenicity of CLEC based conjugate vaccines containing the well-known carrier protein CRM197 using different peptide+CRM/CLEC ratios was compared. For this purpose, the aSyn derived epitope SeqID6 has been coupled to maleimide activated CRM197. Subsequently, SeqID6+CRM197 conjugate has been coupled to activated pustulan at different w/w ratios using the heterobifunctional linker BPMH to form CLEC based conjugate vaccines with CRM197 as source for T-helper cell epitopes to induce a sustainable immune response.

### Vaccines Used:

| | T-cell | | | | |
|---|---|---|---|---|---|
| B-cell | epitope/ | | Ratio | | |
| epitope | carrier | CLEC | (w/w) | adjuvant | Route |
| SeqID6 | CRM197 | Pustulan | 1/1 | n.a | i.d. |
| | | (80%) | | | |
| SeqID6 | CRM197 | Pustulan | 1/2.5 | n.a | i.d. |
| | | (80%) | | | |
| SeqID6 | CRM197 | Pustulan | 1/5 | n.a | i.d. |
| | | (80%) | | | |
| SeqID6 | CRM197 | Pustulan | 1/10 | n.a | i.d. |
| | | (80%) | | | |
| SeqID6 | CRM197 | Pustulan | 1/20 | n.a | i.d. |
| | | (80%) | | | |

Animals (female Balb/c mice) were vaccinated 3 times in biweekly intervals (all vaccines: 5 μg of aSyn targeting peptide/dose; route: i.d. for the CLEC based vaccines) and the ensuing immune response directed against the injected peptide (i.e., SeqID6) as well as against the target protein, i.e. recombinant human aSynuclein as well as aSyn filament has been analysed using murine plasma taken two weeks after the third immunization.

### Results:

As shown in FIG. 17, all 5 vaccines using CRM197 as source for T-helper epitopes were able to induce a strong and specific immune response against both, the injected peptide moieties (SeqID6) and the target protein: recombinant aSynuclein.

CLEC modification of the CRM197 conjugates led to a highly efficient immune response with all w/w Conjugate/CLEC ratios tested. SeqID6+CRM197+pustulan (w/w 1/10) was delivering highest anti-aSyn specific immune responses as compared to the other variants tested. Thus, SeqID6+CRM197 conjugates with medium/high Conjugate/CLEC ratios are especially suited for inducing optimal immune responses (e.g.: ⅕, 1/10 and 1/20).

Thus, the experiments show that CLEC modification of conventional peptide-protein conjugates leads to a strong target specificity of the ensuing immune response providing a novel unprecedented strategy to optimize current state of the art conjugate vaccines building on carrier proteins like KLH, CRM197 or others.

### Example 17: Analysis of Immunogenicity of CLEC Conjugates and Peptide Conjugates Using Carrier Proteins as T-Helper Cell Epitopes—aSyn N-Terminus (aa1-10)

In this example we assessed whether CLEC based conjugate vaccines according to the present invention are able to induce superior immune responses against aSyn aggregates as compared to respective peptide conjugates using state of the art carrier proteins as source for T-cell epitopes.

Therefore, a set of experiments was initiated comparing two conjugates, both containing an epitope suggested to be suitable as aSyn targeting epitope. Experiments could demonstrate the immune response elicited against injected peptide and aSyn protein as well as the selectivity of the ensuing immune response elicited towards two different forms of the presynaptic protein aSyn: non aggregated, mainly monomeric aSyn as well as aggregated aSyn filaments.

For example, Weihofen et al (2019, aa1-10 as epitope of Cinpanemab) and WO2016/062720 (aa1-8 as epitope in a VLP based immunotherapeutic) suggest the N-terminal aSyn sequence derived from position aa1-10 as a potentially suitable epitope for aSyn targeting immunotherapy. To assess whether CLEC modification indeed results in a superior immune response, we therefore compared a CLEC based vaccine containing aSyn sequence aa1-8 (SeqID12+SeqID7+pustulan) with the respective conventional peptide+KLH vaccine (SeqID13+KLH adjuvanted with Alum).

### Vaccines Used:

| | T-cell | | | |
|---|---|---|---|---|
| B-cell | epitope/ | | | |
| epitope | carrier | CLEC | adjuvant | Route |
| SeqID12 | SeqID7 | Pustulan | n.a | i.d. |
| | | (80%) | | |
| SeqID13 | KLH | na | Alum | i.d. |

Animals (female Balb/c mice) were vaccinated 3 times in biweekly intervals (all vaccines: 5 μg of aSyn targeting peptide/dose; route: i.d. for the CLEC based vaccines and s.c. for the KLH based vaccine adjuvanted with Alhydrogel) and the ensuing immune response against the injected peptide and the target protein by ELISA and EC50 values were determined. In addition, to assess selectivity of the immune response, the plasma samples were subjected to an aSyn specific inhibition ELISA and expressed as percentage of maximum binding.

### Results:

The aSyn N-terminus targeting CLEC based conjugate vaccine used in this experiment (SeqID12+SeqID7+Pus) demonstrates superior immunogenicity against aSyn protein as compared to the conventional peptide-conjugate vaccines (i.e., SeqID13+KLH see FIG. 18A). The CLEC based vaccine induces a 1.8-fold increase in anti aSyn titers and a concomitant 3-fold increase in the ratio of the anti-peptide to anti protein response as the comparator group. This strongly supports the teaching of this invention, that CLEC modification leads to a superior immune response as similar conventional vaccines.

In addition, the conventional peptide KLH conjugate vaccine induces an antibody response with strongly increased selectivity (ca. 10-fold) for aSyn monomers as compared to aggregates (i.e., filaments, see FIG. 18B). In contrast to this finding and very surprisingly, the CLEC based conjugate leads to a completely different selectivity: SeqID12+SeqID7+pustulan induces antibodies with a significantly, ca. 10-fold higher selectivity for aSyn aggregates as compared to recombinant aSyn thereby changing the profile of the antibodies induced completely (see FIG. 18B).

Thus, the experiments show that conventional peptide vaccines applying aSyn aa1-8 are less suitable to mount an efficient and selective immune response in vivo suggesting that this epitope is not suitable for aggregate selective immunotherapy. Importantly, the results also demonstrate that CLEC modification of peptide conjugates leads to a strongly enhanced target specificity of the ensuing immune response as well as a change in selectivity towards aggregates which is therefore providing a novel unprecedented conjugate vaccine targeting aSyn.

### Example 18: Analysis of Immunogenicity of CLEC Conjugates and Peptide Conjugates Using Carrier Proteins as T-Helper Cell Epitopes—aSyn aa100-108

Here, a set of experiments was initiated comparing two conjugates, both containing an epitope suggested to be suitable as aSyn targeting epitope by analysing the immune response elicited against injected peptide and aSyn protein as well as the selectivity of the ensuing immune response elicited towards two different forms of the presynaptic protein aSyn: non aggregated, mainly monomeric aSyn as well as aggregated aSyn filaments.

For example, WO 2011/020133 and WO2016/062720 suggest the aSyn sequence derived from position aa100-108/109 (either as native sequence or mimotope, i.e. 100-108) as a potentially suitable epitope for aSyn targeting immunotherapy. To assess whether CLEC modification indeed results in a superior immune response using this epitope region we therefore compared a CLEC based vaccine containing aSyn aa100-108 (SeqID16+SeqID7+pustulan) with the respective conventional peptide-KLH vaccine (SeqID17+KLH adjuvanted with Alum).

### Vaccines Used:

| B-cell | T-cell epitope/ | | | |
|---|---|---|---|---|
| epitope | carrier | CLEC | adjuvant | Route |
| SeqID16 | SeqID7 | Pustulan | n.a | i.d. |
| | | (80%) | | |
| SeqID17 | KLH | na | Alum | i.d. |

Animals (female Balb/c mice) were vaccinated 3 times in biweekly intervals (all vaccines: 5 μg of aSyn targeting peptide/dose; route: i.d. for the CLEC based vaccines and s.c. for the KLH based vaccine adjuvanted with Alhydrogel) and the ensuing immune response against the injected peptide and the target protein by ELISA and EC50 values were determined. In addition, to assess selectivity of the immune response, the plasma samples were subjected to an aSyn specific inhibition ELISA and expressed as percentage of maximum binding.

### Results:

The aSyn targeting CLEC based conjugate vaccine used in this experiment (SeqID16+SeqID7+Pus) demonstrates an overall very low anti aSyn protein response, also lower as compared to the conventional peptide-conjugate vaccines (i.e., SeqID17+KLH see FIG. 19A). The conventional vaccine induces an immune response characterized by a 2.1-fold increase in anti aSyn titers but at the same time a 2-fold decrease in the ratio of the anti-peptide/anti protein titers as the CLEC based vaccine. The latter finding supports the teaching of this invention, that CLEC modification leads to a superior anti target protein response, even in the case of overall lower immunogenicity as similar conventional vaccines.

In addition, both vaccines, the conventional peptide conjugate and the CLEC based vaccine, are less preferred to induce an aggregate selective immune response see FIG. 19B). Thus, the experiments provided show that CLEC-based and conventional peptide vaccines targeting the region aa100-108 are less suitable to mount an efficient and selective immune response in vivo suggesting that this epitope may not be the optimal choice for aggregate selective immunotherapy according to this invention.

### Example 19: Analysis of Immunogenicity of CLEC Conjugates and Peptide Conjugates Using Carrier Proteins as T-Helper Cell Epitopes—aSyn aa91-100

In this example, a set of experiments was initiated comparing two conjugates, both containing an epitope suggested to be suitable as aSyn targeting epitope by analysing the immune response elicited against injected peptide and aSyn protein.

For example, US 2014/0377271 suggests that the epitope aa91-99 acts as an autoepitope in PD patients and should therefore constitute a potentially suitable epitope for aSyn targeting immunotherapy. To assess whether CLEC modification indeed results in a superior immune response applying this epitope we therefore compared a CLEC based vaccine containing aSyn aa91-100 (SeqID14+SeqID7+pustulan) with the respective conventional peptide-KLH vaccine (SeqID15+KLH adjuvanted with Alum).

### Vaccines Used:

| B-cell | T-cell epitope/ | | | |
|---|---|---|---|---|
| epitope | carrier | CLEC | adjuvant | Route |
| SeqID14 | SeqID7 | Pustulan | n.a | i.d. |
| | | (80%) | | |
| SeqID15 | KLH | na | Alum | i.d. |

Animals (female Balb/c mice) were vaccinated 3 times in biweekly intervals (all vaccines: 5 μg of aSyn targeting peptide/dose; route: i.d. for the CLEC based vaccines and s.c. for the KLH based vaccine adjuvanted with Alhydrogel) and the ensuing immune response against the injected peptide and the target protein aSyn by ELISA and EC50 values were determined.

### Results:

Surprisingly, both vaccines induced relevant anti peptide titers but were less successful to induce detectable anti aSyn protein titers (see FIG. 20). Thus, the experiments provided show that CLEC-based and conventional peptide vaccines targeting the region aa91-100 are less suitable to mount an efficient and selective immune response in vivo suggesting that this epitope may not be the optimal choice for aggregate selective immunotherapy according to this invention.

### Example 20: Analysis of Immunogenicity of CLEC Conjugates and Peptide Conjugates Using Carrier Proteins as T-Helper Cell Epitopes—aSyn C-Terminal Region Aa131-140

In this example, a set of experiments was initiated comparing two conjugates, both containing an epitope suggested to be suitable as aSyn targeting epitope by analysing the immune response elicited against injected peptide and aSyn protein. In addition, the selectivity of the ensuing immune response elicited towards two different forms of the presynaptic protein aSyn was assessed.

For example, US 2015/0232524 and WO2016/062720 suggest the C-terminal aSyn sequence derived from positions aa-126-140 and 131-140 as potentially suitable epitope for aSyn targeting immunotherapy. To assess whether CLEC modification indeed results in a superior immune response we therefore compared a CLEC based vaccine containing aSyn aa131-140 (SeqID20+SeqID7+pustulan) with the respective conventional peptide-KLH vaccine (SeqID21+KLH adjuvanted with Alum).

### Vaccines Used:

| B-cell | T-cell epitope/ | | | |
|---|---|---|---|---|
| epitope | carrier | CLEC | adjuvant | Route |
| SeqID20 | SeqID7 | Pustulan | n.a | i.d. |
| | | (80%) | | |
| SeqID21 | KLH | na | Alum | i.d. |

Animals (female Balb/c mice) were vaccinated 3 times in biweekly intervals (all vaccines: 5 μg of aSyn targeting peptide/dose; route: i.d. for the CLEC based vaccines and s.c. for the KLH based vaccine adjuvanted with Alhydrogel) and the ensuing immune response against the injected peptide and the target protein by ELISA and EC50 values were determined. In addition, to assess selectivity of the immune response, the plasma samples were subjected to an aSyn specific inhibition ELISA and expressed as percentage of maximum binding.

### Results:

The aSyn targeting CLEC based conjugate vaccine used in this experiment (SeqID20+SeqID7+Pus) demonstrates an overall lower anti aSyn protein response as compared to the conventional peptide-conjugate vaccines (i.e., SeqID21+KLH see FIG. 21A). The conventional vaccine induces an immune response characterized by a 1.8-fold increase in anti aSyn titers but a 45% decrease in the ratio of the anti-peptide/anti protein titers as the CLEC based vaccine. The latter finding supports the teaching of this invention, that CLEC modification leads to a superior anti target protein response, even in the case of overall lower immunogenicity as similar conventional vaccines.

In addition, the conventional peptide conjugate are less suitable to induce an aggregate selective immune response (see FIG. 21B). In contrast, the CLEC based vaccine elicits antibodies with an approx. 10-fold increased selectivity for monomeric aSyn at the expense of aggregated aSyn (see FIG. 21B). Thus, the experiments provided show that CLEC-based and conventional peptide vaccines targeting the region aa131-140 are less preferred to mount an efficient and selective immune response towards aggregated aSyn in vivo suggesting that this epitope may not be the optimal choice for aggregate selective immunotherapy according to this invention.

### Example 21: Analysis of Immunogenicity of CLEC Conjugates and Peptide Conjugates Using Carrier Proteins as T-Helper Cell Epitopes—aSyn C-Terminal Region Aa103-135

In this example we assessed whether CLEC based conjugate vaccines according to the present invention can induce superior immune responses against aSyn as compared to respective peptide conjugates using state of the art carrier proteins as source for T-cell epitopes.

Therefore, a set of experiments was initiated comparing several conjugates, derived from an epitope region suggested to be suitable as aSyn targeting epitope. These experiments could demonstrate the immune response elicited against injected peptide and aSyn protein as well as the selectivity of the ensuing immune response elicited towards two different forms of the presynaptic protein aSyn: non aggregated, mainly monomeric aSyn as well as aggregated aSyn filaments.

Several studies suggest that the C-terminal aSyn sequence derived from position aa103-135 is a potentially suitable epitope for aSyn targeting immunotherapy either as source for autoepitopes, for peptides containing the original sequence or mimotopes thereof. To assess whether CLEC modification indeed results in a superior immune response using this region within aSyn we therefore compared several CLEC based vaccines (using peptides within region 107-126) with the respective conventional peptide-CRM vaccines (adjuvanted with Alum).

| B-cell | T-cell epitope/ | | | |
|---|---|---|---|---|
| epitope | carrier | CLEC | adjuvant | Route |
| SeqID56 | SeqID7 | Pustulan | n.a | i.d. |
| | | (80%) | | |
| SeqID58 | CRM | na | Alum | s.c. |
| SeqID53 | SeqID7 | Pustulan | n.a | i.d. |
| | | (80%) | | |
| SeqID55 | CRM | na | Alum | s.c. |
| SeqID51 | SeqID7 | Pustulan | n.a | i.d. |
| | | (80%) | | |
| SeqID52 | CRM | na | Alum | s.c. |
| SeqID65 | SeqID7 | Pustulan | n.a | i.d. |
| | | (80%) | | |
| SeqID66 | CRM | na | Alum | s.c. |
| SeqID67 | SeqID7 | Pustulan | n.a | i.d. |
| | | (80%) | | |
| SeqID68 | CRM | na | Alum | s.c. |
| SeqID69 | SeqID7 | Pustulan | n.a | i.d. |
| | | (80%) | | |
| SeqID70 | CRM | na | Alum | s.c. |
| SeqID71 | SeqID7 | Pustulan | n.a | i.d. |
| | | (80%) | | |
| SeqID72 | CRM | na | Alum | s.c. |

Animals (female Balb/c mice) were vaccinated 3 times in biweekly intervals (all vaccines: 5 μg of aSyn targeting peptide/dose; route: i.d. for the CLEC based vaccines and s.c. for the KLH based vaccine adjuvanted with Alhydrogel) and the ensuing immune response against the injected peptide and the target protein by ELISA and EC50 values were determined. In addition, to assess selectivity of the immune response, the plasma samples were subjected to an aSyn specific inhibition ELISA and expressed as percentage of maximum binding.

TABLE 1
immune response elicited by vaccines covering aa107-126

| | Peptide Titer | aSyn filament Titer |
|---|---|---|
| Vaccine | (x1000) | (x1000) |
| SeqDI56 + SeqID7 + Pus | 0.1 | 0.1 |
| SeqID58 + CRM + Alum | 9.781 | 0.1 |
| SeqDI53 + SeqID7 + Pus | 0.1 | 0.1 |
| SeqID55 + CRM + Alum | 17.647 | 0.8 |
| SeqDI51 + SeqID7 + Pus | 19.694 | 1.687 |
| SeqID52 + CRM + Alum | 13.115 | 1.594 |
| SeqDI65 + SeqID7 + Pus | 9.04 | 3.256 |
| SeqID66 + CRM + Alum | 7.984 | 1.108 |
| SeqDI67 + SeqID7 + Pus | 12.016 | 7.335 |
| SeqID68 + CRM + Alum | 18.126 | 1.864 |
| SeqDI69 + SeqID7 + Pus | 28.125 | 3.523 |
| SeqID70 + CRM + Alum | 37.88 | 1.054 |
| SeqDI71 + SeqID7 + Pus | 7.783 | 5.788 |
| SeqID72 + CRM + Alum | 14.603 | 2.429 |

CLEC based as well as CRMV based vaccines containing 5- and 6mer peptides both were less suitable to induce high anti aSyn filament titers in this experiment. The aSyn C-terminus targeting CLEC based conjugate vaccines (7- to 12-mer peptides) used in this experiment (see table 1 and FIGS. 22A, 23A, and 24A) all demonstrate superior immunogenicity against aSyn filaments as compared to the conventional peptide-conjugate vaccines (see table 1, up to 4-fold increase). This strongly supports the teaching of this invention, that CLEC modification leads to a superior immune response as similar conventional vaccines using epitopes derived from 103-135, especially 107-126.

Analysis of selectivity for aggregated aSyn further supports this teaching. As shown in FIGS. 22B and 23B, CLEC vaccines containing epitopes derived from sequence aa115-126 are surprisingly effective in eliciting highly aggregate selective immune responses. As shown in FIG. 22B, the CLEC based vaccine SeqID51+SeqID7+Pus, containing an 8-mer aSyn targeting epitope, induces antibodies with a 10-fold higher selectivity for aSyn aggregates whereas the respective conventional vaccine (SeqID52+CRMV+Alum) fails to induce aggregate selective antibodies. Similarly, CLEC based vaccine SeqID67+SeqID7+Pus containing a 10-mer aSyn targeting epitope, induces an approx. 10-fold higher selectivity for aSyn aggregates as compared to monomers whereas the respective conventional vaccine (SeqID68+CRM+Alum) elicits antibodies which are more selective, ca. 3-fold, for monomers as compared to aggregates (see FIG. 23B).

Analysis of selectivity for vaccines containing the epitope aa107-114 (SeqID73+SeqID7+Pus and SeqID74+CRM+Alum, see FIG. 24B) surprisingly showed that, despite the presence of high antia-Syn filament titers (i.e. superior immunogenicity) induced by the CLEC-based vaccine, neither CLEC- nor conventional vaccines could induce aggregate selective antibodies indicating that only highly selected peptide sequences within aa103-135 are suitable as immunotherapeutics targeting aggregated aSyn specifically.

It may also be noted, as shown before (see FIGS. 18-20), that epitopes derived from aa91-100, aa100-108 and aa131-140 are all less suitable as potential immunotherapeutic regions for targeting aggregated aSyn specifically.

### Example 22: Analysis of In Vitro Functionality of Immune Responses Elicited by CLEC Based Vaccines

To analyse whether aSyn specific antibodies elicited by CLEC based vaccines (containing aSyn targeting peptides from epitope region aa103-135) are biologically active, a set of experiments was performed analysing the capacity of antibodies to inhibit aSyn aggregation in vitro.

### Vaccines Used:

| B-cell | T-cell epitope/ | | | |
|---|---|---|---|---|
| epitope | carrier | CLEC | adjuvant | Route |
| SeqID67 | SeqID7 | Pustulan | n.a | i.d. |
| | | (80%) | | |
| SeqID68 | CRM | n.a | Alhydrogel | s.c. |
| SeqID71 | SeqID7 | Pustulan | n.a | i.d. |
| | | (80%) | | |
| SeqID72 | CRM197 | n.a | Alhydrogel | i.d. |
| SeqID73 | SeqID7 | Pustulan | n.a | i.d. |
| | | (80%) | | |
| SeqID74 | CRM197 | n.a | Alhydrogel | s.c. |

Animals (female Balb/c mice) were vaccinated 3 times in biweekly intervals (all vaccines: 20 μg of aSyn targeting peptide/dose; route: i.d. for the CLEC based vaccines and s.c. for the CRM197 based vaccines adjuvanted with Alhydrogel). Samples of murine plasma taken two weeks after each immunization as well as respective control samples (e.g.: the aSyn binding antibody LB509, epitope aa115-122, or pre-immune plasma obtained before immunization) have been analyzed for in vitro aggregation inhibition capacity.

### Results:

As shown in FIG. 25D, plasma taken from animals prior to immunization had no significant effects on the aggregation kinetics of aSyn confirming the specificity of the assay.

The SeqID67+SeqID7+pustulan vaccine (containing a 10-mer aSyn derived peptide) induced Abs strongly inhibited aSyn aggregation as indicated by an 40% decreased aggregation in this assay over time whereas the respective CRM conjugate vaccine was showing only minimal effects indicating a significantly higher inhibition capacity as compared to classical vaccine induced Abs (FIG. 25A). A similar result could be achieved analysing antibodies induced by SeqID71+SeqID7+pustulan (containing a 12-mer aSyn derived peptide), which could reduce aggregation even more strongly (70-80% inhibition) and could surpass inhibition capacity of antibodies induced by conventional CRM vaccine (SeqID72+CRM+Alhydrogel) by 2 to 2.5-fold. FIG. 25C shows that the antibodies induced by CLEC based and conventional vaccines building on epitope aa107-114 ((containing an 8-mer aSyn derived peptide) in contrast failed to inhibit aSyn aggregation.

As shown in FIG. 25D, the aSyn specific antibody LB509 fails to inhibit aSyn aggregation. In the contrary, a slight increase in aggregation can be detected in this analysis.

This is a highly surprising effect considering the teachings in this invention (see example 14 and FIG. 15 analysing epitopes derived of aSyn sequence aa115-126 as well, especially also aa115-121), as well as table 1 and FIGS. 22-25 describing vaccines covering epitopes 115-126) as the monoclonal LB509 (epitope aa:115-122) which is known to bind to different forms of aSyn (Jakes et al. Neurosci Lett. 1999 Jul. 2; 269(1):13-6) and shares the same epitope as biologically effective vaccines according to this invention (see FIG. 25D). It follows that the biologically superior effects obtained with CLEC based vaccines are indeed surprising and are showing that highly selected peptide sequences contained within aa115-126 are preferred within the region aa103-135 as immunotherapeutics targeting aggregated aSyn specifically.

### Example 23: Determination of Biological Activity of Peptide+CRM197+CLEC Conjugates In Vitro Towards Murine Dectin-1 Receptor

In a series of ELISA experiments conjugates containing the dectin-1 ligands pustulan, lichenan and laminarin have been assessed for their binding efficacy to murine dectin-1. Biological activity of the peptide+CRM197+CLEC conjugates is represented by their PRR binding ability. Along these lines and to ensure that the structure of CLEC (pustulan, lichenan, laminarin) remained biologically active after coupling, binding to murine dectin-1 was assessed. Non-oxidized and oxidized pustulan, lichenan and laminarin as well as CRM conjugate vaccine and peptide+CRM197+CLEC-based novel conjugates have then been assessed for their biological activity using a competitive ELISA system based on competitive binding of a soluble murine Fc-dectin-1a receptor (InvivoGen) as described in Korotchenko et al. 2020.

### Results:

Ensuing experiments revealed that the median molecular weight (20 kDa), linear β-(1,6) linked β-D-glucan pustulan and the linear β-(1-3)-glucan with β-(1-6)-linkages laminarin exert significantly higher binding efficacy (ca. 10-fold) to murine dectin-1 than the larger, high molecular weight, linear β-(1,3) β-(1,4)-β-D glucan lichenan (ca 245 kDa) (see FIG. 26).

As shown in FIG. 26A the dectin-1 ligand pustulan, oxidized pustulan, SeqID6+CRM conjugate (CRM-conjugate 1) and SeqID6+CRM+pustulan conjugate (CRM-Pus conjugate 1) have been assessed for their binding efficacy to murine dectin-1 by ELISA analysis. Ensuing experiments revealed that the peptide+CRM197+pustulan conjugate displays similar binding efficacy to murine dectin-1 as oxidized pustulan. In contrast, the conventional CRM-conjugate 1 displays no specific murine dectin-1 binding. High binding efficacy to murine dectin-1 is also shown as well by 5 novel CRM+pustulan conjugates (SeqID52/66/68/70/72) (FIG. 26B). Ensuing experiments revealed that peptide-CRM197-pustulan conjugates with different B-cell epitopes, ranging from 7-mer B-cell epitope (SeqID6+CRM+Pus; FIG. 26A) to 12-mer B-cell epitope (SeqID71+CRM+Pus; FIG. 1B) display similar binding efficacy to murine dectin-1 as oxidized pustulan. As shown in FIG. 26C, the high molecular weight (ca 22-245 kDa) linear, β-(1,3) β-(1,4)-β-D glucan lichenan, exerts lower binding efficacy, irrespective of oxidation or conjugation, than the linear β-(1,6) linked β-D-glucan pustulan based constructs. For example, pustulan containing CRM197-peptide conjugates retain an approx. 10-fold higher binding than lichenan based constructs. High binding efficacy to murine dectin-1 is also shown by the linear β-(1-3)-glucan with β-(1-6)-linkages laminarin (FIG. 26D). Ensuing experiments revealed that the peptide+CRM197+laminarin conjugate displays similar binding efficacy to murine dectin-1, irrespective of oxidation or conjugation, as for pustulan based constructs.

The experiment revealed that peptide+CRM197+CLEC conjugates demonstrate biological activity towards dendritic cells via binding to dectin-1 in the murine system.

### Example 24: Determination of Biological Activity of Peptide+CRM197+CLEC Conjugates In Vitro Towards Human Dectin-1 Receptor

In a series of ELISA experiments the dectin-1 ligands pustulan, lichenan and laminarin have been assessed for their binding efficacy to human dectin-1. Biological activity of the peptide+CRM197+CLEC conjugates is represented by their PRR binding ability. Along these lines and to ensure that the structure of CLEC (pustulan, lichenan, laminarin) remained biologically active after coupling, binding to human dectin-1 was assessed by competitive ELISA system based on competitive binding of a soluble human Fc-dectin-1a receptor (InvivoGen).

### Results:

As shown in FIG. 27, the SeqID6+CRM conjugate coupled to either lichenan (Lich conjugate), pustulan (Pus conjugate) or laminarin (Lam conjugate) have been assessed for their binding efficacy to human dectin-1 by ELISA analysis.

Ensuing experiments revealed that peptide+CRM197+pustulan vaccines exert significantly higher binding efficacy (ca. 30-fold) to human dectin-1 than vaccine conjugated to lichenan (see FIG. 27). In contrast, peptide+CRM197+laminarin vaccines display weak binding to human Dectin-1.

### Example 25: In Vivo Comparison of Different Peptide+CRM197+Pustulan-Based Vaccines

Novel CRM197-pustulan based vaccines with different B-cell epitopes, ranging from 8mer to 11mer, which were able to bind to their DC receptor (e.g.: dectin-1) were tested for their ability to induce a robust and specific immune response following repeated application in n=5 Balb/c mice/group. Typical experiments were performed applying 5 μg net peptide content of B-cell epitope peptides per dose.

In this experiment the aSyn derived peptide SeqID52+CRM197 and SeqID66/68/70+CRM conjugates, were coupled to oxidized pustulan. Animals (female Balb/c mice) were vaccinated 3 times in biweekly intervals (route: i.d.) with either β-glucan-modified or unmodified Peptide-CRM conjugates and the ensuing immune response directed against the injected peptides (i.e., SeqID52/66/68/70, respectively) and against aggregated aSyn filaments was analyzed using murine plasma taken two weeks after the third immunization.

| B-cell | T-cell epitope/ | | | |
|---|---|---|---|---|
| epitope | carrier | CLEC | adjuvant | Route |
| SeqID52 | CRM | Pustulan | n.a | i.d. |
| | | (80%) | | |
| SeqID66 | CRM | Pustulan | n.a | i.d. |
| | | (80%) | | |
| SeqID68 | CRM | Pustulan | n.a | i.d. |
| | | (80%) | | |
| SeqID70 | CRM | Pustulan | n.a | i.d. |
| | | (80%) | | |
| SeqID52 | CRM | n.a. | Alhydrogel | s.c. |
| SeqID66 | CRM | n.a | Alhydrogel | s.c. |
| SeqID68 | CRM | n.a | Alhydrogel | s.c. |
| SeqID70 | CRM | n.a. | Alhydrogel | s.c. |

### Vaccines Used:

As shown in FIG. 28A, all 4 CRM+pustulan based vaccines (SeqID52/66/68/70/72) were able to induce significantly higher responses against both, the injected peptide moieties (e.g.: SeqID52/66/68/70) and against aggregated aSyn filaments when compared to unmodified peptide+CRM based vaccines adjuvanted with Alhydrogel.

Peptide+CRM+pustulan based conjugates could induced 2-5× higher titers against the respective peptide (highest titers of 1/190.000) and 3-13× higher titers against aSyn filaments (highest titers of 1/29.000) as unmodified peptide+CRM based vaccines.

### Example 26: Analysis of Selectivity of Immune Responses Elicited by Peptide+CRM+Pustulan Based Vaccines In Vivo

To further characterize the immune responses elicited by peptide+CRM197+pustulan based vaccines containing different B-cell epitopes as compared to conventional peptide+CRM197 vaccines, a set of experiments was performed analysing the selectivity of the ensuing immune response elicited towards aggregated aSyn filaments.

Animals (female Balb/c mice) were vaccinated 3 times in biweekly intervals (all vaccines: 5 μg of aSyn targeting peptide/dose; route: i.d. for the 4 peptide+CRM197+CLEC based vaccines (SeqID52/SeqID66/68/70+CRM197+pus) and s.c. for the 4 peptide-CRM197 based vaccines (SeqID52/SeqID66/68/70-CRM197 adjuvanted with Alhydrogel) and the ensuing immune response against the target protein, i.e., recombinant human alpha Synuclein as well as aSyn filament has been analysed using murine plasma taken two weeks after the third immunization. The plasma samples were subjected to an aSyn specific inhibition ELISA and IC50 values were determined.

### Vaccines Used:

| B-cell | T-cell epitope/ | | | |
|---|---|---|---|---|
| epitope | carrier | CLEC | adjuvant | Route |
| SeqID52 | CRM | Pustulan | n.a | i.d. |
| | | (80%) | | |
| SeqID66 | CRM | Pustulan | n.a | i.d. |
| | | (80%) | | |
| SeqID68 | CRM | Pustulan | n.a | i.d. |
| | | (80%) | | |
| SeqID70 | CRM | Pustulan | n.a | i.d. |
| | | (80%) | | |
| SeqID52 | CRM | n.a. | Alhydrogel | s.c. |
| SeqID66 | CRM | n.a | Alhydrogel | s.c. |
| SeqID68 | CRM | n.a | Alhydrogel | s.c. |
| SeqID70 | CRM | n.a. | Alhydrogel | s.c. |

### Results:

Briefly, all CLEC based conjugates used in this experiment demonstrate superior aSyn aggregate specific target selectivity, as determined by a much lower IC50 value against aSyn filaments, as compared to the conventional peptide-CRM197 conjugate vaccines (see FIG. 29).

All 4 conventional peptide-CRM197 conjugate vaccines tested in this experiment induced antibodies demonstrating a very weak selectivity towards aSyn filaments, shown by very high IC50 values with 400-1.700 ng/ml.

In contrast, all antibodies induced by novel peptide+CRM197+pustulan based conjugate vaccines were characterized by much lower IC50 values for aSyn filaments ranging from 3.5-15 ng/ml.

Thus, the experiments show that CLEC modification of CRM197 conjugates leads to a strongly enhanced target specificity of the ensuing immune response, regardless of the epitope used, providing a novel unprecedented strategy to optimize current state of the art conjugate vaccines.

### Example 27: Analysis of Avidity of Immune Responses Elicited by Peptide+CRM197+Pustulan Based Vaccines

To further characterize the immune responses elicited by peptide-CRM197-pustulan based vaccines containing different B-cell epitopes as compared to conventional peptide-CRM197 vaccines a set of experiments was performed analysing the avidity of the antibodies elicited towards aSyn filaments.

Animals (female Balb/c mice) were vaccinated 3 times in biweekly intervals (all vaccines: 5 μg of aSyn targeting peptide/dose; route: i.d. for the CLEC based vaccines (SeqID52/66/68/70+CRM197+pustulan) and s.c. for CRM197 based vaccines adjuvanted with Alhydrogel (SeqID52/66/68/70+CRM197) and the ensuing immune response against the target protein, i.e., aSyn filament has been analysed using murine plasma taken two weeks after each immunization. To determine avidity of the induced Abs towards aSyn filaments, a variation of the standard ELISA assay was used where replicate wells containing antibody bound to antigens were exposed to increasing concentrations of chaotropic thiocyanate ions. Resistance to thiocyanate elution was used as the measure of avidity and an index (avidity index) representing 50% of effective antibody binding was used to compare plasma samples.

Vaccine used:

| B-cell | T-cell epitope/ | | | |
|---|---|---|---|---|
| epitope | carrier | CLEC | adjuvant | Route |
| SeqID52 | CRM197 | Pustulan | n.a | i.d. |
| | | (80%) | | |
| SeqID66 | CRM197 | Pustulan | n.a | i.d. |
| | | (80%) | | |
| SeqID68 | CRM197 | Pustulan | n.a | i.d. |
| | | (80%) | | |
| SeqID70 | CRM197 | Pustulan | n.a | i.d. |
| | | (80%) | | |
| SeqID52 | CRM197 | n.a. | Alhydrogel | s.c. |
| SeqID66 | CRM197 | n.a | Alhydrogel | s.c. |
| SeqID68 | CRM197 | n.a | Alhydrogel | s.c. |
| SeqID70 | CRM197 | n.a | Alhydrogel | s.c. |

### Results:

As shown in FIG. 30, all tested conventional peptide+CRM197 conjugate (adjuvanted with Alhydrogel) induced antibodies showed only limited binding strength towards aSyn filaments as demonstrated by very low avidity indexes ranging from 0.25 to 0.85. In contrast, all novel peptide+CRM197+pustulan based vaccines induced antibodies showed significantly higher binding strength towards aSyn filaments with AIs ranging from 0.5-2.2.

The experiments therefore show that CLEC modification of peptide-CRM197 conjugates leads to a strongly enhanced target specific immune response (titer), as well as to a strongly enhanced target specificity and affinity of the induced antibody response regardless of the epitope used, providing a novel unprecedented strategy to optimize current state of the art protein-conjugate vaccines, including CRM197.

### Example 28: In Vivo Comparison of Different Peptide+CRM197+CLEC-Based Vaccines

The aSyn derived peptide SeqID6+CRM197 conjugates coupled either to pustulan, lichenan, or laminarin were tested for their ability to induce a robust and specific immune response following repeated application in n=5 Balb/c mice/group. Typical experiments were performed applying 5 μg net peptide content of B-cell epitope peptides per dose. Animals (female Balb/c mice) were vaccinated 3 times in biweekly intervals (route: i.d.) and the ensuing immune response directed against the injected peptides (i.e., SeqID6) and against aggregated aSyn filaments is analyzed using murine plasma taken two weeks after the third immunization.

### Vaccines Used:

| B-cell | T-cell epitope/ | | | |
|---|---|---|---|---|
| epitope | carrier | CLEC | adjuvant | Route |
| SeqID6 | CRM197 | Pustulan | n.a | i.d. |
| | | (80%) | | |
| SeqID6 | CRM197 | Lichenan | n.a | i.d. |
| | | (200%) | | |
| SeqID6 | CRM197 | Laminarin | n.a | i.d. |
| | | (200%) | | |
| SeqID6 | CRM197 | n.a | Alhydrogel | s.c. |

### Results:

Vaccines tested could induce significant immune responses against the injected peptide (e.g. SeqID6) as well as against aggregated aSyn filaments following repeated immunization in mice. Peptide-CRM-pustulan based conjugates induced high titers against the respective peptide and high titers against aSyn filaments compared to conventional peptide-CRM-based vaccines and to peptide-CRM-based vaccines conjugated to laminarin or lichenan (see FIG. 31).

Specifically, SeqID6+CRM197+pustulan induced 1.6 fold higher titers directed against the injected peptide SeqID6 as compared to SeqID6+CRM197+lichenan and 12 fold higher titers as compared to SeqID6+CRM197+laminarin. SeqID6+CRM197+lichenan could induce 7.5 fold higher titers as compared to SeqID6+CRM197+laminarin, respectively.

Similarly, SeqID+CRM197+pustulan induces 3.1 fold higher titers directed against aSyn aggregates (filaments) as compared to SeqID6+CRM197+lichenan, 7.6 fold higher titers as compared to SeqID6+CRM197+laminarin and 6 fold higher titers as compared to non-CLEC modified SeqID6+CRM197 adjuvanted with Alum. SeqID6+CRM197+lichenan could induce 2.4 fold higher titers as compared to SeqID6+CRM197+laminarin and 2 fold higher titers as compared to non-CLEC modified SeqID6+CRM197 adjuvanted with Alum, respectively.

CLEC modification of peptide+CRM197 conjugates are providing a novel unprecedented strategy to optimize current state of the art protein-conjugate vaccines, including CRM197.

### Example 29: Determination of Biological Activity of Peptide+CLEC-Conjugates In Vitro Towards Murine and Human Dectin-1 Receptor

In a series of ELISA experiments the dectin-1 ligands pustulan, lichenan and laminarin have been assessed for their binding efficacy to murine and human dectin-1. Biological activity of the peptide-CLEC conjugates is represented by their PRR binding ability. Along these lines and to ensure that the structure of CLEC (pustulan, lichenan, laminarin) remained biologically active after coupling, binding to murine and human dectin-1 was assessed by competitive ELISA system based on competitive binding of a soluble murine and human Fc-dectin-1a receptor (InvivoGen).

### Results:

As shown in FIG. 32, the SeqID5+SeqID7+CLEC conjugates coupled to either lichenan (Lich conjugate), pustulan (Pus conjugate) or laminarin (Lam conjugate) have been assessed for their binding efficacy to murine and human dectin-1 by ELISA analysis.

Ensuing experiments revealed that peptide-pustulan vaccines exert significantly higher binding efficacy to murine and human dectin-1 than vaccines conjugated to lichenan (see FIG. 32A+B). In contrast, the peptide+laminarin vaccine display very high binding to murine dectin1 (FIG. 32A) but only exerts weak binding to human Dectin-1 (FIG. 32B).

### Example 30: In Vivo Comparison of Different Peptide+CLEC-Based Vaccines

CLEC based vaccines which were able to bind to murine and/or human dectin-1 were tested for their ability to induce a robust and specific immune response following repeated application in n=5 Balb/c mice/group. Typical experiments were performed applying 5 μg and 20 μg net peptide content of B-cell epitope peptides per dose.

In this experiment the aSynuclein derived peptide SeqID5 and the promiscuous T-helper cell epitope SeqID7 were coupled via C-terminal hydrazide linkers to oxidized pustulan, lichenan or laminarin.

### Vaccines Used:

| B-cell | T-cell | |
|---|---|---|
| epitope | epitope | CLEC |
| SeqID5 | SeqID7 | Pustulan |
| SeqID5 | SeqID7 | Lichenan |
| SeqID5 | SeqID7 | Laminarin |

Animals (female Balb/c mice) were vaccinated 3 times in biweekly intervals (dose: 5 μg (A) and 20 μg (B); route: i.d.) and the ensuing immune response directed against the injected peptides (i.e., SeqID6) was analyzed using murine plasma taken two weeks after the third immunization.

### Results:

As shown in FIG. 33, all three CLEC vaccines (SeqID5+SeqID7+pustulan, SeqID5+SeqID7+lichenan and SeqID5+SeqID7+laminarin were able to induce a detectable immune response. Interestingly, immunization using the vaccine based on laminarin did only induce a very low anti-peptide and anti aSyn response. In contrast, pustulan based conjugates could induce a significantly higher response. Lichenan based conjugates showed a lower immunogenicity as compared to pustulan-based conjugates but could induce higher titers as the laminarin based conjugate in this experiment.

This demonstrates that dectin-1 binding efficacy in vitro, especially also to human dectin-1, can be directly linked to in vivo immunogenicity and biological activity of the vaccines. This leads to the identification of pustulan or fragments thereof (i.e. linear β(1,6)-β-D glucans) as most efficacious glucan variant as proposed in this application.

### Example 31: Analysis of In Vivo Functionality of Immune Responses Elicited by CLEC Based Vaccines

To determine if aSyn specific antibodies elicited by CLEC based vaccines were able to inhibit aSyn fibril formation in vivo, a proof-of-concept experiment was initiated using an established seeding model for synucleinopathies [Sci. Adv. 2020, 6, eabc4364, doi:10.1126/sciadv.abc4364; DOI: 10.1126/sciadv.abc4364].

### Vaccines Used:

| B-cell | T-cell epitope/ | | | |
|---|---|---|---|---|
| epitope | carrier | CLEC | adjuvant | Route |
| SeqID5 | SeqID7 | Pustulan | n.a | i.d. |
| | | (80%) | | |
| na | na | Pustulan | n.a | i.d. |

In this model, C57BL/6 mice were stereotactically injected with a-syn pre-formed fibrils (PFFs) at the level of the right substantia nigra, subsequently causing widespread synucleinopathy, characterized specifically by phosphosynuclein immunopositive Lewy-like neurites and intracytoplasmic aggregates along anatomical connections. Animals were immunized four times at weeks 0, 2, 4 and 10 with SeqID5+SeqID7+pustulan vaccine, or non-conjugated CLEC as control, starting the first immunization on the day of PFF inoculation. 126 days post PFF injection, animals were sacrificed, and brains were analyzed for the presence of phospho-S129 aSyn-positive aggregates in selected brain areas including cerebral cortex, striatum, thalamus, substantia nigra and brainstem.

### Results:

Analysis of the ensuing immune response was performed using plasma and CSF obtained at the time of sacrifice. High antibody titers against the injected peptide were detected in plasma of SeqID5+SeqID7+pustulan vaccine treated animals. In contrast, no signal above background could be detected in the CLEC-only treated group (FIG. 34). Analysis of the anti-peptide titer in CSF also showed a high level of SeqID5+SeqID7+pustulan vaccine induced antibodies, whereas no signal above background was detectable for the vehicle treated animals (FIG. 34). Immunohistochemistry of brain sections showed high numbers of phospho-S129 aSyn-positive aggregates throughout all analyzed areas in the vehicle treated group indicating a strong propagation of aSyn pathology. In contrast, synucleinopathy was significantly reduced in SeqID5+SeqID7+pustulan vaccinated mice (FIG. 34). Of note, there was a strong and significant reciprocal correlation between the strength of the antibody response and the level of synucleinopathy in vaccine recipients (FIG. 34).

### Example 32: Analysis of Immunogenicity of Peptide+CRM+CLEC Conjugates

In this example carrier specific immunogenicity of CLEC based conjugate vaccines was compared to conventional carrier vaccines.

For this purpose, the alpha synuclein derived epitope SeqID6 has been coupled to maleimide activated CRM197. Subsequently, Peptide+CRM197 conjugates have been coupled to activated pustulan using the heterobifunctional linker BPMH to form CLEC based conjugate vaccines with CRM197 as source for T-helper cell epitopes to induce a sustainable immune response.

### Vaccines Used:

| B-cell | T-cell epitope/ | | | |
|---|---|---|---|---|
| epitope | carrier | CLEC | adjuvant | Route |
| SeqID6 | CRM197 | Pustulan | n.a | i.d. |
| | | (80%) | | |
| SeqID6 | CRM197 | n. a | Alhydrogel | s.c. |

Animals (female Balb/c mice) were vaccinated 3 times in biweekly intervals and the ensuing immune response directed against the carrier protein CRM197 has been analysed using murine plasma taken two weeks after the third immunization. Dose of SeqID6 containing vaccines: 20 μg and 100 μg of alpha synuclein targeting peptide/dose; route: i.d. for the CLEC based vaccines and s.c. for the CRM197 based vaccine adjuvanted with Alhydrogel (FIG. 35).

### Results:

Comparison of anti-carrier specific antibody responses revealed that conventional SeqID6+CRM197 based vaccines were able to dose dependently induce high anti-CRM197 titers. In contrast, CLEC based SeqID6+CRM197+pustulan vaccines used were inducing significantly lower anti CRM responses following repeated immunization using 20 μg and 100 μg doses (reduction: 4.5-5 fold; FIG. 35).

Thus, the experiments show that covalent CLEC modification of conventional peptide-protein conjugates impairs development of an anti-carrier response significantly, providing a novel unprecedented strategy to optimize current state of the art conjugate vaccines building on carrier proteins like KLH, CRM197 or others.

### Example 33: In Vivo Analysis of Anti-Pustulan/Glucan Immune Responses Following Immunisation

As already discussed in example 7, analysis of anti-CLEC antibodies induced by CLEC-based immunogens is important on two levels for the novelty and efficacy of the proposed CLEC-vaccines according to the present invention.

Along these lines, an extensive analysis of anti-pustulan antibodies in plasma samples of naïve and peptide+CRM+CLEC conjugate immunized Balb/c mice (n=5/group) prior to immunization and following repeated immunizations was performed.

### Vaccines Used:

| B-cell | T-cell | | | Ratio |
|---|---|---|---|---|
| epitope | epitope | CLEC | Adjuvant | (w/w) |
| n.a. | n.a. | Pustulan | n.a. | n.a. |
| SeqID6 | CRM197 | Pustulan | n.a | 1/1 |
| | | (80%) | | |
| SeqID6 | CRM197 | Pustulan | n.a | 1/2.5 |
| | | (80%) | | |
| SeqID6 | CRM197 | Pustulan | n.a | 1/5 |
| | | (80%) | | |
| SeqID6 | CRM197 | Pustulan | n.a | 1/10 |
| | | (80%) | | |
| SeqID6 | CRM197 | Pustulan | n.a | 1/20 |
| | | (80%) | | |
| SeqID6 | CRM197 | Lichenan | n.a | |
| | | (200%) | | |
| SeqID6 | CRM197 | Laminarin | n.a | |
| | | (200%) | | |

### Results:

Different types of samples were analysed in this example: FIG. 36A shows the anti-pustulan immunoreactivity from samples obtained from animals undergoing repeated SeqID6+CRM+pustulan, SeqID6+CRM+lichenan or SeqID6+CRM+laminarin immunizations (all vaccines: 20 μg of aSyn targeting peptide/dose). FIG. 36B shows the anti-pustulan immunoreactivity from samples obtained from animals undergoing repeated SeqID6+CRM+pustulan immunisations using vaccines containing different w/w peptide+CRM conjugate/CLEC ratios: i.e. conjugate/CLEC ratios of 1/1, 1/2.5, 1/5, 1/10 and 1/20 (all vaccines: 5 μg of aSyn targeting peptide/dose).

For control purposes samples obtained from animals prior to immunization as well as from non-oxidised CLEC treated animals were used.

As shown in FIG. 36, Balb/c animals analysed showed a preexisting low level immune response directed against glucans/pustulan/β(1,6)-β-D glucan.

All CLEC vaccines tested failed in significantly increasing pre-existing anti glucan responses or de novo inducing high immune responses directed against the glucan backbone in vivo. In contrast, repeated application of unconjugated, non-oxidized pustulan present in the control group led to the induction of a strong anti-glucan immune response by boosting antibody levels against pustulan >5 times (compared to pre-immune plasma). Non-CLEC modified peptide-CRM conjugates and lichenan- and laminarin-containing conjugates were unable to induce anti-pustulan titers above preimmune levels indicating specificity of the anti-glucan response detected.

In summary, these analyses could demonstrate that: despite presence of a low-level, pre-existing auto-reactivity against pustulan (IgG) in naïve Balb/c mice, no/very low vaccination dependent change of anti-pustulan immunoreactivity is detected following immunization using various CLEC conjugates. This is indicating a significant lowering of Glucan immunogenicity applying the novel vaccine design according to the present invention. This is in strong contrast to previously published results and therefore constitutes a surprising and inventive novel characteristic of the carbohydrate backbone (e.g. the β-glucans, especially the pustulan backbone) according to the present invention.

In addition, pre-existing anti-pustulan-responses do not seem to preclude immune reactions to the peptide component of WISIT vaccines as the injected peptide responses for all experiments revealed high anti-peptide titers.

### Example 34: In Vivo Comparison of the Effect of Glucan Conjugation on Immunogenicity of Peptide-Carrier Vaccines

To assess whether conjugation of CLECs to peptide-carrier immunogens is required for the induction of superior immunogenicity of the vaccines according to the present invention, a set of experiments was initiated comparing three vaccine preparations: a peptide-carrier conjugate modified covalently with β-glucan, a vaccine preparation containing a mix of the peptide-carrier conjugate and the β-glucan without conjugation and a non-modified, non-Alum adjuvanted peptide-carrier vaccine.

Again, n=5 female Balb/c mice were immunized i.d. three times in biweekly intervals and the ensuing immune response directed against the injected peptide and aSyn filament (i.e., SeqID6) was analyzed using murine plasma taken two weeks after the third immunization.

### Vaccines Used:

| B-cell | | | |
|---|---|---|---|
| epitope | Carrier | CLEC | CLEC-Conjugation |
| SeqID6 | CRM197 | Pustulan (80%) | yes |
| SeqID6 | CRM197 | Pustulan (non-ox.) | No; mixing only |
| SeqID6 | CRM197 | n.a., no adjuvant | no |

### Results:

FIG. 37 shows the comparison of anti-peptide (SeqID6) and anti aSyn monomer specific immune responses detectable following three immunizations. SeqID6+CRM197+pustulan conjugates were able to induce approx. 10 times higher immune responses against the injected peptide (FIG. 37A) and approx. 4-fold higher anti aSyn titers (FIG. 37B) as reported for the mix of SeqID6+CRM197 and non-oxidized pustulan as well as approx. 10 fold higher aSyn titers as those for SeqID6+CRM197 (w/o adjuvant) in this experiment. Interestingly, mixing of SeqID6+CRM197 and non-oxidized pustulan did not result in a significantly different immune response as conventional SeqID6+CRM197.

These data show that conjugation of peptide+carrier immunogens to activated CLECs according to the current invention is required to induce a superior immune response in vivo.

B-cell epitope sequences disclosed in the examples were as follows:

| | | Sequence |
|---|---|---|
| SeqID | Sequence | Position |
| SeqID1 | (H2N-NH-CO-CH2-CH2-CO)- | aSyn, aa115-121 |
| | DQPVLPD | |
| SeqID2 | DQPVLPD-(NH-NH2) | aSyn, aa115-121 |
| SeqID3 | C-DQPVLPD | aSyn, aa115-121 |
| SeqID4 | (H2N-NH-CO-CH2-CH2-CO)- | aSyn, aa115-121 |
| | DMPVDPD | |
| SeqID5 | DMPVDPD-(NH-NH2) | aSyn, aa115-121 |
| SeqID6 | C-DMPVDPD | aSyn, aa115-121 |
| SeqID10 | DAEFRH-(NH-NH2) | Aβ, aa1-6 |
| SeqID11 | DAEFRH-C | Aβ, aa1-6 |
| SeqID12 | MDVFMKGL-(NH-NH2) | aSyn, aa1-8 |
| SeqID13 | MDVFMKGL-C | aSyn, aa1-8 |
| SeqID14 | ATGFVKKDQL-(NH-NH2) | aSyn, aa91-100 |
| SeqID15 | ATGFVKKDQL-C | aSyn, aa91-100 |
| SeqID16 | LGKNEEGAP-(NH-NH2) | aSyn, aa100-108 |
| SeqID20 | EGYQDYEPEA-(NH-NH2) | aSyn, aa131-140 |
| SeqID21 | EGYQDYEPEA-C | aSyn, aa131-140 |
| SeqID51 | DMPVDPDN-NH-NH2 | aSyn, aa115-122 |
| SeqID52 | CDMPVDPDN | aSyn, aa115-122 |
| SeqID53 | DMPVDP-(NH-NH2) | aSyn, aa115-120 |
| SeqID55 | C-DMPVDP | aSyn, aa115-120 |
| SeqID56 | DMPVD-(NH-NH2) | aSyn, aa115-119 |
| SeqID58 | C-DMPVD | aSyn, aa115-119 |
| SeqID65 | DMPVDPDNE-(NH-NH2) | aSyn, aa115-123 |
| SeqID66 | DMPVDPDNE-C | aSyn, aa115-123 |
| SeqID67 | DMPVDPDNEA-(NH-NH2) | aSyn, aa115-124 |
| SeqID68 | DMPVDPDNEA-C | aSyn, aa115-124 |
| SeqID69 | DMPVDPDNEAY-(NH-NH2) | aSyn, aa115-125 |
| SeqID70 | DMPVDPDNEAY-C | aSyn, aa115-125 |
| SeqID71 | DMPVDPDNEAYE-(NH-NH2) | aSyn, aa115-126 |
| SeqID72 | DMPVDPDNEAYE-C | aSyn, aa115-126 |
| SeqID73 | APQEGILE-(NH-NH2) | aSyn, aa107-114 |
| SeqID74 | APQEGILE-C | aSyn, aa107-114 |

Based on this general disclosure of the present invention and these examples, the following preferred embodiments of the present invention are disclosed:
1. A conjugate consisting of or comprising at least a β-glucan or a mannan and at least a B-cell or T-cell epitope polypeptide, wherein the β-glucan or mannan is covalently conjugated to the B-cell and/or T-cell epitope polypeptide to form a conjugate of the β-glucan or mannan and the B-cell and/or T-cell epitope polypeptide, wherein the B-cell and/or a T-cell epitope polypeptide is an alpha synuclein polypeptide; or
a conjugate consisting of or comprising at least a β-glucan or a mannan and at least an alpha synuclein B-cell epitope polypeptide, wherein the β-glucan or mannan is covalently conjugated to the B-cell epitope polypeptide to form a conjugate of the β-glucan or mannan and the B-cell epitope polypeptide.
2. A conjugate according to embodiment 1, wherein the β-glucan is a predominantly linear β-(1,6)-glucan with a ratio of (1,6)-coupled monosaccharide moieties to non-β-(1,6)-coupled monosaccharide moieties of at least 1:1, preferably at least 2:1, more preferred, at least 5:1, especially at least 10:1.
3. A conjugate according to embodiment 1 or 2, wherein the β-glucan is a dectin-1 binding β-glucan, preferably pustulan, lichenan, laminarin, curdlan, β-glucan peptide (BGP), schizophyllan, scleroglucan, whole glucan particles (WGP), zymosan, or lentinan, more preferred pustulan, laminarin, lichenan, lentinan, schizophyllan, or scleroglucan, especially pustulan; and/or wherein the β-glucan is a strong dectin-1 binding β-glucan, preferably
   a β-glucan which binds to the soluble murine Fc-dectin-1a receptor with an IC50 value lower than 10 mg/ml, more preferred with an IC50 value lower than 1 mg/ml, even more preferred with an IC50 value lower than 500 μg/ml, especially with an IC50 value lower than 200 μg/ml, as determined by a competitive ELISA; and/or
   wherein the conjugates bind to the soluble murine Fc-dectin-1a receptor with an IC50 value lower than 1 mg/ml, more preferred with an IC50 value lower than 500 μg/ml, even more preferred with an IC50 value lower than 200 μg/ml, especially with an IC50 value lower than 100 μg/ml, as determined by a competitive ELISA; and/or
   a β-glucan which binds to the soluble human Fc-dectin-1a receptor with an IC50 value lower than 10 mg/ml, more preferred with an IC50 value lower than 1 mg/ml, even more preferred with an IC50 value lower than 500 μg/ml, especially with an IC50 value lower than 200 μg/ml, as determined by a competitive ELISA; and/or
   wherein the conjugates bind to the soluble human Fc-dectin-1a receptor with an IC50 value lower than 1 mg/ml, more preferred with an IC50 value lower than 500 μg/ml, even more preferred with an IC50 value lower than 200 μg/ml, especially with an IC50 value lower than 100 μg/ml, as determined by a competitive ELISA.
4. A conjugate according to any of the embodiments 1 to 3, wherein the polypeptides comprise at least one B-cell epitope of alpha synuclein and at least one T-cell epitope, preferably a B-cell epitope+CRM197 conjugate covalently linked to β-glucan, especially a peptide+CRM197+linear β-(1,6)-glucan or a peptide+CRM197+linear pustulan conjugate.
5. A conjugate according to any one of embodiments 1 to 4, wherein the ratio of β-glucan or mannan to B-cell and/or T-cell epitope polypeptide in the conjugate, especially pustulan to peptide ratios, is from 10:1 (w/w) to 0.1:1 (w/w), preferably from 8:1 (w/w) to 2:1 (w/w), especially 4:1 (w/w), with the proviso if the conjugate comprises a carrier protein, the preferred ratio of β-glucan or mannan to B-cell-epitope+carrier polypeptide is from 50:1 (w/w), to 0.1:1 (w/w), especially 10:1 to 0.1:1.
6. A conjugate according to any one of embodiments 1 to 5, wherein a B-cell epitope and a pan-specific/promiscuous T-cell epitope is independently coupled to the β-glucan.
7. A conjugate according to any one of embodiments 1 to 6, wherein the B-cell epitope polypeptide has a length of 5 to 20 amino acid residues, preferably of 6 to 19 amino acid residues, especially of 7 to 15 amino acid residues; and/or wherein the T-cell epitope polypeptide has a length of 8 to 30 amino acid residues, preferably of 13 to 29 amino acid residues, especially of 13 to 28 amino acid residues,
wherein the B-cell epitope and/or the T-cell epitope is preferably linked to the β-glucan or mannan and/or to a carrier protein by a linker, more preferred a cysteine residue or a linker comprising a cysteine or glycine residue, a linker resulting from hydrazide-mediated coupling, from coupling via heterobifunctional linkers, such as N-β-maleimidopropionic acid hydrazide (BMPH), 4-[4-N-maleimidophenyl]butyric acid hydrazide (MPBH), N-[ε-Maleimidocaproic acid) hydrazide (EMCH) or N-[κ-maleimidoundecanoic acid]hydrazide (KMUH), from imidazole mediated coupling, from reductive amination, from carbodiimide coupling a —NH—NH₂ linker; an NRRA, NRRA-C or NRRA-NH—NH₂ linker, peptidic linkers, such as bi-, tri-, tetra- (or longer)-meric peptide groups, such as CG or CG, or cleavage sites, such as a cathepsin cleavage site; or combinations thereof, especially by a cysteine or NRRA-NH—NH₂ linker;
wherein the T-cell epitope is preferably a polypeptide comprising the amino acid sequence AKFVAAWTLKAAA, optionally linked to a linker, such as a cysteine residue or a linker comprising a cysteine residue, an NRRA, NRRA-C or NRRA-NH—NH2 linker; or a variant of the amino acid sequence AKFVAAWTLKAAA, wherein the variants include the amino acid sequence AKFVAAWTLKAA, variants wherein the first residue alanine is replaced by an aliphatic amino acid residue, such as glycine, valine, isoleucine and leucine, variants wherein the third residue phenylalanine is replaced with L-cyclohexylalanine, variants wherein the thirteenth amino acid residue alanine is replaced by an aliphatic amino acid residue (e.g. glycine, valine, isoleucine and leucine), variants comprising aminocaproic acid, preferably coupled to the C-terminus of the amino acid sequence AKFVAAWTLKAA, variants with the amino acid sequence AX₁FVAAX₂TLX₃AX₄A, wherein X₁ is selected from the group consisting of W, F, Y, H, D, E, N, Q, I, and K; X₂ is selected from the group consisting of F, N, Y, and W, X₃ is selected from the group consisting of H and K, and X₄ is selected from the group consisting of A, D, and E, with the proviso that the oligopeptide sequence is not AKFVAAWTLKAAA; especially wherein the T-cell epitope is selected from AKFVAAWTLKAAANRRA-(NH—NH2), AKFVAAWTLKAAAN-C, AKFVAAWTLKAAA-C, AKFVAAWTLKAAANRRA-C, aKXVAAWTLKAAaZC, aKXVAAWTLKAAaZCNRRA, aKXVAAWTLKAAa, aKXVAAWTLKAAaNRRA, aA (X) AAAKTAAAAa, aA (X) AAATLKAAa, aA (X) VAAATLKAAa, aA (X) IAAATLKAAa, aK (X) VAAWTLKAAa, and aKFVAAWTLKAAa, wherein X is L-cyclohexylalanine, Z is aminocaproic acid and a is an aliphatic amino acid residue selected from alanine, glycine, valine, isoleucine and leucine; and/or wherein the T-cell epitope is an alpha synuclein polypeptide selected from the group GKTKEGVLYVGSKTK (aa31-45), KTKEGVLYVGSKTKE (aa32-46), EQVTNVGGAVVTGVT (aa61-75), VTGVTAVAQKTVEGAGNIAAATGFVK (aa71-86), DPDNEAYEMPSE (aa116-130), DNEAYEMPSEEGYQD (aa121-135), and EMPSEEGYQDYEPEA (aa126-140).
8. A conjugate according to any one of embodiments 1 to 7, wherein the conjugate further comprises a carrier protein, preferably non-toxic cross-reactive material of diphtheria toxin (CRM), especially CRM₁₉₇, KLH, diphtheria toxoid (DT), tetanus toxoid (TT), Haemophilus influenzae protein D (HipD), and the outer membrane protein complex of serogroup B meningococcus (OMPC), recombinant non-toxic form of Pseudomonas aeruginosa exotoxin A (rEPA), flagellin, Escherichia coli heat labile enterotoxin (LT), cholera toxin (CT), mutant toxins (e.g., LTK63 and LTR72), virus-like particles, albumin binding protein, bovine serum albumin, ovalbumin, a synthetic peptide dendrimer e.g. a Multiple antigenic peptide (MAP), especially wherein the ratio of carrier protein to β-glucan or mannan in the conjugate is from 1/0.1 to 1/50, preferably 1/0.1 to 1/40, more preferred from 1/0.1 to 1/20, especially from 1/0.1 to 1/10,
wherein preferably the conjugate consists or comprises
(a) a β-glucan or a mannan
(b) an alpha synuclein polypeptide as the at least one B-cell or T-cell epitope polypeptide, and
(c) a carrier protein,
wherein the three components (a), (b) and (c) are covalently conjugated with each other in the sequence (a)-(b)-(c), (a)-(c)-(b) or (b)-(a)-(c), especially in the sequence (a)-(c)-(b); and
wherein preferably all these components (a), (b) and (c) are conjugated by linkers.
9. A conjugate according to any one of embodiments 1 to 8, wherein the polypeptide is or comprises at least one alpha synuclein B-cell or a T-cell epitope polypeptide and wherein the conjugate further comprises a non-alpha synuclein B-cell or T-cell epitope
10. A conjugate according to any one of embodiments 1 to 8, wherein the alpha-synuclein polypeptide is native alpha synuclein or a polypeptide comprising or consisting of amino acid residues 1 to 5, 1 to 8, 1 to 10, 60 to 100, 70 to 140, 85 to 99, 91 to 100, 100 to 108, 102 to 108, 102 to 109, 103 to 129, 103 to 135, 107 to 130, 109 to 126, 110 to 130, 111 to 121, 111 to 135, 115 to 121, 115 to 122, 115 to 123, 115 to 124, 115 to 125, 115 to 126, 118 to 126, 121 to 127, 121 to 140, or 126 to 135, of the amino acid sequence of native human alpha synuclein: MDVFMKGLSK AKEGVVAAAE KTKQGVAEAA GKTKEGVLYV GSKTKEGVVH GVATVAEKTK EQVTNVGGAV VTGVTAVAQK TVEGAGSIAA ATGFVKKDQL GKNEEGAPQE GILEDMPVDP DNEAYEMPSE EGYQDYEPEA (human aSyn (1-140 aa): UNIPROT accession number P37840),
preferably a polypeptide comprising or consisting of amino acid residues 1 to 8, 91 to 100, 100 to 108, 103 to 135, 107 to 130, 110 to 130, 115 to 121, 115 to 122, 115 to 123, 115 to 124, 115 to 125, 115 to 126, 118 to 126, 121 to 127, or 121 to 140; or mimotopes selected from the group DQPVLPD, DQPVLPDN, DQPVLPDNE, DQPVLPDNEA, DQPVLPDNEAY, DQPVLPDNEAYE, DSPVLPDG, DHPVHPDS, DTPVLPDS, DAPVTPDT, DAPVRPDS, and YDRPVQPDR.
10. A conjugate according to any one of embodiments 1 to 9, wherein the conjugate comprises a T-cell epitope and is free of B-cell epitopes, wherein the conjugate preferably comprises more than one T-cell epitope, especially two, three, four or five T-cell epitopes.
11. A conjugate according to any one of embodiments 1 to 10 for use as active vaccine for the treatment and prevention of synucleopathies, preferably Parkinson's disease (PD), dementia with Lewy bodies (DLB), multiple system atrophy (MSA), Parkinson's disease dementia (PDD), neuroaxonal dystrophies, Alzheimer's Disease with Amygdalar Restricted Lewy Bodies (AD/ALB).
12. A conjugate according to any one of embodiments 1 to 11 for use to induce target-specific immune responses while inducing no or only very limited CLEC- or carrier protein-specific antibody responses; and/or
for the induction of target specific immune responses while inducing no or only very limited CLEC- or carrier-protein specific antibody responses; and/or
for use in diseases with reduced or dysfunctional Treg populations to augment waning/reduced Treg number and activity and thereby reduce autoimmune reactivity of disease specific T-effector cells and dampen autoimmune responses in patients, wherein T-cell epitopes suitable as Treg epitopes, or a combination with Treg inducing agents, such as rapamycin, low-dose IL-2, TNF receptor 2 (TNFR2) agonist, anti-CD20 antibodies (such as rituximab), prednisolone, inosine pranobex, glatiramer acetate, or sodium butyrate; are used; and/or
for use in a treatment for augmenting or preserving T-cell numbers, especially T-effector cell numbers, and T-cell function in a PD patient, which preferably includes a combination of checkpoint inhibitors or vaccines using anti-immune check point inhibitor epitopes to induce an anti-immune checkpoint inhibitor immune response to augment or preserve T-cell numbers, especially T-effector cell numbers and T-cell function in a PD patient, wherein the PD patient is preferably selected by having an overall reduction of CD3+ cells, especially of CD3+CD4+ cells typical for PD patients at all stages of the disease; preferably a patient in H+Y stages 1-4, more preferred H+Y1-3, most preferred H+Y 2-3.
13. A conjugate according to any one of embodiments 1 to 12, wherein the β-glucan or mannan is for use as C-type lectin (CLEC) polysaccharide adjuvant, preferably for enhancing the T-cell response to a given T-cell epitope polypeptide, more preferred wherein the T-cell epitope is a linear T-cell epitope, especially wherein the T-cell epitope is a polypeptide comprising or consisting of the amino acid sequences SeqID7, 8, 22-29, 87-131, GKTKEGVLYVGSKTK, KTKEGVLYVGSKTKE, EQVTNVGGAVVTGVT, VTGVTAVAQKTVEGAGNIAAATGFVK, MPVDPDNEAYEMPSE), DNEAYEMPSEEGYQD, EMPSEEGYQDYEPEA, or combinations thereof.
14. A conjugate according to any one of embodiments 1 to 13 for use in increasing affinity maturation with respect to a specific alpha synuclein polypeptide antigen or for inducing an increased immune response with respect to the human self-antigen alpha synuclein.
15. A conjugate according to any one of embodiments 1 to 14 further comprising a carrier protein comprising T-cell epitopes for use in reducing or eliminating the B-cell response to the CLEC and/or to the carrier protein and/or in enhancing the T-cell response to the T-cell epitopes of the carrier protein, preferably wherein the carrier protein is non-toxic cross-reactive material of diphtheria toxin (CRM)), especially CRM197, KLH, diphtheria toxoid (DT), tetanus toxoid (TT), Haemophilus influenzae protein D (HipD), and the outer membrane protein complex of serogroup B meningococcus (OMPC), recombinant non-toxic form of Pseudomonas aeruginosa exotoxin A (rEPA), flagellin, Escherichia coli heat labile enterotoxin (LT), cholera toxin (CT), mutant toxins (e.g., LTK63 and LTR72), virus-like particles, albumin binding protein, bovine serum albumin, ovalbumin, a synthetic peptide dendrimer e.g. a Multiple antigenic peptide (MAP), especially wherein the T-cell epitope efficacy in a vaccine comprising linear T-cell epitopes is augmented, e.g. by an N- or C-terminal addition of a lysosomal protease cleavage site, such as a Cathepsin L-like cleavage site or an Cathepsin S-like cleavage site, wherein the Cathepsin L-like cleavage site is preferably defined by the following consensus sequence:

Xₙ—X₁—X₂—X₃—X₄—X₅—X₆—X₇—X₈
Xₙ: 3-27 amino acids from the immunogenic peptide
X₁: any amino acid
X₂: any amino acid
X₃: any amino acid
X₄: N/D/A/Q/S/R/G/L; preferred N/D, more preferred N
X₅: F/R/A/K/T/S/E; preferred F or R, more preferred R
X₆: F/R/A/K/V/S/Y; preferred F or R, more preferred R
X₇: any amino acid, preferred A/G/P/F, more preferred A
X₈: cysteine or Linker like NHNH₂,
wherein the most preferred sequence is Xₙ-X₁X₂X₃NRRA-Linker; and wherein the Cathepsin S-like cleavage site is preferably defined by the following consensus sequence:

Xₙ—X₁—X₂—X₃—X₄—X₅—X₆—X₇—X₈
Xₙ: 3-27 amino acids from the immunogenic peptide
X₁: any amino acid
X₂: any amino acid
X₃: any amino acid, preferred V,L,I,F,W,Y,H, more preferred V
X₄: any amino acid, preferred V,L,I,F,W,Y,H, more preferred V
X₅: K,R, E, D, Q, N, preferably K, R more preferably R
X₆: any amino acid
X₇: any amino acid, preferred A
X₈: preferred A
X₈: cysteine or linker like NHNH₂, wherein the most preferred sequence is Xₙ-X₁X₂VVRAA-Linker
16. A method for producing a conjugate according to any one of embodiments 1 to 15, wherein the β-glucan or mannan is activated by oxidation and wherein the activated β-glucan or mannan is contacted with the B-cell and/or the T-cell epitope polypeptide, thereby obtaining a conjugate of the β-glucan or mannan with the B-cell and/or the T-cell epitope polypeptide.
17. A method according to embodiment 16, wherein the β-glucan or mannan is obtained by periodate oxidation at vicinal hydroxyl groups, as reductive amination, or as cyanylation of hydroxyl groups.
18. A method according to embodiment 16 or 17, wherein the β-glucan or mannan is oxidized to an oxidation degree defined as the reactivity with Schiff's fuchsin-reagent corresponding to an oxidation degree of an equal amount of pustulan oxidized with periodate at a molar ratio of 0.2-2.6 preferably of 0.6-1.4, especially 0.7-1.
19. A method according to any one of embodiments 16 to 18, wherein the conjugate is produced by hydrazone based coupling for conjugating hydrazides to carbonyls (aldehyde) or coupling by using hetero-bifunctional, maleimide-and-hydrazide linkers (e.g.: BMPH (N-β-maleimidopropionic acid hydrazide, MPBH (4-[4-N-maleimidophenyl]butyric acid hydrazide), EMCH (N-[ε-Maleimidocaproic acid) hydrazide) or KMUH (N-[κ-maleimidoundecanoic acid]hydrazide) for conjugating sulfhydryls (e.g.: cysteines) to carbonyls (aldehyde).
20. A vaccine product designed for vaccinating an individual against a specific antigen, wherein the product comprises a compound comprising a β-glucan or mannan as a C-type lectin (CLEC) polysaccharide adjuvant covalently coupled to the specific antigen.
21. Vaccine product according to embodiment 20, wherein the product comprises a conjugate according to any one of embodiments 1 to 16 or obtainable or obtained by a method according to any one of embodiments 16 to 19.
22. Vaccine product according to embodiment 20 or 21, wherein the antigen comprises at least one B-cell epitope and at least one T-cell epitope, preferably wherein the antigen is a polypeptide comprising one or more B-cell and T-cell epitopes.
23. Vaccine product according to any one of embodiments 20 to 22, wherein the covalently coupled antigen and CLEC polysaccharide adjuvant are present as particles with a size of 1 to 5000 nm, preferably of 1 to 200 nm, especially of 2 to 160 nm, determined as hydrodynamic radius (HDR) by dynamic light scattering (DLS).
24. Vaccine product according to any one of embodiments 20 to 23, wherein the covalently coupled antigen and CLEC polysaccharide adjuvant are present as particles with a size of 1 to 50 nm, preferably of 1 to 25 nm, especially of 2 to 15 nm, determined as HDR by DLS.
25. Vaccine product according to any one of embodiments 20 to 24, wherein the covalently coupled antigen and CLEC polysaccharide adjuvant are present as particles with a size smaller than 100 nm, preferably smaller than 70 nm, especially smaller than 50 nm, determined as HDR by DLS.
26. Pharmaceutical composition comprising a conjugate or vaccine as defined in any one of embodiments 1 to 25 and a pharmaceutically acceptable carrier.
27. Pharmaceutical composition according to embodiment 26, wherein the pharmaceutically acceptable carrier is a buffer, preferably a phosphate or TRIS based buffer.
28. Pharmaceutical composition according to embodiment 26 or 27 contained in a needle-based delivery system, preferably a syringe, a mini-needle system, a hollow needle system, a solid microneedle system, or a system comprising needle adaptors; an ampoule, needle-free injection systems, preferably a jet injector; a patch, a transdermal patch, a microstructured transdermal system, a microneedle array patch (MAP), preferably a solid MAP (S-MAP), coated MAP (C-MAP) or dissolving MAP (D-MAP); an electrophoresis system, a iontophoresis system, a laser-based system, especially an Erbium YAG laser system; or a gene gun system.
29. Pharmaceutical composition according to any one of embodiments 26 to 28, wherein the conjugate or vaccine in contained as a solution or suspension, deep-frozen solution or suspension; lyophilizate, powder, or granulate.

1. A conjugate consisting of or comprising at least a β-glucan or a mannan and at least a B-cell or T-cell epitope polypeptide, wherein the β-glucan or mannan is covalently conjugated to the B-cell and/or T-cell epitope polypeptide to form a conjugate of the β-glucan or mannan and the B-cell and/or T-cell epitope polypeptide and wherein the B-cell and/or a T-cell epitope polypeptide is an alpha synuclein polypeptide.

2. A conjugate according to claim 1, wherein the β-glucan is a dectin-1 binding β-glucan, preferably pustulan, lichenan, laminarin, curdlan, β-glucan peptide (BGP), schizophyllan, scleroglucan, whole glucan particles (WGP), zymosan, or lentinan, more preferred pustulan, laminarin, lichenan, lentinan, schizophyllan, or scleroglucan, especially wherein the β-glucan is pustulan; and/or wherein the β-glucan a strong dectin-1 binding β-glucan, preferably a β-glucan which binds to the soluble murine Fc-dectin-1a receptor with an IC50 value lower than 10 mg/ml, more preferred with an IC50 value lower than 1 mg/ml, even more preferred with an IC50 value lower than 500 μg/ml, especially with an IC50 value lower than 200 μg/ml, as determined by a competitive ELISA; and/or wherein the conjugates bind to the soluble murine Fc-dectin-1a receptor with an IC50 value lower than 1 mg/ml, more preferred with an IC50 value lower than 500 μg/ml, even more preferred with an IC50 value lower than 200 μg/ml, especially with an IC50 value lower than 100 μg/ml, as determined by a competitive ELISA.

3. A conjugate according to claim 1, wherein the β-glucan is a predominantly linear P-(1,6)-glucan with a ratio of (1,6)-coupled monosaccharide moieties to non-β-(1,6)-coupled monosaccharide moieties of at least 1:1, preferably at least 2:1, more preferred, at least 5:1, especially at least 10:1.

4. A conjugate according to claim 1, wherein the alpha synuclein polypeptides comprise at least one B-cell epitope.

5. A conjugate according to claim 1, wherein a B-cell epitope and a pan-specific/promiscuous T-cell epitope is independently coupled to the β-glucan or mannan.

6. A conjugate according to claim 1, wherein the B-cell epitope polypeptide has a length of 5 to 20 amino acid residues, preferably of 6 to 19 amino acid residues, especially of 7 to 15 amino acid residues.

7. A conjugate according to claim 1, wherein the T-cell epitope polypeptide has a length of 8 to 30 amino acid residues, preferably of 13 to 29 amino acid residues, especially of 13 to 28 amino acid residues.

8. A conjugate according to claim 1, wherein the conjugate further comprises a carrier protein, preferably non-toxic cross-reactive material of diphtheria toxin (CRM), especially CRM₁₉₇, KLH, diphtheria toxoid (DT), tetanus toxoid (TT), Haemophilus influenzae protein D (HipD), and the outer membrane protein complex of serogroup B meningococcus (OMPC), recombinant non-toxic form of Pseudomonas aeruginosa exotoxin A (rEPA), flagellin, Escherichia coli heat labile enterotoxin (LT), cholera toxin (CT), mutant toxins (e.g., LTK63 and LTR72), virus-like particles, albumin binding protein, bovine serum albumin, ovalbumin, a synthetic peptide dendrimer e.g. a Multiple antigenic peptide (MAP).

9. A conjugate according to claim 1, wherein the polypeptide is or comprises a B-cell or a T-cell epitope polypeptide, preferably wherein the polypeptide is or comprises a B-cell and a T-cell epitope.

10. A conjugate according to claim 1, wherein the conjugate comprises a T-cell epitope, preferably a T-cell epitope comprising the amino acid sequence AKFVAAWTLKAAA, optionally linked to a linker, preferably AKFVAAWTLKAAANRRA-(NH—NH2), AKFVAAWTLKAAAN-C, AKFVAAWTLKAAAC, AKFVAAWTLKAAANRRA-C; or a variant thereof selected from aKXVAAWTLKAAaZC, aKXVAAWTLKAAaZCNRRA, aKXVAAWTLKAAa, aKXVAAWTLKAAaNRRA, aA(X)AAAKTAAAAa, aA(X)AAATLKAAa, aA(X)VAAATLKAAa, aA(X)IAAATLKAAa, aK(X)VAAWTLKAAa, and aKFVAAWTLKAAa, wherein X is L-cyclohexylalanine, Z is aminocaproic acid and a is an aliphatic amino acid residue selected from alanine, glycine, valine, iso-leucine and leucine.

11. A conjugate according to claim 1, wherein the ratio of β-glucan or mannan to B-cell and/or T-cell epitope polypeptide in the conjugate is from 10:1 (w/w) to 0.1:1 (w/w), preferably from 8:1 (w/w) to 2:1 (w/w), especially 4:1 (w/w).

12. An active anti-alpha synuclein vaccine for the treatment and prevention of synucleopathies, preferably Parkinson's disease (PD), dementia with Lewy bodies (DLB), multiple system atrophy (MSA), Parkinson's disease dementia (PDD), neuroaxonal dystrophies, Alzheimer's Disease with Amygdalar Restricted Lewy Bodies (AD/ALB) comprising a conjugate according to claim 1.

13. A method for producing a conjugate according claim 1, wherein the β-glucan or mannan is activated by oxidation and wherein the activated β-glucan or mannan is contacted with the B-cell and/or the T-cell epitope polypeptide, thereby obtaining a conjugate of the β-glucan or mannan with the B-cell and/or the T-cell epitope polypeptide.

14. A method according to claim 13, wherein the β-glucan or mannan is obtained by periodate oxidation at vicinal hydroxyl groups, as reductive amination, or as cyanylation of hydroxyl groups.

15. A method according to claim 13, wherein the β-glucan or mannan is oxidized to an oxidation degree defined as the reactivity with Schiff's fuchsin-reagent corresponding to an oxidation degree of an equal amount of pustulan oxidized with periodate at a molar ratio of 0.2-2.6 preferably of 0.6-1,4, especially 0.7-1.


SEQUENCE LISTING (567 sequences)

SEQ ID NO: 1 - protein, 7 aa, synthetic construct
  REGION 1..7: note = peptide
DQPVLPD

SEQ ID NO: 2 - protein, 7 aa, synthetic construct
  REGION 1..7: note = peptide
DQPVLPD

SEQ ID NO: 3 - protein, 8 aa, synthetic construct
  REGION 1..8: note = peptide
CDQPVLPD

SEQ ID NO: 4 - protein, 7 aa, synthetic construct
  REGION 1..7: note = peptide
DMPVDPD

SEQ ID NO: 5 - protein, 7 aa, synthetic construct
  REGION 1..7: note = peptide
DMPVDPD

SEQ ID NO: 6 - protein, 8 aa, synthetic construct
  REGION 1..8: note = peptide
CDMPVDPD

SEQ ID NO: 7 - protein, 17 aa, synthetic construct
  REGION 1..17: note = peptide
AKFVAAWTLKAAANRRA

SEQ ID NO: 8 - protein, 15 aa, synthetic construct
  REGION 1..15: note = peptide
AKFVAAWTLKAAANC

SEQ ID NO: 9 - protein, 6 aa, synthetic construct
  REGION 1..6: note = peptide
DAEFRH

SEQ ID NO: 10 - protein, 6 aa, synthetic construct
  REGION 1..6: note = peptide
DAEFRH

SEQ ID NO: 11 - protein, 7 aa, synthetic construct
  REGION 1..7: note = peptide
DAEFRHC

SEQ ID NO: 12 - protein, 8 aa, synthetic construct
  REGION 1..8: note = peptide
MDVFMKGL

SEQ ID NO: 13 - protein, 9 aa, synthetic construct
  REGION 1..9: note = peptide
MDVFMKGLC

SEQ ID NO: 14 - protein, 10 aa, synthetic construct
  REGION 1..10: note = peptide
ATGFVKKDQL

SEQ ID NO: 15 - protein, 11 aa, synthetic construct
  REGION 1..11: note = peptide
ATGFVKKDQLC

SEQ ID NO: 16 - protein, 9 aa, synthetic construct
  REGION 1..9: note = peptide
LGKNEEGAP

SEQ ID NO: 17-19 - skipped (3 entries)

SEQ ID NO: 20 - protein, 10 aa, synthetic construct
  REGION 1..10: note = peptide
EGYQDYEPEA

SEQ ID NO: 21 - protein, 11 aa, synthetic construct
  REGION 1..11: note = peptide
EGYQDYEPEAC

SEQ ID NO: 22 - protein, 13 aa, synthetic construct
  REGION 1..13: note = peptide
AKFVAAWTLKAAA

SEQ ID NO: 23 - skipped

SEQ ID NO: 24 - protein, 23 aa, synthetic construct
  REGION 1..23: note = peptide
DSETADNLEKTVAALSILPGHGC

SEQ ID NO: 25 - protein, 27 aa, synthetic construct
  REGION 1..27: note = peptide
DSETADNLEKTVAALSILPGHGCNRRA

SEQ ID NO: 26 - protein, 19 aa, synthetic construct
  REGION 1..19: note = peptide
ISITEIKGVIVHRIETILF

SEQ ID NO: 27 - protein, 23 aa, synthetic construct
  REGION 1..23: note = peptide
ISITEIKGVIVHRIETILFNRRA

SEQ ID NO: 28 - protein, 17 aa, synthetic construct
  REGION 1..17: note = peptide
ISQAVHAAHAEINEAGR

SEQ ID NO: 29 - protein, 21 aa, synthetic construct
  REGION 1..21: note = peptide
ISQAVHAAHAEINEAGRNRRA

SEQ ID NO: 30 - protein, 8 aa, synthetic construct
  REGION 1..8: note = peptide
DQPVLPDC

SEQ ID NO: 31-50 - skipped (20 entries)

SEQ ID NO: 51 - protein, 8 aa, synthetic construct
  REGION 1..8: note = peptide
DMPVDPDN

SEQ ID NO: 52 - protein, 9 aa, synthetic construct
  REGION 1..9: note = peptide
CDMPVDPDN

SEQ ID NO: 53 - protein, 6 aa, synthetic construct
  REGION 1..6: note = peptide
DMPVDP

SEQ ID NO: 54 - protein, 6 aa, synthetic construct
  REGION 1..6: note = peptide
DMPVDP

SEQ ID NO: 55 - protein, 7 aa, synthetic construct
  REGION 1..7: note = peptide
CDMPVDP

SEQ ID NO: 56 - protein, 5 aa, synthetic construct
  REGION 1..5: note = peptide
DMPVD

SEQ ID NO: 57 - protein, 5 aa, synthetic construct
  REGION 1..5: note = peptide
DMPVD

SEQ ID NO: 58 - protein, 6 aa, synthetic construct
  REGION 1..6: note = peptide
CDMPVD

SEQ ID NO: 59-64 - skipped (6 entries)

SEQ ID NO: 65 - protein, 9 aa, synthetic construct
  REGION 1..9: note = peptide
DMPVDPDNE

SEQ ID NO: 66 - protein, 10 aa, synthetic construct
  REGION 1..10: note = peptide
DMPVDPDNEC

SEQ ID NO: 67 - protein, 10 aa, synthetic construct
  REGION 1..10: note = peptide
DMPVDPDNEA

SEQ ID NO: 68 - protein, 11 aa, synthetic construct
  REGION 1..11: note = peptide
DMPVDPDNEAC

SEQ ID NO: 69 - protein, 11 aa, synthetic construct
  REGION 1..11: note = peptide
DMPVDPDNEAY

SEQ ID NO: 70 - protein, 12 aa, synthetic construct
  REGION 1..12: note = peptide
DMPVDPDNEAYC

SEQ ID NO: 71 - protein, 12 aa, synthetic construct
  REGION 1..12: note = peptide
DMPVDPDNEAYE

SEQ ID NO: 72 - protein, 13 aa, synthetic construct
  REGION 1..13: note = peptide
DMPVDPDNEAYEC

SEQ ID NO: 73 - protein, 8 aa, synthetic construct
  REGION 1..8: note = peptide
APQEGILE

SEQ ID NO: 74 - protein, 9 aa, synthetic construct
  REGION 1..9: note = peptide
APQEGILEC

SEQ ID NO: 75-86 - skipped (12 entries)

SEQ ID NO: 87 - protein, 14 aa, synthetic construct
  REGION 1..14: note = peptide
AKFVAAWTLKAAAC

SEQ ID NO: 88 - protein, 18 aa, synthetic construct
  REGION 1..18: note = peptide
AKFVAAWTLKAAANRRAC

SEQ ID NO: 89 - protein, 15 aa, synthetic construct
  REGION 1..15: note = peptide
  VARIANT 1: note = aliphatic amino acid residue selected from alanine, glycine, valine, isoleucine and leucine
  VARIANT 3: note = L cyclohexylalanine
  VARIANT 14: note = aminocaproic acid
  VARIANT 13: note = aliphatic amino acid residue selected from alanine, glycine, valine, isoleucine and leucine
XKXVAAWTLKAAXXC

SEQ ID NO: 90 - protein, 19 aa, synthetic construct
  REGION 1..19: note = peptide
  VARIANT 1: note = aliphatic amino acid residue selected from alanine, glycine, valine, isoleucine and leucine
  VARIANT 3: note = L cyclohexylalanine
  VARIANT 14: note = aminocaproic acid
  VARIANT 13: note = aliphatic amino acid residue selected from alanine, glycine, valine, isoleucine and leucine
XKXVAAWTLKAAXXCNRRA

SEQ ID NO: 91 - protein, 13 aa, synthetic construct
  REGION 1..13: note = peptide
  VARIANT 1: note = aliphatic amino acid residue selected from alanine, glycine, valine, isoleucine and leucine
  VARIANT 3: note = L cyclohexylalanine
  VARIANT 13: note = aliphatic amino acid residue selected from alanine, glycine, valine, isoleucine and leucine
XKXVAAWTLKAAX

SEQ ID NO: 92 - protein, 17 aa, synthetic construct
  REGION 1..17: note = peptide
  VARIANT 1: note = aliphatic amino acid residue selected from alanine, glycine, valine, isoleucine and leucine
  VARIANT 3: note = L cyclohexylalanine
  VARIANT 13: note = aliphatic amino acid residue selected from alanine, glycine, valine, isoleucine and leucine
XKXVAAWTLKAAXNRRA

SEQ ID NO: 93 - protein, 20 aa, synthetic construct
  REGION 1..20: note = peptide
DSETADNLEKTTAALSILPG

SEQ ID NO: 94 - protein, 24 aa, synthetic construct
  REGION 1..24: note = peptide
DSETADNLEKTTAALSILPGNRRA

SEQ ID NO: 95 - protein, 15 aa, synthetic construct
  REGION 1..15: note = peptide
LSEIKGVIVHRLEGV

SEQ ID NO: 96 - protein, 19 aa, synthetic construct
  REGION 1..19: note = peptide
LSEIKGVIVHRLEGVNRRA

SEQ ID NO: 97 - protein, 18 aa, synthetic construct
  REGION 1..18: note = peptide
KLLSLIKGVIVHRLEGVE

SEQ ID NO: 98 - protein, 22 aa, synthetic construct
  REGION 1..22: note = peptide
KLLSLIKGVIVHRLEGVENRRA

SEQ ID NO: 99 - protein, 15 aa, synthetic construct
  REGION 1..15: note = peptide
VSIDKFRIFCKANPK

SEQ ID NO: 100 - protein, 16 aa, synthetic construct
  REGION 1..16: note = peptide
LKFIIKRYTPNNEIDS

SEQ ID NO: 101 - protein, 15 aa, synthetic construct
  REGION 1..15: note = peptide
IREDNNTLKLDRCNN

SEQ ID NO: 102 - protein, 21 aa, synthetic construct
  REGION 1..21: note = peptide
FNNFTVSFWLRVPKVSASHLE

SEQ ID NO: 103 - protein, 14 aa, synthetic construct
  REGION 1..14: note = peptide
QYIKANSKFIGITE

SEQ ID NO: 104 - protein, 20 aa, synthetic construct
  REGION 1..20: note = peptide
LEYIPEITLPVIAALSIAES

SEQ ID NO: 105 - protein, 20 aa, synthetic construct
  REGION 1..20: note = peptide
LINSTKIYSYFPSVISKVNQ

SEQ ID NO: 106 - protein, 20 aa, synthetic construct
  REGION 1..20: note = peptide
NYSLDKIIVDYNLQSKITLP

SEQ ID NO: 107 - protein, 20 aa, synthetic construct
  REGION 1..20: note = peptide
PHHTALRQAILCWGELMTLA

SEQ ID NO: 108 - protein, 15 aa, synthetic construct
  REGION 1..15: note = peptide
FFLLTRILTIPQSLD

SEQ ID NO: 109 - protein, 16 aa, synthetic construct
  REGION 1..16: note = peptide
YSGPLKAEIAQRLEDV

SEQ ID NO: 110 - protein, 14 aa, synthetic construct
  REGION 1..14: note = peptide
FFLLTRILTIPQSL

SEQ ID NO: 111 - protein, 24 aa, synthetic construct
  REGION 1..24: note = peptide
GAYARCPNGTRALTVAELRGNAEL

SEQ ID NO: 112 - protein, 25 aa, synthetic construct
  REGION 1..25: note = peptide
ALNIWDRFDVFCTLGATTGYLKGNS

SEQ ID NO: 113 - protein, 15 aa, synthetic construct
  REGION 1..15: note = peptide
QYIKANSKFIGITEL

SEQ ID NO: 114 - protein, 21 aa, synthetic construct
  REGION 1..21: note = peptide
FNNFTVSFWLRVPKVSASHLE

SEQ ID NO: 115 - protein, 16 aa, synthetic construct
  REGION 1..16: note = peptide
KFIIKRYTPNNEIDSF

SEQ ID NO: 116 - protein, 16 aa, synthetic construct
  REGION 1..16: note = peptide
VSIDKFRIFCKALNPK

SEQ ID NO: 117 - protein, 17 aa, synthetic construct
  REGION 1..17: note = peptide
WVRDIIDDFTNESSQKT

SEQ ID NO: 118 - protein, 19 aa, synthetic construct
  REGION 1..19: note = peptide
AGLTLSLLVICSYLFISRG

SEQ ID NO: 119 - protein, 20 aa, synthetic construct
  REGION 1..20: note = peptide
PGPLRESIVCYFMVFLQTHI

SEQ ID NO: 120 - protein, 18 aa, synthetic construct
  REGION 1..18: note = peptide
VPGLYSPCRAFFNKEELL

SEQ ID NO: 121 - protein, 15 aa, synthetic construct
  REGION 1..15: note = peptide
TGHGARTSTEPTTDY

SEQ ID NO: 122 - protein, 13 aa, synthetic construct
  REGION 1..13: note = peptide
KELKRQYEKKLRQ

SEQ ID NO: 123 - protein, 11 aa, synthetic construct
  REGION 1..11: note = peptide
TVFYNIPPMPL

SEQ ID NO: 124 - protein, 14 aa, synthetic construct
  REGION 1..14: note = peptide
DKREMWMACIKELH

SEQ ID NO: 125 - protein, 11 aa, synthetic construct
  REGION 1..11: note = peptide
FVFTLTVPSER

SEQ ID NO: 126 - protein, 13 aa, synthetic construct
  REGION 1..13: note = peptide
PKYVKQNTLKLAT

SEQ ID NO: 127 - protein, 16 aa, synthetic construct
  REGION 1..16: note = peptide
EKKIAKMEKASSVFNV

SEQ ID NO: 128 - protein, 10 aa, synthetic construct
  REGION 1..10: note = peptide
FFLLTRILTI

SEQ ID NO: 129 - protein, 19 aa, synthetic construct
  REGION 1..19: note = peptide
DQSIGDLIAEAMDKVGNEG

SEQ ID NO: 130 - protein, 14 aa, synthetic construct
  REGION 1..14: note = peptide
QVHFQPLPPAVVKL

SEQ ID NO: 131 - protein, 16 aa, synthetic construct
  REGION 1..16: note = peptide
KQIINMWQEVGKAMYA

SEQ ID NO: 132 - protein, 20 aa, synthetic construct
  REGION 1..20: note = peptide
APDTRPAPGSTAPPAHGVTS

SEQ ID NO: 133 - protein, 19 aa, synthetic construct
  REGION 1..19: note = peptide
GVTSAPDTRPAPGSTPPAH

SEQ ID NO: 134-234 - skipped (101 entries)

SEQ ID NO: 235 - protein, 18 aa, synthetic construct
  REGION 1..18: note = peptide
KLLSLIKGVIVHRLEGVE

SEQ ID NO: 236-302 - skipped (67 entries)

SEQ ID NO: 303 - protein, 20 aa, synthetic construct
  REGION 1..20: note = peptide
NYSLDKIIVDYNLQSKITLP

SEQ ID NO: 304 - protein, 20 aa, synthetic construct
  REGION 1..20: note = peptide
LINSTKIYSYFPSVISKVNQ

SEQ ID NO: 305 - protein, 20 aa, synthetic construct
  REGION 1..20: note = peptide
LEYIPEITLPVIAALSIAES

SEQ ID NO: 306-333 - skipped (28 entries)

SEQ ID NO: 334 - protein, 7 aa, synthetic construct
  REGION 1..7: note = peptide
DQPVLPD

SEQ ID NO: 335 - protein, 8 aa, synthetic construct
  REGION 1..8: note = peptide
DQPVLPDN

SEQ ID NO: 336 - protein, 9 aa, synthetic construct
  REGION 1..9: note = peptide
DQPVLPDNE

SEQ ID NO: 337 - protein, 10 aa, synthetic construct
  REGION 1..10: note = peptide
DQPVLPDNEA

SEQ ID NO: 338 - protein, 11 aa, synthetic construct
  REGION 1..11: note = peptide
DQPVLPDNEAY

SEQ ID NO: 339 - protein, 12 aa, synthetic construct
  REGION 1..12: note = peptide
DQPVLPDNEAYE

SEQ ID NO: 340 - protein, 8 aa, synthetic construct
  REGION 1..8: note = peptide
DSPVLPDG

SEQ ID NO: 341 - protein, 8 aa, synthetic construct
  REGION 1..8: note = peptide
DHPVHPDS

SEQ ID NO: 342 - protein, 8 aa, synthetic construct
  REGION 1..8: note = peptide
DTPVLPDS

SEQ ID NO: 343 - protein, 8 aa, synthetic construct
  REGION 1..8: note = peptide
DAPVTPDT

SEQ ID NO: 344 - protein, 8 aa, synthetic construct
  REGION 1..8: note = peptide
DAPVRPDS

SEQ ID NO: 345 - protein, 9 aa, synthetic construct
  REGION 1..9: note = peptide
YDRPVQPDR

SEQ ID NO: 346 - protein, 15 aa, synthetic construct
  REGION 1..15: note = peptide
GKTKEGVLYVGSKTK

SEQ ID NO: 347 - protein, 15 aa, synthetic construct
  REGION 1..15: note = peptide
KTKEGVLYVGSKTKE

SEQ ID NO: 348 - protein, 15 aa, synthetic construct
  REGION 1..15: note = peptide
EQVTNVGGAVVTGVT

SEQ ID NO: 349 - protein, 26 aa, synthetic construct
  REGION 1..26: note = peptide
VTGVTAVAQKTVEGAGNIAAATGFVK

SEQ ID NO: 350 - protein, 15 aa, synthetic construct
  REGION 1..15: note = peptide
MPVDPDNEAYEMPSE

SEQ ID NO: 351 - protein, 15 aa, synthetic construct
  REGION 1..15: note = peptide
DNEAYEMPSEEGYQD

SEQ ID NO: 352 - protein, 15 aa, synthetic construct
  REGION 1..15: note = peptide
EMPSEEGYQDYEPEA

SEQ ID NO: 353-354 - skipped (2 entries)

SEQ ID NO: 355 - protein, 8 aa, synthetic construct
  REGION 1..8: note = peptide
  VARIANT 1: note = 3-27 amino acids from the immunogenic peptide
  VARIANT 2: note = any amino acid
  VARIANT 3: note = any amino acid
XXXVVRAA

SEQ ID NO: 356 - protein, 20 aa, synthetic construct
  REGION 1..20: note = peptide
APDTRPAPGSTAPPAHGVTS

SEQ ID NO: 357 - protein, 19 aa, synthetic construct
  REGION 1..19: note = peptide
GVTSAPDTRPAPGSTPPAH

SEQ ID NO: 358-376 - skipped (19 entries)

SEQ ID NO: 377 - protein, 18 aa, synthetic construct
  REGION 1..18: note = peptide
KLLSLIKGVIVHRLEGVE

SEQ ID NO: 378-432 - skipped (55 entries)

SEQ ID NO: 433 - protein, 19 aa, synthetic construct
  REGION 1..19: note = peptide
ISITEIKGVIVHRIETILF

SEQ ID NO: 434 - skipped

SEQ ID NO: 435 - protein, 20 aa, synthetic construct
  REGION 1..20: note = peptide
NYSLDKIIVDYNLQSKITLP

SEQ ID NO: 436 - protein, 20 aa, synthetic construct
  REGION 1..20: note = peptide
LINSTKIYSYFPSVISKVNQ

SEQ ID NO: 437 - protein, 20 aa, synthetic construct
  REGION 1..20: note = peptide
LEYIPEITLPVIAALSIAES

SEQ ID NO: 438 - protein, 6 aa, synthetic construct
  REGION 1..6: note = peptide
DAEFRH

SEQ ID NO: 439-503 - skipped (65 entries)

SEQ ID NO: 504 - protein, 12 aa, synthetic construct
  REGION 1..12: note = peptide
AKFVAAWTLKAA

SEQ ID NO: 505-513 - skipped (9 entries)

SEQ ID NO: 514 - protein, 13 aa, synthetic construct
  REGION 1..13: note = peptide
  VARIANT 1: note = aliphatic amino acid residue selected from alanine, glycine, valine, isoleucine and leucine
  VARIANT 3: note = L cyclohexylalanine
  VARIANT 13: note = aliphatic amino acid residue selected from alanine, glycine, valine, isoleucine and leucine
XKXVAAWTLKAAX

SEQ ID NO: 515 - skipped

SEQ ID NO: 516 - protein, 13 aa, synthetic construct
  REGION 1..13: note = peptide
  VARIANT 1: note = aliphatic amino acid residue selected from alanine, glycine, valine, isoleucine and leucine
  VARIANT 13: note = aliphatic amino acid residue selected from alanine, glycine, valine, isoleucine and leucine
XKFVAAWTLKAAX

SEQ ID NO: 517 - protein, 9 aa, synthetic construct
  REGION 1..9: note = peptide
VLYVGSKTK

SEQ ID NO: 518 - protein, 15 aa, synthetic construct
  REGION 1..15: note = peptide
MPVDPDNEAYEMPSE

SEQ ID NO: 519 - protein, 15 aa, synthetic construct
  REGION 1..15: note = peptide
DNEAYEMPSEEGYQD

SEQ ID NO: 520 - protein, 15 aa, synthetic construct
  REGION 1..15: note = peptide
EMPSEEGYQDYEPEA

SEQ ID NO: 521 - protein, 15 aa, synthetic construct
  REGION 1..15: note = peptide
GKTKEGVLYVGSKTK

SEQ ID NO: 522 - protein, 15 aa, synthetic construct
  REGION 1..15: note = peptide
KTKEGVLYVGSKTKE

SEQ ID NO: 523 - protein, 15 aa, synthetic construct
  REGION 1..15: note = peptide
EQVTNVGGAVVTGVT

SEQ ID NO: 524 - protein, 26 aa, synthetic construct
  REGION 1..26: note = peptide
VTGVTAVAQKTVEGAGNIAAATGFVK

SEQ ID NO: 525 - protein, 12 aa, synthetic construct
  REGION 1..12: note = peptide
DPDNEAYEMPSE

SEQ ID NO: 526 - protein, 15 aa, synthetic construct
  REGION 1..15: note = peptide
DNEAYEMPSEEGYQD

SEQ ID NO: 527 - protein, 15 aa, synthetic construct
  REGION 1..15: note = peptide
EMPSEEGYQDYEPEA

SEQ ID NO: 528 - protein, 15 aa, synthetic construct
  REGION 1..15: note = peptide
EQVTNVGGAVVTGVT

SEQ ID NO: 529 - protein, 15 aa, synthetic construct
  REGION 1..15: note = peptide
GKTKEGVLYVGSKTK

SEQ ID NO: 530 - protein, 15 aa, synthetic construct
  REGION 1..15: note = peptide
KTKEGVLYVGSKTKE

SEQ ID NO: 531 - protein, 15 aa, synthetic construct
  REGION 1..15: note = peptide
EQVTNVGGAVVTGVT

SEQ ID NO: 532 - protein, 26 aa, synthetic construct
  REGION 1..26: note = peptide
VTGVTAVAQKTVEGAGNIAAATGFVK

SEQ ID NO: 533 - protein, 15 aa, synthetic construct
  REGION 1..15: note = peptide
MPVDPDNEAYEMPSE

SEQ ID NO: 534 - protein, 15 aa, synthetic construct
  REGION 1..15: note = peptide
DNEAYEMPSEEGYQD

SEQ ID NO: 535 - protein, 15 aa, synthetic construct
  REGION 1..15: note = peptide
EMPSEEGYQDYEPEA

SEQ ID NO: 536-537 - skipped (2 entries)

SEQ ID NO: 538 - protein, 9 aa, synthetic construct
  REGION 1..9: note = peptide
VDPDNEAYE

SEQ ID NO: 539 - protein, 26 aa, synthetic construct
  REGION 1..26: note = peptide
NEEGAPQEGILEDMPVDPDNEAYEMP

SEQ ID NO: 540 - protein, 7 aa, synthetic construct
  REGION 1..7: note = peptide
DNEAYEM

SEQ ID NO: 541 - protein, 7 aa, synthetic construct
DQPVLPD

SEQ ID NO: 542 - protein, 8 aa, synthetic construct
CDQPVLPD

SEQ ID NO: 543 - protein, 7 aa, synthetic construct
DMPVDPD

SEQ ID NO: 544 - protein, 8 aa, synthetic construct
CDMPVDPD

SEQ ID NO: 545 - protein, 8 aa, synthetic construct
MDVFMKGL

SEQ ID NO: 546 - protein, 9 aa, synthetic construct
MDVFMKGLC

SEQ ID NO: 547 - protein, 10 aa, synthetic construct
ATGFVKKDQL

SEQ ID NO: 548 - protein, 11 aa, synthetic construct
ATGFVKKDQLC

SEQ ID NO: 549 - protein, 9 aa, synthetic construct
LGKNEEGAP

SEQ ID NO: 550 - protein, 10 aa, synthetic construct
EGYQDYEPEA

SEQ ID NO: 551 - protein, 11 aa, synthetic construct
EGYQDYEPEAC

SEQ ID NO: 552 - protein, 8 aa, synthetic construct
DMPVDPDN

SEQ ID NO: 553 - protein, 9 aa, synthetic construct
CDMPVDPDN

SEQ ID NO: 554 - protein, 6 aa, synthetic construct
DMPVDP

SEQ ID NO: 555 - protein, 7 aa, synthetic construct
CDMPVDP

SEQ ID NO: 556 - protein, 5 aa, synthetic construct
DMPVD

SEQ ID NO: 557 - protein, 6 aa, synthetic construct
CDMPVD

SEQ ID NO: 558 - protein, 9 aa, synthetic construct
DMPVDPDNE

SEQ ID NO: 559 - protein, 10 aa, synthetic construct
DMPVDPDNEC

SEQ ID NO: 560 - protein, 10 aa, synthetic construct
DMPVDPDNEA

SEQ ID NO: 561 - protein, 11 aa, synthetic construct
DMPVDPDNEAC

SEQ ID NO: 562 - protein, 11 aa, synthetic construct
DMPVDPDNEAY

SEQ ID NO: 563 - protein, 12 aa, synthetic construct
DMPVDPDNEAYC

SEQ ID NO: 564 - protein, 12 aa, synthetic construct
DMPVDPDNEAYE

SEQ ID NO: 565 - protein, 13 aa, synthetic construct
DMPVDPDNEAYEC

SEQ ID NO: 566 - protein, 8 aa, synthetic construct
APQEGILE

SEQ ID NO: 567 - protein, 9 aa, synthetic construct
APQEGILEC